(19) United States
(12) Patent Application Publication
(10) Pub. No.: US 2026/0146229 A1
(43) Pub. Date: May 28, 2026

(54) PHOTORECEPTOR PRECURSOR CELL (PRP) COMPOSITIONS AND USES THEREOF

(71) Applicants: BlueRock Therapeutics LP, Cambridge, MA (US); Fujifilm Cellular Dynamics, Inc., Madison, WI (US)

(72) Inventors: Nicole Lund, Campbelville (CA); Young Mi Huang, Cambridge, MA (US); Ashwini Nirushika Mahendran, Pickering (CA); Angela Keightley, Scarborough (CA); Erin Dalzell, Newbury, MA (US); Lenore von Krusenstiern, Brookline, MA (US); Lucia Wille, Belmont, MA (US); Eric Donders, Etobicoke (CA); Stefanie Pohlod, Toronto (CA); Lucas Chase, Madison, WI (US); Kyle Wallace, Madison, WI (US); Benjamin Meline, Madison, WI (US); Andrew Dias, Madison, WI (US); Brandon Shelley, Madison, WI (US); Marisa Fenn, Madison, WI (US); Debjani Phillips, Madison, WI (US); Matthew Sternfeld, Madison, WI (US); Nathan Meyer, Madison, WI (US)

(21) Appl. No.: 19/235,357

(22) Filed: Jun. 11, 2025

Related U.S. Application Data
(60) Provisional application No. 63/726,133, filed on Nov. 27, 2024.
(60) Provisional application No. 63/808,222, filed on May 19, 2025.

(51) Int. Cl. C12N 5/0793; A61K 35/30; C12N 5/00

(52) U.S. Cl. CPC C12N 5/062; A61K 35/30; C12N 5/525

(57) ABSTRACT

The present invention relates to compositions comprising photoreceptor precursor cells (PRPs), as well as methods of generating the composition of PRPs and methods of using the composition of PRPs for regenerative cell therapy.

### FIELD

The present disclosure relates generally to the field of cellular-based therapy. More particularly, it concerns photoreceptor precursor (PRP) cells and methods of use thereof.

### BACKGROUND

Primary photoreceptor diseases are a subgroup of inherited retinal diseases that include retinitis pigmentosa and cone-rod dystrophies. These diseases affect the structure and function of the photoreceptor cells in the retina, leading to irreversible vision loss in both children and adults. Current treatments for primary photoreceptor disease are mostly supportive and primarily limited to the use of specialized low-vision aids such as eyeglasses, magnifiers, adaptation devices, and services for the blind. Thus, there remains a significant unmet medical need for patients with loss of photoreceptor cells.

### SUMMARY

The present disclosure addresses the above need by providing compositions of photoreceptor precursor cells, methods for generating photoreceptor precursor cells, and methods of use of photoreceptor precursor cells, including methods for treating an inherited retinal disease in a subject in need thereof by administering a therapeutically effective amount of photoreceptor precursor cells.

In an aspect, described herein is a composition of cells comprising photoreceptor precursor cells (PRPs), wherein the composition comprises: NR2E3+ cells and between at least about 20% to about 60% PPP4R4+ cells; wherein the PRPs comprise less than or equal to about 0.5 copies of VIM per copy of SDHA.

In an aspect, provided herein is a composition of cells comprising photoreceptor precursor cells (PRPs), wherein the composition comprises NR2E3+ cells and between at least about 20% to about 60% PPP4R4+ cells.

In some embodiments, at least about 20% to about 60% of the PRPs are PPP4R4+, wherein the cells are substantially NR2E3+, and wherein the cells comprise less than or equal to about 0.5 copies of VIM per copy of SDHA.

In some embodiments, the cells comprise at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

In some embodiments, a percentage NR2E3+, PPP4R4+, AIPL1+, RCVRN+, PAX6+, CHX10+, TYRP1+, and/or Ki67+ cells is determined by flow cytometry.

In some embodiments, copies of VIM per copy of SDHA is determined by droplet digital polymerase chain reaction (ddPCR).

In some embodiments, the cells have a percentage viability of at least about 50%.

In some embodiments, the percentage viability is determined by automated cell counting using acridine orange and propidium iodide.

In some embodiments, the cells are of human origin.

In some embodiments, the cells are derived from pluripotent stem cells (PSCs) or PSC-derived cells, optionally induced PSCs (iPSCs).

In an aspect, provided herein is a composition of cells consisting of photoreceptor precursor cells (PRPs) and, optionally, a pharmaceutically acceptable excipient thereof, wherein the cells consist of: NR2E3+ cells and between at least about 20% to about 60% PPP4R4+ cells; and wherein the PRPs comprise less than or equal to about 0.5 copies of VIM per copy of SDHA.

In some embodiments, the cells consist of at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

In some embodiments, a percentage NR2E3+, PPP4R4+, AIPL1+, RCVRN+, PAX6+, CHX10+, TYRP1+, and/or Ki67+ cells is determined by flow cytometry.

In some embodiments, copies of VIM per copy of SDHA is determined by droplet digital polymerase chain reaction (ddPCR).

In some embodiments the cells have a percentage viability of at least about 40%.

In some embodiments, the percentage viability is determined by automated cell counting using acridine orange and propidium iodide.

In some embodiments, the cells are of human origin.

In some embodiments, the cells are derived from pluripotent stem cells (PSCs) or PSC-derived cells, optionally induced PSCs (iPSCs).

In an aspect, provided herein is a composition of cells comprising PRPs, wherein the composition comprises cell aggregates, wherein the cell aggregates have a median diameter of about 30 μm to about 60 μm.

In some embodiments, the median diameter of the cell aggregates is determined by a particle size analyzer.

In some embodiments, each cell is NR2E3+ or PPP4R4+.

In some embodiments, the cells comprise between at least about 20% to about 60% PPP4R4+ cells and wherein the cells comprise less than or equal to about 0.5 copies of VIM per copy of SDHA.

In some embodiments, a percentage of NR2E3+ and/or PPP4R4+ cells is determined by flow cytometry.

In some embodiments, copies of VIM per copy of SDHA is determined by ddPCR.

In some embodiments, the cells are in a single cell form, an aggregate form, or a suspended form.

In some embodiments, the cells are in a single cell form or an aggregate form.

In some embodiments, the cells are in a suspended form.

In some embodiments, described herein is the composition comprising cells and a pharmaceutically acceptable carrier.

In some embodiments, the cells are suitable for grafting to a subretinal space of a subject's eye.

In an aspect, provided herein is a method of identifying a cell population for treatment of an eye disease or disorder, the method comprising: assaying for PPP4R4+ cells in a cell population, wherein the cell population comprises between at least about 20% to about 60% PPP4R4+ cells, and wherein and the cells comprise less than or equal to 0.5 copies of VIM per copy of SDHA.

In some embodiments, the method further comprises assaying for AIPL1+ and/or RCVRN+ cells in the cell population, wherein the cells are substantially NR2E3, at least about 90% of the cells in the cell population are AIPL1+; at least about 90% of the cells in the cell population are RCVRN+; no more than about 7% of the cells are PAX6+; no more than about 7% of the cells are CHX10+; no more than about 0.5% of the cells are TYRP1+; or no more than about 0.5% of the cells are Ki67+.

In some embodiments, a percentage of NR2E3+, PPP4R4+, AIPL1+, RCVRN+, PAX6+, CHX10+, TYRP1+, and/or Ki67+ cells is determined by flow cytometry.

In some embodiments, copies of VIM per copy of SDHA is determined by ddPCR.

In some embodiments, the cell population has a percentage viability of at least about 40%.

In some embodiments, the percentage viability is determined by an automated cell counting using acridine orange and propidium iodide.

In some embodiments, the cell population is of human origin.

In some embodiments, the cell population is derived from PSCs or PSC-derived cells, optionally iPSCs.

In some embodiments, the cell population is in a single cell form, an aggregate form, or a suspended form.

In some embodiments, the cell population is in a single cell form or an aggregate form.

In some embodiments, the cell population is in a suspended form.

In some embodiments, the cell population is suitable for grafting to a subretinal space of a subject's eye.

In some embodiments, the eye (e.g., ocular) disease or disorder is a retinal disorder.

In some embodiments, the retinal disorder is characterized by a loss of photoreceptors and/or a loss of photoreceptor cell function.

In some embodiments, the eye disease is a primary photoreceptor disease, including retinitis pigmentosa, cone-rod disease, rod-cone disease, cone dystrophy, and/or cone-rod dystrophy.

In some embodiments, the eye disease of disorder is an inherited retinal disease.

In some embodiments, the eye disease or disorder is Usher Syndrome.

In an aspect, provided herein is a method of identifying a suitable cell population for treatment of an eye disease, the method comprising a) obtaining a population of cells comprising PRPs; b) measuring a percentage of PPP4R4+ cells in a cell population; and c) measuring a copy number of VIM per copy SDHA; wherein the cell population is identified as a suitable cell population for treatment of an eye disease when the percentage of PPP4R4+ cells in the cell population is between about 20% and about 60%, and the copy number of VIM per copy of SDHA is less than or equal to about 0.5 copies.

In some embodiments, the method further comprises: d) measuring a percentage of AIPL1+ RCVRN+, PAX6+, CHX10+, TYRP1+; and/or Ki67+ cells in the cell population, wherein the cell population is identified as a suitable cell population for treatment of an eye disease when the percentage of AIPL1+ cells is least about 90%, the percentage of RCVRN+ cells is least about 90%, the percentage of PAX6+ cells is no more than about 7%, the percentage of CHX10+ cells is no more than about 7%, the percentage of TYRP1+ cells is no more than about 0.5%, and the percentage of Ki67+ cells is no more than about 0.5%.

In some embodiments, a percentage of PPP4R4+, AIPL1+, RCVRN+, PAX6+, CHX10+, TYRP1+, or Ki67+ cells is determined by flow cytometry.

In some embodiments, copies of VIM per copy of SDHA is determined by ddPCR.

In some embodiments, the cell population is of human origin.

In some embodiments, the cell population is derived from PSCs or PSC-derived cells, optionally iPSCs.

In some embodiments, the cell population is in a single cell form, an aggregate form, or a suspended form.

In some embodiments, the cell population is in a suspended form.

In some embodiments, the cell population is suitable for grafting to a subretinal space of a subject's eye.

In an aspect, provided herein is a composition comprising human PRPs for treatment of an eye disease or disorder, wherein the composition is in suspension form, and at least a portion of the composition is aggregated.

In some embodiments, the composition comprises 1.70×10⁶ total cells, 3.15×10⁶ total cells, 4.45×10⁶ total cells, or 5.90×10⁶ total cells in a 50 μL volume so that an administered dose comprises about 1×10⁶, about 2×10⁶, about 3×10⁶, or about 4×10⁶ total viable cells, respectively.

In some embodiments, the composition comprises 2.04×10⁶ total cells, 3.78×10⁶ total cells, 5.35×10⁶ total cells, or 7.10×10⁶ total cells in a 50 μL volume so that an administered dose comprises about 1×10⁶, about 2×10⁶, about 3×10⁶, or about 4×10⁶ total viable cells, respectively.

In some embodiments, a dose for administration comprises a) about 2.72×10⁷ total cells/mL to about 4.08×10⁷ total cells/mL are present in the composition; b) about 5.04×10⁷ total cells/mL to about 7.56×10⁷ total cells/mL are present in the composition; c) about 7.12×10⁷ total cells/mL to about 1.07×10⁸ total cells/mL are present in the composition; or d) about 9.44×10⁷ total cells/mL to about 1.42×10⁸ total cells/mL are present in the composition.

In some embodiments, the dose for administration further comprises balanced salt solution buffer supplemented with 0.2% human serum albumin (HSA).

In some embodiments, cells/mL is determined by automated cell counting using acridine orange and/or propidium iodide.

In an aspect, provided herein is a drug product comprising human PRPs comprising between at least about 20% to about 60% PPP4R4+ cells, wherein the drug product comprising the human PRPs are NR2E3+; and wherein the drug product comprising the human PRPs comprise less than or equal to about 0.5 copies of VIM per copy of SDHA.

In some embodiments, the drug product further comprises human PRPs comprising at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and/or less than or equal to about 0.5% Ki67+ cells.

In some embodiments, a percentage of NR2E3+, PPP4R4+, AIPL1+, RCVRN+, PAX6+, CHX10+, TYRP1+, and/or Ki67+ cells is determined by flow cytometry.

In some embodiments, copies of VIM per copy of SDHA is determined by ddPCR.

In some embodiments, the drug product comprising the human PRPs have a percentage viability of at least about 50%.

In some embodiments, the percentage viability is determined by automated cell counting using acridine orange and propidium iodide.

In some embodiments, the drug product comprising the human PRPs comprise cell aggregates wherein a median diameter of a cell aggregate is about 30 μm to about 60 μm.

In some embodiments, the median diameter of a cell aggregate is determined by a particle size analyzer.

In some embodiments, the drug product is resuspended in media.

In some embodiments, the drug product is resuspended in CryoSTOR® CS10 solution.

In some embodiments, the drug product is reformulated after thaw.

In some embodiments, the drug product comprises about 25×10⁶ cells.

In an aspect, provided herein is a composition comprising human PRPs for treatment of an eye disease or disorder, comprising a therapeutically effective amount of the composition, wherein the composition is in an aggregated form.

In some embodiments, the composition comprises 1.70×10⁶ total cells, 3.15×10⁶ total cells, 4.45×10⁶ total cells, or 5.90×10⁶ total cells in a 50 μL volume so that an administered dose comprises about 1×10⁶, about 2×10⁶, about 3×10⁶, or about 4×10⁶ total viable cells, respectively.

In some embodiments, the composition comprises 2.04×10⁶ total cells, 3.78×10⁶ total cells, 5.35×10⁶ total cells, or 7.10×10⁶ total cells in a 50 μL volume so that an administered dose comprises about 1×10⁶, about 2×10⁶, about 3×10⁶, or about 4×10⁶ total viable cells, respectively.

In some embodiments, a dose for administration comprises a) about 2.72×10⁷ total cells/mL to about 4.08×10⁷ total cells/mL are present in the composition; b) about 5.04×10⁷ total cells/mL to about 7.56×10⁷ total cells/mL are present in the composition; c) about 7.12×10⁷ total cells/mL to about 1.07×10⁸ total cells/mL are present in the composition; or d) about 9.44×10⁷ total cells/mL to about 1.42×10⁸ total cells/mL are present in the composition.

In some embodiments, the dose for administration is administered within 28 hours after reformulation from a drug product.

In some embodiments, cells/mL is determined by automated cell counting using acridine orange and/or propidium iodide.

In some embodiments, a drug product comprising the human PRPs comprises between at least about 20% to about 60% PPP4R4+ cells, wherein the PRPs are NR2E3+; and wherein the PRPs comprise less than or equal to about 0.5 copies of VIM per copy of SDHA.

In some embodiments, the composition comprises at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and/or less than or equal to about 0.5% Ki67+ cells.

In some embodiments, a percentage of NR2E3+, PPP4R4+, AIPL1+, RCVRN+, PAX6+, CHX10+, TYRP1+, and/or Ki67+ cells is determined by flow cytometry.

In some embodiments, copies of VIM per copy of SDHA is determined by ddPCR.

In some embodiments, the composition has a percentage viability of at least about 50%.

In some embodiments, the percentage viability is determined by automated cell counting using acridine orange and propidium iodide.

In some embodiments, the composition further comprises cell aggregates wherein a median diameter of a cell aggregate is about 30 μm to about 60 μm.

In some embodiments, the median diameter of a cell aggregate is determined by a particle size analyzer.

In an aspect, provided herein is a composition comprising human PRPs and a pharmaceutically acceptable carrier. In some embodiments, the pharmaceutically acceptable carrier is balanced salt solution buffer supplemented with about 0.2% human serum albumin (HSA).

In some embodiments, the human PRPs are stable at about 1-8° C. for about 4-31 hours.

In some embodiments, the human PRPs are stable at about 1-8° C. for about 4-7 hours.

In some embodiments, the human PRPs are stable at about 1-8° C. for about 24-27 hours.

In some embodiments, the human PRPs are stable at about 1-8° C. for about 28-31 hours.

In some embodiments, stability is assessed by one or more of percentage viability, viable cell concentration, and/or percentage aggregate biomass.

In some embodiments, the balanced salt solution buffer comprises about 0.3 mg/mL of magnesium chloride, about 0.48 mg/mL of calcium chloride, about 0.75 mg/mL of potassium chloride, about 1.7 mg/mL of sodium citrate, about 3.9 mg/mL of sodium acetate, and/or about 6.4 mg/mL of sodium chloride.

In an aspect, provided herein is a method for assessing whether a dose for administration comprising PRPs is compatible with a system for administration comprising a) exposing the PRPs to about 1-8° C.; b) agitating the PRPs; c) aspirating the PRPs into a cannula and syringe; d) optionally holding the PRPs in the syringe in a vertical orientation; e) injecting the PRPs from the cannula and syringe into a tube and/or cell culture plate; and f) measuring percentage aggregate biomass, percentage cell viability, and/or viable cell concentration of the PRPs in the tube and/or cell culture plate, wherein the dose for administration comprising PRPs is compatible with a system for administration if there is no substantial change in percentage aggregate biomass, percentage cell viability, and/or viable cell concentration of the dose for administration comprising PRPs compared to a composition comprising control PRPs.

### BRIEF DESCRIPTION OF THE OF THE DRAWINGS

The foregoing summary, as well as the following detailed description, will be better understood when read in conjunction with the appended figures. For the purpose of illustration, shown in the figures are embodiments. It should be understood, however, that the summary, detailed description, and figures are not limited to the precise arrangements, examples, and instrumentalities shown.

FIG. 1: depicts a particle size and percentage aggregate analysis acquired using a Multisizer 4e on a cell product comprising cell aggregates. The percentage volume of the solution is measured for each particle diameter (μm). Aggregates are characterized as having a diameter between 16.97 μm and 168.0 μm. The median diameter was 49.55 μm with 96.0% of cells present in aggregates.

FIG. 2: shows a representative image of aggregated cells acquired using a DMi1 inverted microscope at 20× magnification. Aggregated cells are shown as large clumps. Scale bar represents 100 μm.

FIG. 3: shows VIM gene expression level as measured by droplet digital polymerase chain reaction (ddPCR) with a reportable value, copies V/M/copy SDHA where target (VIM) expression (copies/μL) divided by reference gene (SDHA) expression (copies/μL). Non-clinical data generated using three non-representative drug product batches which have high VIM expression are shown in open circles. These data demonstrate negative outcomes with high VIM expression.

FIG. 4: shows a representative image of a cell product comprising cell aggregates acquired using the IncuCyte® Live-Cell Analysis System Spheroid Module software.

FIG. 5A-H: depicts a panel of graphs showing representative flow cytometric analyses of cell product comprising cell aggregates for 5A) AIPL1+ cells, 5B) RCVRN+ cells, 5C) PAX6+ cells, 5D) CHX10+ cells, 5E) TYRP1+ cells, 5F) Ki67+ cells, 5G) NR2E3+ cells, and 5H) PPP4R4+ cells.

### DETAILED DESCRIPTION

Provided herein are compositions of cells comprising photoreceptor precursor cells and methods of making or using the same. Such compositions may be useful in treating inherited (e.g., genetic) retinal diseases (e.g., a primary photoreceptor disease such as retinitis pigmentosa, Usher Syndrome, cone-rod disease, rod-cone disease, cone dystrophy, or cone-rod dystrophy, etc.). Such compositions may comprise an amount (e.g., a therapeutically effective amount) of photoreceptor precursor cells for administration to an eye of a subject in need thereof, such as a human. Advantageously, the compositions comprise substantially pure photoreceptor precursor cells that express PPP4R4 and/or NR2E3 (which indicates rod and/or cone photoreceptor development) and low expression of VIM. Such compositions are suitable for treating inherited retinal diseases such as a primary photoreceptor disease without the drawbacks associated with compositions of less purity.

### Definitions

Definitions of certain terms to be used herein are provided. Unless defined otherwise, all technical and scientific terms used herein have the same meaning as is commonly understood.

As used herein, the terms “cell”, “cell line”, and “cell culture” may be used interchangeably and all such designations include the progeny thereof.

The terms “cell dissociation” or “dissociation” refer to providing a cell composition containing cell aggregates or aggregated cells and separating the aggregated cells into singularized cells and/or reduced-size cell aggregates. Cell dissociation may be mechanical, physical, enzymatic, chemical, or any combination thereof. In an example, cell dissociation may be induced via incubation with TrypLE™ (Thermo Fisher Scientific, Waltham, MA; Catalog No.: A1217701). Cell dissociation may further involve agitation, vortexing, trituration, or mixing in order to break cell aggregates apart.

The terms “cells in aggregates,” “aggregated cells,” “aggregates,” “clumps,” or “cell aggregates” refer to cells associated with one another by means of intercellular adhesion or other mechanisms of association. In some embodiments, a cell suspension may comprise aggregated cells.

The term “cell population” or “cell composition” is used herein to refer to a group of cells. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The term “clinical significance” or “a clinically significant improvement,” used interchangeably herein, may be used to quantify treatment effectiveness, set clinical practice guidelines, and/or interpret trial results. The term relates to the practical importance of a treatment effect. For example, a clinically significant improvement may measure whether a treatment has a real genuine, palpable, noticeable effect on daily life of a patient or subject. A measure of clinical significance may differ based on the measurement, test, readout, protocol, trial, and/or patient outcome being assessed. Clinical significance may be measured by any methods know in the art for the corresponding measurements, tests, readouts, protocols, trials, and/or patient outcomes. Clinical significance may be an objective or subjective measure.

The phrase “composition of cells comprising photoreceptor precursor cells” refers to a cell population wherein the composition comprises at least about 90%, at least about 91%, at least about 92%, at least about 93%, at least about 94%, at least about 95%, at least about 96%, at least about 97%, at least about 98%, at least about 99%, or about 100% PRP cells.

The term “defined” or “fully defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, human plasma, or human serum. Generally, a defined medium comprises a basal media (e.g., Dulbecco's Modified Eagle's Medium (DMEM), F12, or Roswell Park Memorial Institute Medium (RPM1) 1640, containing amino acids, vitamins, inorganic salts, buffers, antioxidants, and energy sources) which is supplemented with recombinant albumin, chemically defined lipids, and/or recombinant insulin. An example of a fully defined medium is ESSENTIAL 8™ medium (Thermo Fisher Scientific, Waltham, MA; Cat. No. A1517001).

The term “differentiation” or “maturation” refers to the process by which an unspecialized cell becomes a more specialized type with changes in structural and/or functional properties. The mature cell typically has altered cellular structure and tissue-specific proteins.

As used herein, “essentially” or “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The terms “individual,” “host,” “subject,” “participant,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.). Preferably, the mammal is a human.

The term “induced pluripotent stem cells” or “iPSCs” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or at least four reprogramming factors to reprogram a somatic cell to a pluripotent stem cell.

The terms “inherited retinal disease” or “IRD” refer to a clinically and genetically diverse group of disorders characterized by outer retina degeneration and/or dysfunction. IRDs may involve impaired or complete loss of function of photoreceptor cells, leading to irreversible blindness. Some IRDs may be non-syndromic and affect only the eyes, whereas others may be considered syndromic, affecting the eyes as well as other organs.

An “isolated” cell has been substantially separated or purified from others cells in an organism or culture. Isolated cells can be, for example, at least about 99%, at least about 98% pure, at least about 95% pure, or at least about 90% pure.

The term “neural retinal progenitors” or “NRPs” refers to cells which are restricted in their differentiation potential to neural retina cell types.

The term “outer nuclear layer” or “ONL” refers to the light-detecting portion of the eye in a vertebrate. The ONL may include rod cell nuclei, cone cell nuclei, both rod and cone cell nuclei, or neither rod nor cone cell nuclei.

The term “outer retinal layer” refers to a layer of the retina that includes the ONL, as well as other layers such as the outer plexiform layer (OPL), the Henle fiber layer, the myoid and ellipsoid zone (MEZ), and the photoreceptor outer segments (OS).

The terms “photoreceptor precursor cells,” “photoreceptor progenitor cells,” “PRP cells,” or “PRPs” refer to cells differentiated from embryonic stem cells or induced pluripotent stem cells which can differentiate into photoreceptor cells that expresses the cell marker rhodopsin or any of the three cone opsins, and optionally express the rod or cone cyclic guanosine monophosphate (cGMP). PRP cells and/or compositions comprising PRPS do not express or have substantially no expression of CRALBP, BEST1, IGFBP5, COL8A1, ELN, UPK3B, TPM2, PTGDS, SERPINE3, TIMP3, MITF, and/or PMEL17. Additionally, PRP cells have low or substantially no expression of PAX6, CHX10, TYRP1, Ki67, and/or ONECUT1. The photoreceptors may be rod and/or cone photoreceptors. The plasma membrane of a photoreceptor cell contains two compartments: outer segments and inner segments. The term “outer segments” refers to a specialized compartment of rod and/or cone photoreceptor cells where phototransduction (e.g., capture of light and its conversion into electrical signals) takes place. The term “inner segments” refers to the compartment of the rod and/or cone cells where metabolism and membrane potential regulation occurs.

The “Photoreceptor Precursor Induction Medium” or “FDSC” refers to a growth medium which comprises a TGFβ inhibitor, a WNT inhibitor, and/or a γ-secretase inhibitor. The FDSC may comprise basic FGF and ascorbic acid.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell may be an embryonic stem cell derived from the inner cell mass of a blastocyst. In other embodiments, the pluripotent stem cell may be an induced pluripotent stem cell derived by reprogramming somatic cells.

“Pre-confluent” refers to a cell culture in which the proportion of the culture surface which is covered by cells is about 60% to about 80%. Usually, pre-confluent refers to a culture in which about 70% of the culture surface is covered by cells.

The terms “primary photoreceptor disease” or “PPD” refer to a subclass of inherited retinal diseases that include degenerative and non-degenerative diseases. Examples of degenerative PPDs may include retinitis pigmentosa, Usher Syndrome, cone-rod disease, rod-cone disease, cone dystrophy, and cone-rod dystrophy. Examples of non-degenerative PRDs may include achromatopsia. PPDs may be non-syndromic or syndromic and may be familial or sporadic.

The term “purified” does not require absolute purity; rather, it is intended as a relative term. Thus, a purified population of cells is greater than about 90% 91%, 92%, 93%, 94%, 95%, 96%, 97%, 98%, 99% or 100% pure, or, preferably, essentially free of other cell types.

The “Retinal Differentiation Medium,” “RD medium,” or “RD” is defined herein as a medium that comprises a WNT inhibitor, a TGFβ inhibitor, and a MEK inhibitor and differentiates anterior neuroectoderm cells. The RD medium may (e.g., RD1) or may not (e.g., RD1) comprise a BMP inhibitor and may comprise IGF-1 and ascorbic acid.

“Retinal Induction Medium (RIM)” refers herein to a growth media that comprises a WNT pathway inhibitor and a BMP pathway inhibitor and can result in the differentiation of PSCs to retinal lineage cells. The RIM may also comprise a TGFβ pathway inhibitor and may comprise IGF-1 and ascorbic acid.

The “Retinal Maturation Medium,” or “RM medium,” or “RM” is defined as a growth medium for the culture of retinal cells comprising nicotinamide and ascorbic acid. The RM is preferably free of Activin A. The RM may (e.g., RM1) or may not (e.g., RM2) comprise a γ-secretase inhibitor, such as DAPT, or a TGFβ inhibitor, such as SB431542 and may comprise IGF-1 and ascorbic acid.

The term “retinal pigment epithelium” or “RPE” refers to a layer of pigmented cells between the choroid, a layer filled with blood vessels, and the neural retina.

The term “retinal progenitor cells”, also called “retinal precursor cells” or “RPCs”, encompass cells which are competent for generating all cell types of the retina, including neural retina cells, such as rods, cones, photoreceptor precursor cells, as well as cells which can differentiate and/or mature into retinal pigment epithelium (RPE) cells.

As used herein, the term “stable” refer to cells which are able to withstand one or more conditions (e.g., elevated temperature, etc.) for a period of time. For example, stable cells have a less than 95%, 90%, 80%, 70%, 60%, 50%, 40%, 30%, 20%, 15%, 10%, 5%, or 1% decrease in viability over a period of time (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours or more) at about 1° C. to about 8° C. (e.g., about 1° C., about 2° C., about 3° C., about 4° C., about 5° C., about 6° C., about 7° C., or about 8° C.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

A “therapeutically effective amount” used herein refers to the amount of a compound or cells (e.g., PRPs) that, when administered to a subject for treatment of a disease or condition, is sufficient to affect such treatment. As used herein, the term “effective amount” means that dose of a composition comprising PRP cells that will elicit the biological or medical response of a tissue, system, animal, or human that is being sought, for instance, by a researcher or clinician. Furthermore, the term “therapeutically effective dose” means any dose that, as compared to a corresponding subject who has not received such dose, results in improved treatment, healing, or amelioration of a disease, disorder, or side effect, or a decrease in the rate of advancement of a disease or disorder. The term also includes within its scope doses effective to enhance normal physiological function. Therapeutically effective amounts and treatment regimens are generally determined empirically and may be dependent on factors, such as the age, weight, and health status of the subject and disease or disorder to be treated. Such factors are within the purview of the attending physician.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, whether or not the treatment leads to amelioration of the disease, comprises “treatment” or “therapy” as used herein. Moreover, as long as the compositions of the disclosure either alone or in combination with another therapeutic agent alleviate or ameliorate at least one symptom of a disorder being treated as compared to that symptom in the absence of use of a composition comprising PRP cells, the result should be considered an effective treatment of the underlying disorder regardless of whether all the symptoms of the disorder are alleviated or not.

As used herein, “undifferentiated” refers to cells that display characteristic markers and morphological characteristics of undifferentiated cells that clearly distinguish them from terminally differentiated cells of embryo or adult origin.

For a medium, extracellular matrix, or culture system used with human cells, the term “Xeno-Free (XF)” refers to a condition in which the materials used are not of non-human animal origin.

### Photoreceptor Precursor Cell (PRP) Compositions

In an aspect, at least about 80% of the composition comprising the population of PRP cells expresses RCVRN (e.g., about 80%, about 81%, about 82%, about 83%, about 84%, about 85%, about 86%, about 87%, about 88%, about 89%, about 90%, about 91%, about 92%, about 93%, about 94%, about 95% about 96%, about 97%, about 98% about 99%, or about 100% of the composition comprising the population of PRP cells expresses RCVRN). In particular embodiments, the disclosure provides a composition comprising a population of PRP cells. In some embodiments, about 80% to about 100% (e.g., about 80% to about 100%, about 80% to about 90%, about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%) of the composition comprising the population of PRP cells expresses Recoverin (RCVRN). In some embodiments, the composition comprising the population of PRP cells comprises at least about 90% of RCVRN expressing cells.

In an aspect, the disclosure provides a composition comprising a population of PRP cells. In particular embodiments, at least about 80% of the composition comprising the population of PRP cells expresses AIPL1 (e.g., about 80%, about 81%, about 82%, about 83%, about 84%, about 85%, about 86%, about 87%, about 8%, about 89%, about 90%, about 91%, about 92%, about 93%, about 94%, about 95%, about 96%, about 97%, about 98%, about 99%, or about 100% of the composition comprising the population of PRP cells expresses AIPL1). In some embodiments, about 90% to about 100% (e.g., about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%) of the composition comprising the population of PRP cells expresses aryl-hydrocarbon interacting protein-like 1 (AIPL1). In some embodiments, the composition comprising the population of PRP cells comprises at least about 90% of AIPL1-expressing cells.

In some embodiments, the composition comprising the population of PRP cells comprises at least about 20% PPP4R4-expressing cells (e.g., at least about 20%, about 21%, at least 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% PPP4R4-expressing cells). In some embodiments, the composition comprising the population of PRP cells comprises at most about 60% (e.g., about 20%, about 21%, at least 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% PPP4R4-expressing cells). In some embodiments, the composition comprising the population of PRP cells comprises between about 20% and about 60% (e.g., about 20% to about 60%, about 20% to about 50%, about 20% to about 40%, about 20% to about 30%, about 30% to about 60%, about 30% to about 50%, about 30% to about 40%, about 40% to about 60%, about 40% to about 50%, or about 50% to about 60%) PPP4R4 expressing cells. In some embodiments, PPP4R4 expressing cells are cone-committed PRPs. In some embodiments, the composition comprising the population of PRP cells comprises at least about 20% PPP4R4-expressing cells. In some embodiments, the composition comprising the population of PRP cells comprises at most about 60% PPP4R4-expressing cells. In some embodiments, the composition comprising the population of PRP cells comprises at least about 20% of PPP4R4-expressing cells and at most about 60% PPP4R4-expressing cells.

In some embodiments, the composition comprising the population of PRP cells comprises at least about 10% to at most about 100% NR2E3-expressing cells (e.g., at least about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% NR2E3-expressing cells). In some embodiments, the composition comprising the population of PRP cells comprises between about 10% and about 100% (e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%) NR2E3-expressing cells. In some embodiments, NR2E3 expressing cells are rod-committed PRPs. In some embodiments, the composition comprising the population of PRP cells comprises at least about 25% to at most about 65% NR2E3-expressing cells. In some embodiments, the composition comprising the population of PRP cells comprises at least about 40% to at most about 80% NR2E3-expressing cells.

In some embodiments, a composition comprising the population of cells comprising 90% PRP cells comprises at least about 90% AIPL1-expressing cells and at least about 90% RCVRN-expressing cells. In further embodiments, of those 90% PRPs expressing AIPL1 and RCVRN, a first portion will express PPP4R4 and a second portion will express NR2E3. In further embodiments, of those 90% PRPs expressing AIPL1 and RCVRN, the first portion will express PPP4R4 but will not express NR2E3. In further embodiments, of those 90% PRPs expressing AIPL1 and RCVRN, the second portion will express NR2E3 but will not express PPP4R4. In further embodiments, of those 90% PRPs expressing AIPL1 and RCVRN, at least about 20% of the cells will express PPP4R4, at least about 80% of the cells will express NR2E3. In further embodiments, of those 90% PRPs expressing AIPL1 and RCVRN, at least about 30% of the cells will express PPP4R4, at least about 70% of the cells will express NR2E3. In further embodiments, of those 90% PRPs expressing AIPL1 and RCVRN, at least about 40% of the cells will express PPP4R4, at least about 60% of the cells will express NR2E3. In further embodiments, of those 90% PRPs expressing AIPL1 and RCVRN, at least about 50% of the cells will express PPP4R4, at least about 50% of the cells will express NR2E3. In further embodiments, of those 90% PRPs expressing AIPL1 and RCVRN, at least about 60% of the cells will express PPP4R4, at least about 40% of the cells will express NR2E3.

In some embodiments, the composition comprising the population of PRP cells comprises about 0.0 copies to about 0.5 copies VIM per copy of SDHA as measured by ddPCR, (e.g., about 0.0, about 0.05, about 0.10, about 0.15, about 0.20, about 0.25, about 0.30, about 0.35, about 0.40, about 0.45, or about 0.50 copies VIM per copy SDHA as measured by ddPCR). In some embodiments, the composition comprising the population of PRP cells comprises VIM+ cells. In some embodiments, the composition comprising the population of PRP cells comprises between about 0.0 copies and about 0.50 copies (e.g., about 0.0 copies to about 0.1, about 0.0 copies to about 0.2, about 0.0 copies to about 0.3, about 0.0 copies to about 0.4, about 0.0 copies to about 0.5, about 0.1 copies to about 0.2, about 0.1 copies to about 0.3, about 0.1 copies to about 0.4, about 0.1 copies to about 0.5, about 0.2 copies to about 0.3, about 0.2 copies to about 0.4, about 0.2 copies to about 0.5, about 0.3 copies to about 0.4, about 0.3 copies to about 0.5, or about 0.4 copies to about 0.5) VIM per copy of a reference gene, e.g., SDHA, as measured by ddPCR. In some embodiments, the composition comprising the population of PRP cells comprises at most 0.5 copies VIM per copy SDHA as measured by ddPCR. In some embodiments, ddPCR is performed using Bio-Rad's QX200™ ddPCR System. In some embodiments, copies of VIM is determined relative to reference gene SDHA.

In some embodiments, the composition comprising the population of PRP cells comprises at most 7% PAX6-expressing cells (e.g., about 7.0%, about 6.75%, about 6.50%, about 6.25%, about 6.0%, about 5.75%, about 5.50%, about 5.25%, about 5.0%, about 4.75%, about 4.50%, about 4.25%, about 4.0%, about 3.75%, about 3.50%, about 3.25%, about 3.0%, about 2.75%, about 2.50%, about 2.25%, about 2.0%, about 1.75%, about 1.50%, about 1.25%, about 1.0%, about 0.75%, about 0.50%, about 0.25%, or about 0.0% PAX6-expressing cells). In some embodiments, the composition comprising the population of PRP cells comprises between about 0% and about 7% (e.g., about 0% to about 7%, about 0% to about 6%, about 0% to about 5%, about 0% to about 4%, about 0% to about 3%, about 0% to about 2%, about 0% to about 1%, about 1% to about 7%, about 1% to about 6%, about 1% to about 5%, about 1% to about 4%, about 1% to about 3%, about 1% to about 2%, about 2% to about 7%, about 2% to about 6%, about 2% to about 5%, about 2% to about 4%, about 2% to about 3%, about 3% to about 7%, about 3% to about 6%, about 3% to about 5%, about 3% to about 4%, about 4% to about 7%, about 4% to about 6%, about 4% to about 5%, about 5% to about 7%, about 5% to about 6%, or about 6% to about 7%) PAX6-expressing cells. In some embodiments, the composition comprising the population of PRP cells comprises at most about 7% PAX6-expressing cells.

In some embodiments, the composition comprising the population of PRP cells comprises at most 5% CHX10-expressing cells (e.g., about 7.0%, about 6.75%, about 6.50%, about 6.25%, about 6.0%, about 5.75%, about 5.50%, about 5.25%, about 5.0%, about 4.75%, about 4.50%, about 4.25%, about 4.0%, about 3.75%, about 3.50%, about 3.25%, about 3.0%, about 2.75%, about 2.50%, about 2.25%, about 2.0%, about 1.75%, about 1.50%, about 1.25%, about 1.0%, about 0.75%, about 0.50%, about 0.25%, or about 0.0% CHX10-expressing cells). In some embodiments, the composition comprising the population of PRP cells comprises between about 0% and about 7% (e.g., about 0% to about 7%, about 0% to about 6%, about 0% to about 5%, about 0% to about 4%, about 0% to about 3%, about 0% to about 2%, about 0% to about 1%, about 1% to about 7%, about 1% to about 6%, about 1% to about 5%, about 1% to about 4%, about 1% to about 3%, about 1% to about 2%, about 2% to about 7%, about 2% to about 6%, about 2% to about 5%, about 2% to about 4%, about 2% to about 3%, about 3% to about 7%, about 3% to about 6%, about 3% to about 5%, about 3% to about 4%, about 4% to about 7%, about 4% to about 6%, about 4% to about 5%, about 5% to about 7%, about 5% to about 6%, or about 6% to about 7%) CHX10-expressing cells. In some embodiments, the composition comprising the population of PRP cells comprises at most about 7% CHX10-expressing cells.

In some embodiments, the composition comprising the population of PRP cells comprises at most about 0.5% Ki67-expressing cells (e.g., about 0.00%, about 0.05%, about 0.10%, about 0.15%, about 0.20%, about 0.25%, about 0.30%, about 0.35%, about 0.40%, about 0.45%, or about 0.50% Ki67-expressing cells). In some embodiments, the composition comprising the population of PRP cells comprises between about 0% and about 0.5% (e.g., about 0.0% to about 0.1%, about 0.0% to about 0.2%, about 0.0% to about 0.3%, about 0.0% to about 0.4%, about 0.0% to about 0.5%, about 0.1% to about 0.2%, about 0.1% to about 0.3%, about 0.1% to about 0.4%, about 0.1% to about 0.5%, about 0.2% to about 0.3%, about 0.2% to about 0.4%, about 0.2% to about 0.5%, about 0.3% to about 0.4%, about 0.3% to about 0.5%, or about 0.4% to about 0.5%) Ki67-expressing cells. In some embodiments, the composition comprising the population of PRP cells comprises at most 0.5% Ki67-expressing cells.

In some embodiments, the composition comprising the population of PRP cells comprises at most 0.5% TYRP1-expressing cells (e.g., about 0.00%, about 0.05%, about 0.10%, about 0.15%, about 0.20%, about 0.25%, about 0.30%, about 0.35%, about 0.40%, about 0.45%, or about 0.50% TYRP1-expressing cells. In some embodiments, the composition comprising the population of PRP cells comprises between about 0% and about 0.5% (e.g., about 0.0% to about 0.1%, about 0.0% to about 0.2%, about 0.0% to about 0.3%, about 0.0% to about 0.4%, about 0.0% to about 0.5%, about 0.1% to about 0.2%, about 0.1% to about 0.3%, about 0.1% to about 0.4%, about 0.1% to about 0.5%, about 0.2% to about 0.3%, about 0.2% to about 0.4%, about 0.2% to about 0.5%, about 0.3% to about 0.4%, about 0.3% to about 0.5%, or about 0.4% to about 0.5%) TYRP1− expressing cells. In some embodiments, the composition comprising the population of PRP cells comprises at most 0.5% TYRP1-expressing cells.

In some embodiments, the composition comprising the population of PRP cells comprises NR2E3+ cells (e.g., at an amount or percentage described herein), between at least about 20% and at most about 60% PPP4R4+ cells, at most 0.5 copies VIM per copy SDHA, at least about 90% AIPL1+ cells, at least about 90% RCVRN+ cells, at most about 7% PAX6+ cells, at most about 7% CHX10+ cells, at most about 0.5% TYRP1+ cells, and/or at most about 0.5% Ki67+ cells. In some embodiments, the composition comprising the population of PRP cells comprises between at least about 20% and at most about 60% PPP4R4+ cells and at most 0.5 copies VIM per copy SDHA. In some embodiments, the composition comprising the population of PRP cells comprises NR2E3+ cells and between at least about 20% and at most about 60% PPP4R4+ cells. In some embodiments, the composition comprising the population of PRP cells comprises NR2E3+ cells, between at least about 20% and at most about 60% PPP4R4+ cells, and at most 0.5 copies VIM per copy SDHA. In some embodiments, the composition comprising the population of PRP cells comprises at least about 90% AIPL1+ cells and at least about 90% RCVRN+ cells. In some embodiments, the composition comprising the population of PRP cells comprises NR2E3+ cells, between at least about 20% and at most about 60% PPP4R4+ cells, at most 0.5 copies VIM per copy SDHA, at least about 90% AIPL1+ cells, and at least about 90% RCVRN+ cells. In some embodiments, the composition comprising the population of PRP cells comprises at most about 7% PAX6+ cells, at most about 7% CHX10+ cells, at most about 0.5% TYRP1+ cells, and/or at most about 0.5% Ki67+ cells. In some embodiments, the composition comprising the population of PRP cells comprises NR2E3+ cells and between at least about 20% to about 60% PPP4R4+ cells. In some embodiments, the composition comprising the population of PRP cells comprises between about 40% and about 80% NR2E3+ cells, between at least about 20% and at most about 60% PPP4R4+ cells, at most 0.5 copies VIM per copy SDHA, at least about 90% AIPL1+ cells, at least about 90% RCVRN+ cells, at most about 7% PAX6+ cells, at most about 7% CHX10+ cells, at most about 0.5% TYRP1+ cells, and/or at most about 0.5% Ki67+ cells.

In certain aspects, the composition comprising the population of PRP cells does not express or have substantially no expression of CRALBP, BEST1, IGFBP5, COL8A1, ELN, UPK3B, TPM2, PTGDS, SERPINE3, TIMP3, MITF, and/or PMEL17. In some embodiments, the composition comprising the population of PRP cells has low or substantially no expression of PAX6, CHX10 (also referred to as VSX2), Ki67, TYRP1, and/or ONECUT1. In particular embodiments, at most about 15%, (e.g., about 0% to about 15%, about 0% to about 14%, about 0% to about 13%, about 0% to about 12%, about 0% to about 11%, about 0% to about 10%, about 0% to about 9%, about 0% to about 8%, about 0% to about 7%, about 0% to about 6%, about 0% to about 5%, about 0% to about 4%, about 0% to about 3%, about 0% to about 2%, about 0% to about 1%, about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 2% to about 4%, about 2% to about 3%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6%, about 3% to about 5%, about 3% to about 4%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 4% to about 5%, about 5% to about 15%, about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, or about 14% to about 15%) of the composition comprising the population of PRP cells express PAX6. In some embodiments, the composition comprising the population of PRP cells comprises at most about 10% or about 5% PAX6-positive cells. In certain embodiments, the composition comprising the population of PRP cells comprises at most about 7% PAX6-positive cells, at most about 0.05% Ki67-positive cells, at most about 30%, about 31%, about 32%, about 33%, about 34%, about 35%, about 36%, about 37%, about 38%, about 39%, about 40%, about 45%, about 50%, about 55%, about 60%, about 70%, about 80%, about 90%, about 95%, or about 100% CHX10-positive cells, and/or at most about 2% ONECUT1-positive cells. In some aspects, the composition comprising the population of PRP cells comprises at most about 7% PAX6-positive cells, at most about 0.5% Ki67-positive cells, at most about 7% CHX10-positive cells, and/or at most about 1% ON ECUT1-positive cells. In certain aspects, the composition comprising the population of PRP cells comprises at most about 7% PAX6-positive cells, at most about 0.5% Ki67-positive cells, at most about 7% CHX10-positive cells, and at most about 1% ONECUT1-positive cells.

In some embodiments, at least about 90% of the composition comprising the population of PRPs expresses TUBB3 (e.g., 90%, about 91%, about 92%, about 93%, about 94%, about 95%, about 96%, about 97%, about 98%, about 99%, or about 100% of the composition comprising the population of PRP cells expresses TUBB3). In some embodiments, at least about 90% to about 100 (e.g., about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%) of the composition comprising the population of PRP cells expresses Class Ill b-tubulin (TUBB3). In some embodiments, the composition comprising the population of PRP cells expresses one or more markers selected from the group consisting of: AIPL1, NR2E3, PPP4R4, OTX2, CRX, BLIMP 1, NEUROD1, RCVRN, TUBB3 and CD171/L1CAM.

In particular embodiments, the composition comprising the population of PRP cells comprises at least about 70% cell aggregate biomass (e.g., about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% cell aggregate biomass). In some embodiments, the composition comprising the population of PRP cells comprises between about 70% and about 100% (e.g., about 70% to about 100%, about 70% to about 90%, about 70% to about 80%, about 80% to about 100%, about 80% to about 90%, or about 90% to about 100%) cell aggregate biomass. In some embodiments, the composition comprising the population of PRP cells comprises at least about 70% cell aggregate biomass.

In some embodiments, a composition of cells comprising PRPs as disclosed herein, comprises, or consists of, or consists essentially of, one or more, two or more, three or more, four or more, five or more, six or more, seven or more, eight or more, nine or more, ten or more, eleven or more, or twelve of the characteristics provided in Table 1.

TABLE 1
Specifications for Drug Products Comprising a Composition
of Cells Comprising PRPs as Disclosed Herein

| Specification | Characteristic |
|---|---|
| AIPL1+ | At least 90% |
| RCVRN+ | At least 90% |
| PPP4R4+ | At least 20% and at most 60% |
| NR2E3+ | Detectable |
| PAX6+ | At most 7% |
| CHX10+ | At most 7% |
| TYRP1+ | At most 0.5% |
| Ki67+ | At most 0.5% |
| Copies of VIM per copy of SDHA | At most 0.5 copies/copy |
| Aggregate biomass | At least 70% |
| Percentage Viability | At least 50% |
| LNCPRESS2 and AC009446.1 | Not detectable |

In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, and wherein the composition is mostly in aggregate form, which is in suspension or pellet form.

In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, at least 20% and at most 60% PPP4R4+ cells, and wherein the composition is mostly in aggregate form, which is in suspension or pellet form. In some embodiments, a composition of cells comprising PRPs as disclosed herein, comprises, or consists of, or consists essentially of at least 90% RCVRN+ cells, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, a composition of cells comprising PRPs as disclosed herein, comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least 20% and at most 60% PPP4R4+ cells, and wherein the composition is mostly in aggregate form, which is in suspension or pellet form.

In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, at most 7% CHX10+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most 7% PAX6+ cells, and wherein the composition is mostly in aggregate form, which is in suspension or pellet form. In some embodiments, a composition of cells comprising PRPs as disclosed herein, comprises, or consists of, or consists essentially of NR2E3+ cells, and wherein the composition is mostly in aggregate form, which is in suspension or pellet form.

In some embodiments, a composition of cells comprising PRPs as disclosed herein, comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at most 7% PAX6+ cells, at most 7% CHX10+ cells, and wherein the composition is mostly in aggregate form, which is in suspension or pellet form. In some embodiments, a composition of cells comprising PRPs as disclosed herein, comprises, or consists of, or consists essentially of at most 7% PAX6+ cells, and wherein the composition is mostly in aggregate form, which is in suspension or pellet form.

In some embodiments, a composition of cells comprising PRPs as disclosed herein, comprises, or consists of, or consists essentially of at most 7% CHX10+ cells, at most 0.5% TYRP1+ cells, at most 0.5% Ki67+ cells, at most 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most 7% CHX10+ cells, at most 0.5% TYRP1+ cells, at most 0.5% Ki67+ cells, at most 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most 7% CHX10+ cells, at most 0.5% TYRP1+ cells, at most 0.5% Ki67+ cells, at most 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most 7% CHX10+ cells, at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most 7% CHX10+ cells, at most 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at most 7% CHX10+ cells, and wherein the composition is mostly in aggregate form, which is in suspension or pellet form.

In some embodiments, a composition of cells comprising PRPs as disclosed herein, comprises, or consists of, or consists essentially of at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most 0.5% TYRP1+ cells, at most 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most 0.5% TYRP1+ cells, and wherein the composition is mostly in aggregate form, which is in suspension or pellet form.

In some embodiments, a composition of cells comprising PRPs as disclosed herein, comprises, or consists of, or consists essentially of at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at most 0.5% Ki67+ cells, at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at most 0.5% Ki67+ cells, at most 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most 0.5% Ki67+ cells, and wherein the composition is mostly in aggregate form, which is in suspension or pellet form.

In some embodiments, a composition of cells comprising PRPs as disclosed herein, comprises, or consists of, or consists essentially of at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most 0.5 copies of VIM per copy of SDHA, at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at most 0.5 copies of VIM per copy of SDHA, at least 70% aggregate biomass, and wherein the composition is mostly in aggregate form, which is in suspension or pellet form. In some embodiments, a composition of cells comprising PRPs as disclosed herein, comprises, or consists of, or consists essentially of at most 0.5 copies of VIM per copy of SDHA, and wherein the composition is mostly in aggregate form, which is in suspension or pellet form.

In some embodiments, a composition of cells comprising PRPs as disclosed herein, comprises, or consists of, or consists essentially of at least 70% aggregate biomass, a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least 70% aggregate biomass, a percentage viability of at least 50%, and wherein the composition is mostly in aggregate form, which is in suspension or pellet form. In some embodiments, a composition of cells comprising PRPs as disclosed herein, comprises, or consists of, or consists essentially of at least 70% aggregate biomass, and wherein the composition is mostly in aggregate form, which is in suspension or pellet form.

In some embodiments, a composition of cells comprising PRPs as disclosed herein, comprises, or consists of, or consists essentially of a percentage viability of at least 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of a percentage viability of at least 50%, and wherein the composition is mostly in aggregate form, which is in suspension or pellet form.

In some embodiments, a composition of cells comprising PRPs as disclosed herein, comprises, or consists of, or consists essentially of no detectable expression of LNCPRESS2 and/or AC009446.1, and wherein the composition is mostly in aggregate form, which is in suspension or pellet form.

In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, and wherein the composition is mostly in aggregate form, which is in suspension or pellet form.

In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, at least about 20% and at most about 60% PPP4R4+ cells, and wherein the composition is mostly in aggregate form, which is in suspension or pellet form. In some embodiments, a composition of cells comprising PRPs as disclosed herein, comprises, or consists of, or consists essentially of at least about 90% RCVRN+ cells, and wherein the composition is mostly in aggregate form, which is in suspension or pellet form.

In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, a composition of cells comprising PRPs as disclosed herein, comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, NR2E3+ cells, and wherein the composition is mostly in aggregate form, which is in suspension or pellet form. In some embodiments, a composition of cells comprising PRPs as disclosed herein, comprises, or consists of, or consists essentially of at least about 20% and at most about 60% PPP4R4+ cells, and wherein the composition is mostly in aggregate form, which is in suspension or pellet form.

In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, at most about 7% CHX10+ cells, and wherein the composition is mostly in aggregate form, which is in suspension or pellet form. In some embodiments, a composition of cells comprising PRPs as disclosed herein, comprises, or consists of, or consists essentially of NR2E3+ cells, at most about 7% PAX6+ cells, and wherein the composition is mostly in aggregate form, which is in suspension or pellet form. In some embodiments, a composition of cells comprising PRPs as disclosed herein, comprises, or consists of, or consists essentially of NR2E3+ cells, and wherein the composition is mostly in aggregate form, which is in suspension or pellet form.

In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, at most about 7% CHX10+ cells, and wherein the composition is mostly in aggregate form, which is in suspension or pellet form. In some embodiments, a composition of cells comprising PRPs as disclosed herein, comprises, or consists of, or consists essentially of at most about 7% PAX6+ cells, and wherein the composition is mostly in aggregate form, which is in suspension or pellet form.

In some embodiments, a composition of cells comprising PRPs as disclosed herein, comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, at most about 0.5% TYRP1+ cells, and wherein the composition is mostly in aggregate form, which is in suspension or pellet form. In some embodiments, a composition of cells comprising PRPs as disclosed herein, comprises, or consists of, or consists essentially of at most about 7% CHX10+ cells, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form.

In some embodiments, a composition of cells comprising PRPs as disclosed herein, comprises, or consists of, or consists essentially of at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, at most about 0.5% Ki67+ cells, and wherein the composition is mostly in aggregate form, which is in suspension or pellet form. In some embodiments, a composition of cells comprising PRPs as disclosed herein, comprises, or consists of, or consists essentially of at most about 0.5% TYRP1+ cells, and wherein the composition is mostly in aggregate form, which is in suspension or pellet form.

In some embodiments, a composition of cells comprising PRPs as disclosed herein, comprises, or consists of, or consists essentially of at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most about 0.5% Ki67+ cells, at most about 0.5 copies of VIM per copy of SDHA, and wherein the composition is mostly in aggregate form, which is in suspension or pellet form. In some embodiments, a composition of cells comprising PRPs as disclosed herein, comprises, or consists of, or consists essentially of at most about 0.5% Ki67+ cells, and wherein the composition is mostly in aggregate form, which is in suspension or pellet form.

In some embodiments, a composition of cells comprising PRPs as disclosed herein, comprises, or consists of, or consists essentially of at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at most about 0.5 copies of VIM per copy of SDHA, at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at most about 0.5 copies of VIM per copy of SDHA, at least about 70% aggregate biomass, and wherein the composition is mostly in aggregate form, which is in suspension or pellet form. In some embodiments, a composition of cells comprising PRPs as disclosed herein, comprises, or consists of, or consists essentially of at most about 0.5 copies of VIM per copy of SDHA, and wherein the composition is mostly in aggregate form, which is in suspension or pellet form.

In some embodiments, a composition of cells comprising PRPs as disclosed herein, comprises, or consists of, or consists essentially of at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of at least about 70% aggregate biomass, a percentage viability of at least about 50%, and wherein the composition is mostly in aggregate form, which is in suspension or pellet form. In some embodiments, a composition of cells comprising PRPs as disclosed herein, comprises, or consists of, or consists essentially of at least about 70% aggregate biomass, and wherein the composition is mostly in aggregate form, which is in suspension or pellet form.

In some embodiments, a composition of cells comprising PRPs as disclosed herein, comprises, or consists of, or consists essentially of a percentage viability of at least about 50%, no detectable expression of LNCPRESS2 and/or AC009446.1, and wherein the composition is mostly in aggregate form, which is in suspension or pellet form. In some embodiments, a composition of cells comprising PRPs as disclosed herein, comprises, or consists of, or consists essentially of a percentage viability of at least about 50%, and wherein the composition is mostly in aggregate form, which is in suspension or pellet form.

In some embodiments, a composition of cells comprising PRPs as disclosed herein, comprises, or consists of, or consists essentially of, one or more, two or more, or three or more of the characteristics provided in Table 2.

TABLE 2
Specifications for Doses for Administration Comprising a
Composition of Cells Comprising PRPs as Disclosed Herein

| Specification | Characteristic |
|---|---|
| 1 × 10⁶ dose - Live Cell Concentration | At least 2.72 × 10⁷ and at most 4.08 × 10⁷ |
| 2 × 10⁶ dose - Live Cell Concentration | At least 5.04 × 10⁷ and at most 7.56 × 10⁷ |
| 3 × 10⁶ dose - Live Cell Concentration | At least 7.12 × 10⁷ and at most 1.07 × 10⁸ |
| 4 × 10⁶ dose - Live Cell Concentration | At least 9.44 × 10⁷ and at most 1.42 × 10⁸ |
| Endotoxin contamination levels | At most 5.0 EU/mL |
| Percentage Viability | At least 40% |

In some embodiments, a composition of cells comprising PRPs as disclosed herein, comprises, or consists of, or consists essentially of at least 2.72×10⁷ cells/mL and at most 4.08×10⁷ cells/mL, an endotoxin level that is at most 5.0 EU/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 2.72×10⁷ cells/mL and at most 4.08×10⁷ cells/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 2.72×10⁷ cells/mL and at most 4.08×10⁷ cells/mL, and wherein the composition is mostly in aggregate form, which is in suspension or pellet form.

In some embodiments, a composition of cells comprising PRPs as disclosed herein, comprises, or consists of, or consists essentially of at least 5.04×10⁷ cells/mL and at most 7.56×10⁷ cells/mL, an endotoxin level that is at most 5.0 EU/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 5.04×10⁷ cells/mL and at most 7.56×10⁷ cells/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 5.04×10⁷ cells/mL and at most 7.56×10⁷ cells/mL, and wherein the composition is mostly in aggregate form, which is in suspension or pellet form.

In some embodiments, a composition of cells comprising PRPs as disclosed herein, comprises, or consists of, or consists essentially of at least 7.12×10⁷ cells/mL and at most 1.07×10⁸ cells/mL, an endotoxin level that is at most 5.0 EU/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 7.12×10⁷ cells/mL and at most 1.07×10⁸ cells/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 7.12×10⁷ cells/mL and at most 1.07×10⁸ cells/mL, an endotoxin level that is at most 5.0 EU/mL, wherein the composition is mostly in aggregate form, which is in suspension or pellet form.

In some embodiments, a composition of cells comprising PRPs as disclosed herein, comprises, or consists of, or consists essentially of at least 9.44×10⁷ cells/mL and at most 1.42×10⁸ cells/mL, an endotoxin level that is at most 5.0 EU/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 9.44×10⁷ cells/mL and at most 1.42×10⁸ cells/mL, a percentage viability of at least 40%, and wherein the composition is mostly in aggregate form, which is in suspension or pellet form. In some embodiments, a composition of cells comprising PRPs as disclosed herein, comprises, or consists of, or consists essentially of at least 9.44×10⁷ cells/mL and at most 1.42×10⁸ cells/mL, and wherein the composition is mostly in aggregate form, which is in suspension or pellet form.

In some embodiments, a composition of cells comprising PRPs as disclosed herein, comprises, or consists of, or consists essentially of at least about 2.72×10⁷ cells/mL and at most about 4.08×10⁷ cells/mL, an endotoxin level that is at most about 5.0 EU/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 2.72×10⁷ cells/mL and at most about 4.08×10⁷ cells/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 2.72×10⁷ cells/mL and at most about 4.08×10⁷ cells/mL, and wherein the composition is mostly in aggregate form, which is in suspension or pellet form.

In some embodiments, a composition of cells comprising PRPs as disclosed herein, comprises, or consists of, or consists essentially of at least about 5.04×10⁷ cells/mL and at most about 7.56×10⁷ cells/mL, an endotoxin level that is at most about 5.0 EU/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 5.04×10⁷ cells/mL and at most about 7.56×10⁷ cells/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 5.04×10⁷ cells/mL and at most about 7.56×10⁷ cells/mL, and wherein the composition is mostly in aggregate form, which is in suspension or pellet form.

In some embodiments, a composition of cells comprising PRPs as disclosed herein, comprises, or consists of, or consists essentially of at least about 7.12×10⁷ cells/mL and at most about 1.07×10⁸ cells/mL, an endotoxin level that is at most about 5.0 EU/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 7.12×10⁷ cells/mL and at most about 1.07×10⁸ cells/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 7.12×10⁷ cells/mL and at most about 1.07×10⁸ cells/mL, an endotoxin level that is at most about 5.0 EU/mL, wherein the composition is mostly in aggregate form, which is in suspension or pellet form.

In some embodiments, a composition of cells comprising PRPs as disclosed herein, comprises, or consists of, or consists essentially of at least about 9.44×10⁷ cells/mL and at most about 1.42×10⁸ cells/mL, an endotoxin level that is at most about 5.0 EU/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 9.44×10⁷ cells/mL and at most about 1.42×10⁸ cells/mL, a percentage viability of at least about 40%, and wherein the composition is mostly in aggregate form, which is in suspension or pellet form. In some embodiments, a composition of cells comprising PRPs as disclosed herein, comprises, or consists of, or consists essentially of at least about 9.44×10⁷ cells/mL and at most about 1.42×10⁸ cells/mL, and wherein the composition is mostly in aggregate form, which is in suspension or pellet form.

In some embodiments, the composition of cells consists of or consists essentially of photoreceptor precursor cells (PRPs) and, optionally, a pharmaceutically acceptable excipient thereof, wherein the composition comprises: NR2E3+ cells and between at least about 20% to about 60% PPP4R4+ cells; wherein the PRPs comprise less than or equal to about 0.5 copies of VIM per copy of SDHA, and wherein the composition of comprises at least about 90% PRP cells. In some embodiments, the composition consists of or consists essentially of at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

In some embodiments, the median diameter of a cell aggregate is about 15 μm to about 120 μm, (e.g., about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, or about 120 μm). In some embodiments, the median diameter (e.g., particle diameter) of a cell aggregate is between about 15 μm and about 120 μm (e.g., about 15 μm to about 120 μm, about 20 μm to about 115 μm, about 35 μm to about 110 μm, about 35 μm to about 105 μm, about 40 μm to about 100 μm, about 45 μm to about 95 μm, about 50 μm to about 90 μm, about 55 μm to about 85 μm, about 60 μm to about 80 μm, about 65 μm to about 75 μm, about 30 μm to about 60 μm, about 40 μm to about 50 μm, about 20 μm to about 60 μm, about 20 μm to about 50 μm, or about 30 μm to about 60 μm). In some embodiments, the median diameter of a cell aggregate is between about 30 μm and about 60 μm. In some embodiments, the median diameter of a cell aggregate is about 50 μm. In some embodiments, the median diameter of a cell aggregate is at least about 20 μm.

In some embodiments, the composition comprising the population of PRP cells does not express LNCPRESS2 and/or AC009446.1 pluripotency markers above the limit of detection (LOD). Methods for detecting, characterizing, and/or quantitating, nucleic acid sequences; and for detecting, characterizing, and/or quantitating, mRNA expression, are known to persons skilled in the art, and include, but are not limited to, for example, PCR procedures, RT-PCR, quantitative PCR or RT-PCR, Northern blot analysis, differential gene expression, RNA protection assay, microarray analysis, hybridization methods, serial analysis of gene expression (SAGE), hybridization based on digital barcode quantification assays, multiplex RT-PCR, ddPCR, qRT-PCR, qPCR, UV spectroscopy, DNA sequencing, RNA sequencing, next-generation sequencing, including RNA-seq, lysate-based hybridization assays utilizing branched DNA signal amplification, such as the QuantiGene 2.0 Single Plex, and branched DNA analysis methods. In some embodiments, the composition comprising the population of PRP cells does not express LNCPRESS2 and/or AC009446.1 pluripotency markers above the limit of detection as measured by droplet digital polymerase chain reaction (ddPCR).

In some embodiments, ddPCR is performed using Bio-Rad's QX200™ ddPCR System. In some embodiments, the LOD is determined against a reference gene, e.g., SDHA. In some embodiments, the number of copies of VIM is determined relative to copies of a reference gene, e.g., SDHA.

In some embodiments, expression of SDHA, VIM, LNCPRESS2, and/or AC009446.1 was assessed by droplet digital PCR (ddPCR) for cDNA derived from a composition comprising the population of PRP cells. In some embodiments, sample cDNA is diluted and prepared in a PCR master mix. In some embodiments, the master mix containing diluted sample cDNA may be transferred to a 96-well plate. In some embodiments, the plate may be sealed with a ddPCR PX1 PCR Plate Sealer (Bio-Rad, Hercules, CA; Cat. No. 1814000). In some embodiments, droplets may be generated using a QX200™ Automated Droplet Generator (Bio-Rad, Hercules, CA; Cat. No. 1864101). In some embodiments, PCR may be performed to produce amplified droplets using a C1000 Touch™ Thermal Cycler (Bio-Rad, Hercules, CA; Cat. No. 1851148). In some embodiments, the amplified droplets may be assessed using a QX200™ Droplet Reader (Bio-Rad, Hercules, CA; Cat. No. 1864003) to assess VIM, LNCPRESS2, and/or AC009446.1 in channel 1 and SDHA (reference gene) in channel 2 with FAM/VIC probes. In some embodiments, copies per μL of VIM, LNCPRESS2, and/or AC009446.1 divided by copies per μL of SDHA may be reported as a ratio (copies/copy).

In some embodiments, acceptable samples contain at least 10,000 droplets. In some embodiments, acceptable samples contain a percentage coefficient of variation of less than or equal to 25%. In some embodiments, the cDNA derived from a composition comprising the population of PRP cells may contain between 800-4,000 SDHA copies per μL. In some embodiments, a cell product suitable for downstream applications will contain no more 0.50 copies/copy of VIM per SDHA (copies per μL of VIM divided by copies per μL of SDHA). In some embodiments, outliers may be assessed and removed, if appropriate, with a Dixon Q Test.

In some embodiments, a drug product comprising PRPs comprises a suspension of at least about 70% to about 100% (e.g., about 70% to about 100%, about 70% to about 90%, about 70% to about 80%, about 80% to about 100%, about 80% to about 90%, or about 90% to about 100%) induced PRP cells (iPRP). In some embodiments, a drug product comprising PRPs comprises a suspension of at least about 70%, at least about 80%, at least about 90%, at least about 95%, at least about 96%, at least about 97%, at least about 98%, at least about 99%, or about 100% induced PRP cells (iPRP). In some embodiments, the drug product may be suspended in a balanced salt solution (BSS) buffer supplemented with human serum albumin (HSA) (e.g., 0.2% HSA). In some embodiments, the drug product is in a single cell form, an aggregate form, or in a suspended form. In some embodiments, the drug product is in a suspended form. In some embodiments, the drug product is in a single cell form or an aggregate form. In some embodiments, the drug product is in a pellet formed by centrifugation of a suspension of the drug product. In some embodiments, the drug product comprises aggregates in suspension. In some embodiments, the drug product comprises aggregates in pellet form. In some embodiments, the drug product comprises aggregates that have settled to the bottom of a tube or vial. In some embodiments, the drug product comprises aggregates and single cells in suspension. In some embodiments, the drug product comprises aggregates and single cells in pellet form. In some embodiments, the drug product comprises aggregates and single cells that have settled to the bottom of a tube or vial.

In some embodiments, a percentage cell viability of a drug product comprising PRP cells in a suspended (e.g., single cell suspension) form may be calculated using automated cell counting including, for example, with acridine orange and/or propidium iodide using, for example, a Cellaca™ MX. In some embodiments, the percentage viability of the drug product comprising PRP cells in a suspended form may be calculated to be at least about 50% (e.g., about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the drug product comprising PRP cells in a suspended form may be calculated to be at least about 50% to at least about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the drug product comprising PRP cells in a suspended form may be calculated to be at least about 50%.

In some embodiments, a percentage cell viability of a drug product comprising PRP cells in a suspended form may be calculated using automated cell counting including, for example, with an IncuCyte® Live-Cell Analysis System (Sartorius; Göttingen, Germany; Cat. No. SX5). In some embodiments, the percentage viability of the drug product comprising PRP cells in a suspended form may be calculated to be at least about 50% (e.g., about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the drug product comprising PRP cells in a suspended form may be calculated to be at least about 50% to at least about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the drug product comprising PRP cells in a suspended form may be calculated to be at least about 50%.

In some embodiments, a percentage cell viability of a drug product comprising PRP cells in a single cell form or an aggregate form may be calculated using automated cell counting including, for example, with acridine orange and/or propidium iodide using, for example, a Cellaca™ MX. In some embodiments, the percentage viability of the drug product comprising PRP cells in a single cell form or an aggregate form may be calculated to be at least about 50% (e.g., about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the drug product comprising PRP cells in a single cell form or an aggregate form may be calculated to be at least about 50% to at least about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the drug product comprising PRP cells in a single cell form or an aggregate form may be calculated to be at least about 50%.

In some embodiments, a percentage cell viability of a drug product comprising PRP cells in a single cell form or an aggregate form may be calculated using automated cell counting including, for example, with an IncuCyte® Live-Cell Analysis System (Sartorius; Göttingen, Germany; Cat. No. SX5). In some embodiments, the percentage viability of the drug product comprising PRP cells may be calculated to be at least about 50% (e.g., about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the drug product comprising PRP cells in a single cell form or an aggregate form may be calculated to be at least about 50% to at least about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the drug product comprising PRP cells in a single cell form or an aggregate form may be calculated to be at least about 50%.

In some embodiments, a percentage cell viability of a dose for administration comprising PRP cells in a suspended form may be calculated using automated cell counting including, for example, with acridine orange and/or propidium iodide using, for example, a Cellaca™ MX. In some embodiments, the percentage viability of the dose for administration comprising PRP cells in a suspended form may be calculated to be at least about 40% (e.g., about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the dose for administration comprising PRP cells in a suspended form may be calculated to be at least about 40% to at least about 100% (e.g., about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the dose for administration comprising PRP cells in a suspended form may be calculated to be at least about 40%.

In some embodiments, a percentage cell viability of a dose for administration comprising PRP cells in a suspended form may be calculated using automated cell counting including, for example, with an IncuCyte® Live-Cell Analysis System (Sartorius; Göttingen, Germany; Cat. No. SX5). In some embodiments, the percentage viability of the dose for administration comprising PRP cells in a suspended form may be calculated to be at least about 40% (e.g., about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, the percentage viability of the dose for administration comprising PRP cells in a suspended form may be calculated to be at least about 40% to at least about 100% (e.g., about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the dose for administration comprising PRP cells in a suspended form may be calculated to be at least about 40%.

In some embodiments, the percentage viability of the dose for administration comprising PRP cells in a single cell form or an aggregate form may be calculated to be at least about 40% (e.g., about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%). In some embodiments, a percentage cell viability of a dose for administration comprising PRP cells in a single cell form or an aggregate form may be calculated using automated cell counting including, for example, with an IncuCyte® Live-Cell Analysis System (Sartorius; Göttingen, Germany; Cat. No. SX5). In some embodiments, the percentage viability of the dose for administration comprising PRP cells in a single cell form or an aggregate form may be calculated to be at least about 40% to at least about 100% (e.g., about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the percentage viability of the dose for administration comprising PRP cells in a single cell form or an aggregate form may be calculated to be at least about 40%.

In some embodiments, the dose for administration comprises aggregates in suspension. In some embodiments, the dose for administration comprises aggregates in pellet form. In some embodiments, the dose for administration comprises aggregates that have settled to the bottom of a tube or vial. In some embodiments, the dose for administration comprises aggregates and single cells in suspension. In some embodiments, the dose for administration comprises aggregates and single cells in pellet form. In some embodiments, the dose for administration comprises aggregates and single cells that have settled to the bottom of a tube or vial. In some embodiments, the dose for administration is made by the methods disclosed in WO 2024/073759 A1, which is incorporated herein by reference in its entirety.

### Methods of Qualifying a Composition of Cells for Treatment of an Eye Disease

A composition of cells disclosed herein may be assessed for suitability (e.g., qualified for use as a therapeutic) for an eye (e.g., ocular) disease or disorder, such as a retinal disease or disorder. In some embodiments, the eye disease or disorder and/or the retinal disease or disorder may be characterized by loss of photoreceptor cells (e.g., loss of rod cells and/or cone cells) and/or a loss of photoceptor cell function. In some embodiments, the eye disease or disorder and/or the retinal disease may include, for example, a primary photoreceptor disease. In some embodiments, the primary photoreceptor disease may include, for example, retinitis pigmentosa, cone-rod disease, rod-cone disease, cone dystrophy, and cone-rod dystrophy. In some embodiments, the primary photoreceptor disease is an inherited retinal disorder. In some embodiments the inherited retinal disorder may be, for example, Usher Syndrome. As used herein, the term “suitable” may mean that a cell population is suitable for administration, and/or contains the correct proportions of cells, and/or the cells are functional, and/or the cell population is subsequently therapeutically effective after administration.

In an aspect, provided herein is a method of identifying a suitable cell population for treatment of an eye disease, the method comprising obtaining a population of PRPs, measuring a percentage of PPP4R4+ cells in a cell population, and measuring a copy number of VIM per copy SDHA, wherein the cell population is identified as a suitable cell population for treatment of an eye disease when the percentage of PPP4R4+ cells in the cell population is between about 20% and about 60%, and the copy number of VIM per copy of SDHA is less than or equal to about 0.5. In some embodiments, the method further comprises measuring a percentage of NR2E3+, AIPL1+, RCVRN+, PAX6+, CHX10+, TYRP1+; and/or Ki67+ cells in the cell population, wherein the cell population is identified as a suitable cell population for treatment of an eye disease when a substantial portion of the cells are NR2E3+, the percentage of AIPL1+ cells is least about 90%, the percentage of RCVRN+ cells is least about 90%, the percentage of PAX6+ cells is no more than about 7%, the percentage of CHX10+ cells is no more than about 7%, the percentage of TYRP1+ cells is no more than about 0.5%, and the percentage of Ki67+ cells is no more than about 0.5%. In some embodiments, when the cell population comprises between at least about 20% to about 60% PPP4R4+ cells, and wherein and the cells comprise less than or equal to 0.5 copies of VIM per copy of SDHA, then the cells are identified as suitable for the treatment.

In some embodiments, the method further comprises measuring a percentage biomass of cell aggregates in the cell composition, wherein the cell population is identified as a suitable cell population for treatment of an eye disease or disorder when the cell composition comprises at least 70% biomass of cell aggregates. In some embodiments, the method further comprises measuring a median diameter (e.g., particle diameter) of cell aggregates in the cell composition, wherein the cell population is identified as a suitable cell population for treatment of an eye disease or disorder when the cell aggregates are observed to have a median diameter of at least about 30 μm to at least about 60 μm.

In some embodiments, the method further comprises measuring a percentage viability of the cell composition, wherein the cell population is identified as a suitable cell population for treatment of an eye disease or disorder when the cell composition is observed to have a percentage viability of at least about 50%. In some embodiments, the method further comprises measuring a percentage viability of the cell composition, wherein the cell population is identified as a suitable cell population for treatment of an eye disease or disorder when the cell composition is observed to have a percentage viability of at least about 40%.

In some embodiments, a percentage of PPP4R4+, AIPL1+, RCVRN+, PAX6+, CHX10+, TYRP1+, or Ki67+ cells is determined by flow cytometry. In some embodiments, copies of VIM per copy of SDHA is determined by ddPCR. In some embodiments, ddPCR is performed using Bio-Rad's QX200™ ddPCR System. In some embodiments, the copies of VIM are determined relative to reference gene SDHA. In some embodiments, the percentage biomass of aggregates and/or medium diameter of cell aggregates in the cell composition is quantified using a particle size analyzer. In some embodiments, the percentage viability of a cell population is determined by an automated cell counter using cell stains, including, for example, acridine orange and/or propidium iodine.

In some embodiments, the cell population is derived from PSCs or PSC-derived cells, optionally iPSCs. In some embodiments, the cell population is of human origin. In some embodiments, the cell population is suitable for grafting to a subretinal space of a subject's eye.

In particular embodiments, the cell population is identified as a suitable cell population for treatment of an eye disease when the percentage of RCVRN+ cells in the cell population is at least about 80% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100%). In some embodiments, the cell population is identified as a suitable cell population (e.g., qualified for use as a therapeutic) for treatment of an eye disease when the percentage of RCVRN+ cells in the cell population is about 80% to about 100% (e.g., about 80% to about 100%, about 80% to about 90%, about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%). In some embodiments, the cell population is identified as a suitable cell population for treatment of an eye disease when the percentage of RCVRN+ cells in the cell population is at least about 90% of RCVRN expressing cells.

In particular embodiments, the cell population is identified as a suitable cell population for treatment of an eye disease (e.g., qualified for use as a therapeutic) when the percentage of AIPL1+ cells in the cell population is at least about 80% (e.g., about 80%, about 81%, about 82%, about 83%, about 84%, about 85%, about 86%, about 87%, about 88%, about 89% about 90%, about 91%, about 92%, about 93%, about 94%, about 95% about 96%, about 97%, about 98%, about 99%, or about 100%). In some embodiments, the cell population is identified as a suitable cell population (e.g., qualified for use as a therapeutic) for treatment of an eye disease when the percentage of AIPL1+ cells in the cell population is about 80% to about 100% (e.g., about 80% to about 100%, about 80% to about 90%, about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%). In some embodiments, the cell population is identified as a suitable cell population for treatment of an eye disease when the percentage of RCVRN+ cells in the cell population is at least about 90% of AIPL1-expressing cells.

In some In some embodiments, the cell population is identified as a suitable cell population for treatment of an eye disease (e.g., qualified for use as a therapeutic) when the percentage of PPP4R4+ cells in the cell population is at least about 20% (e.g., about 20%, about 21%, at least 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%). In some embodiments, the cell population is identified as a suitable cell population for treatment of an eye disease when the percentage of PPP4R4+ cells in the cell population is at most 60% (e.g., about 20%, about 21%, at least 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%. embodiments, the cell population is identified as a suitable cell population (e.g., qualified for use as a therapeutic) for treatment of an eye disease when the percentage of PPP4R4+ cells in the cell population is between about 20% and about 60% (e.g., about 20% to about 60%, about 20% to about 50%, about 20% to about 40%, about 20% to about 30%, about 30% to about 60%, about 30% to about 50%, about 30% to about 40%, about 40% to about 60%, about 40% to about 50%, or about 50% to about 60%). In some embodiments, the cell population is identified as a suitable cell population for treatment of an eye disease when the percentage of PPP4R4+ cells in the cell population is at least about 20%. In some embodiments, the cell population is identified as a suitable cell population for treatment of an eye disease when the percentage of PPP4R4+ cells in the cell population is at most about 60%. In some embodiments, the cell population is identified as a suitable cell population for treatment of an eye disease when the percentage of PPP4R4+ cells in the cell population is at least about 20% and at most about 60%.

In some embodiments, the cell population is identified as a suitable cell population for treatment of an eye disease (e.g., qualified for use as a therapeutic) when the percentage of NR2E3+ cells in the cell population is at least about 10% to at most about 100% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%). In some embodiments, the cell population is identified as a suitable cell population (e.g., qualified for use as a therapeutic) for treatment of an eye disease when the percentage of NR2E3+ cells in the cell population is between about 10% and about 100% (e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%). In some embodiments, the cell population is identified as a suitable cell population for treatment of an eye disease when the percentage of NR2E3+ cells in the cell population is at least about 25% to at most about 65% NR2E3-expressing cells. In some embodiments, the cell population is identified as a suitable cell population for treatment of an eye disease when the percentage of NR2E3+ cells in the cell population is at least about 40% to at most about 80% NR2E3-expressing cells.

In some embodiments, the cell population is identified as a suitable cell population for treatment of an eye disease when a composition comprising the population of cells comprising 90% PRP cells comprises at least about 90% AIPL1-expressing cells and at least about 90% RCVRN-expressing cells. In further embodiments, the cell population is identified as a suitable cell population for treatment of an eye disease when, of those 90% PRPs expressing AIPL1 and RCVRN, a first portion will express PPP4R4 and a second portion will express NR2E3. In further embodiments, In further embodiments, the cell population is identified as a suitable cell population for treatment of an eye disease when, of those 90% PRPs expressing AIPL1 and RCVRN, the first portion will express PPP4R4 but will not express NR2E3. In further embodiments, the cell population is identified as a suitable cell population for treatment of an eye disease when, of those 90% PRPs expressing AIPL1 and RCVRN, the second portion will express NR2E3 but will not express PPP4R4. In further embodiments, the cell population is identified as a suitable cell population for treatment of an eye disease when, of those 90% PRPs expressing AIPL1 and RCVRN, at least about 20% of the cells will express PPP4R4, at least about 80% of the cells will express NR2E3. In further embodiments, the cell population is identified as a suitable cell population for treatment of an eye disease when, of those 90% PRPs expressing AIPL1 and RCVRN, at least about 30% of the cells will express PPP4R4, at least about 70% of the cells will express NR2E3. In further embodiments, the cell population is identified as a suitable cell population for treatment of an eye disease when, of those 90% PRPs expressing AIPL1 and RCVRN, at least about 40% of the cells will express PPP4R4, at least about 60% of the cells will express NR2E3. In further embodiments, the cell population is identified as a suitable cell population for treatment of an eye disease when, of those 90% PRPs expressing AIPL1 and RCVRN, at least about 50% of the cells will express PPP4R4, at least about 50% of the cells will express NR2E3. In further embodiments, the cell population is identified as a suitable cell population for treatment of an eye disease when, of those 90% PRPs expressing AIPL1 and RCVRN, at least about 60% of the cells will express PPP4R4, at least about 40% of the cells will express NR2E3.

In some embodiments, the cell population is identified as a suitable cell population for treatment of an eye disease when the copies of VIM per copy of SDHA of cells in the cell population is about 0.0 to at most 0.5 copies as measured by ddPCR (e.g., about 0.0, about 0.05, about 0.10, about 0.15, about 0.20, about 0.25, about 0.30, about 0.35, about 0.40, about 0.45, or about 0.50 copies as measured by ddPCR). In some embodiments, the cell population is identified as a suitable cell population (e.g., qualified for use as a therapeutic) for treatment of an eye disease when the copies of VIM per copy of SDHA of cells in the cell population is between about 0.0 copies and about 0.50 copies (e.g., about 0.0 copies to about 0.1, about 0.0 copies to about 0.2, about 0.0 copies to about 0.3, about 0.0 copies to about 0.4, about 0.0 copies to about 0.5, about 0.1 copies to about 0.2, about 0.1 copies to about 0.3, about 0.1 copies to about 0.4, about 0.1 copies to about 0.5, about 0.2 copies to about 0.3, about 0.2 copies to about 0.4, about 0.2 copies to about 0.5, about 0.3 copies to about 0.4, about 0.3 copies to about 0.5, or about 0.4 copies to about 0.5), as measured by ddPCR In some embodiments, the cell population is identified as a suitable cell population for treatment of an eye disease when the copies of VIM per copy of SDHA of cells in the cell population is at most 0.5 as measured by ddPCR.

In some embodiments, the cell population is identified as a suitable cell population for treatment of an eye disease (e.g., qualified for use as a therapeutic) when the percentage of PAX6+ cells in the cell population is at most about 7.0% (e.g., about 7.0%, about 6.75%, about 6.50%, about 6.25%, about 6.0%, about 5.75%, about 5.50%, about 5.25%, about 5.0%, about 4.75%, about 4.50%, about 4.25%, about 4.0%, about 3.75%, about 3.50%, about 3.25%, about 3.0%, about 2.75%, about 2.50%, about 2.25%, about 2.0%, about 1.75%, about 1.50%, about 1.25%, about 1.0%, about 0.75%, about 0.50%, about 0.25%, or about 0.0%). In some embodiments, the cell population is identified as a suitable cell population (e.g., qualified for use as a therapeutic) for treatment of an eye disease when the percentage of PAX6+ cells in the cell population is between about 0% and about 7% (e.g., about 0% to about 7%, about 0% to about 6%, about 0% to about 5%, about 0% to about 4%, about 0% to about 3%, about 0% to about 2%, about 0% to about 1%, about 1% to about 7%, about 1% to about 6%, about 1% to about 5%, about 1% to about 4%, about 1% to about 3%, about 1% to about 2%, about 2% to about 7%, about 2% to about 6%, about 2% to about 5%, about 2% to about 4%, about 2% to about 3%, about 3% to about 7%, about 3% to about 6%, about 3% to about 5%, about 3% to about 4%, about 4% to about 7%, about 4% to about 6%, about 4% to about 5%, about 5% to about 7%, about 5% to about 6%, or about 6% to about 7%). In some embodiments, the cell population is identified as a suitable cell population for treatment of an eye disease when the percentage of PAX6+ cells in the cell population is at most about 7%.

In some embodiments, the cell population is identified as a suitable cell population (e.g., qualified for use as a therapeutic) for treatment of an eye disease when the percentage of CHX10+ cells in the cell population is at most about 7.0% (e.g., about 7.0%, about 6.75%, about 6.50%, about 6.25%, about 6.0%, about 5.75%, about 5.50%, about 5.25%, about 5.0%, about 4.75%, about 4.50%, about 4.25%, about 4.0%, about 3.75%, about 3.50%, about 3.25%, about 3.0%, about 2.75%, about 2.50%, about 2.25%, about 2.0%, about 1.75%, about 1.50%, about 1.25%, about 1.0%, about 0.75%, about 0.50%, about 0.25%, or about 0.0%). In some embodiments, the cell population is identified as a suitable cell population (e.g., qualified for use as a therapeutic) for treatment of an eye disease when the percentage of CHX10+ cells in the cell population is between about 0% and about 7% (e.g., about 0% to about 7%, about 0% to about 6%, about 0% to about 5%, about 0% to about 4%, about 0% to about 3%, about 0% to about 2%, about 0% to about 1%, about 1% to about 7%, about 1% to about 6%, about 1% to about 5%, about 1% to about 4%, about 1% to about 3%, about 1% to about 2%, about 2% to about 7%, about 2% to about 6%, about 2% to about 5%, about 2% to about 4%, about 2% to about 3%, about 3% to about 7%, about 3% to about 6%, about 3% to about 5%, about 3% to about 4%, about 4% to about 7%, about 4% to about 6%, about 4% to about 5%, about 5% to about 7%, about 5% to about 6%, or about 6% to about 7%). In some embodiments, the cell population is identified as a suitable cell population for treatment of an eye disease when the percentage of CHX10+ cells in the cell population is at most about 7%.

In some embodiments, the cell population is identified as a suitable cell population (e.g., qualified for use as a therapeutic) for treatment of an eye disease when the percentage of TYRP1+ cells in the cell population is at most about 0.5% (e.g., about 0.00%, about 0.05%, about 0.10%, about 0.15%, about 0.20%, about 0.25%, about 0.30%, about 0.35%, about 0.40%, about 0.45%, or about 0.50%). In some embodiments, the cell population is identified as a suitable cell population (e.g., qualified for use as a therapeutic) for treatment of an eye disease when the percentage of TYRP1+ cells in the cell population is between about 0% and about 0.5% (e.g., about 0.0% to about 0.1%, about 0.0% to about 0.2%, about 0.0% to about 0.3%, about 0.0% to about 0.4%, about 0.0% to about 0.5%, about 0.1% to about 0.2%, about 0.1% to about 0.3%, about 0.1% to about 0.4%, about 0.1% to about 0.5%, about 0.2% to about 0.3%, about 0.2% to about 0.4%, about 0.2% to about 0.5%, about 0.3% to about 0.4%, about 0.3% to about 0.5%, or about 0.4% to about 0.5%). In some embodiments, the cell population is identified as a suitable cell population for treatment of an eye disease when the percentage of TYRP1+ cells in the cell population is at most 0.5%. In some embodiments, the cell population is identified as a suitable cell population for treatment of an eye disease when expression of TYRP1+ cells is not detectable.

In some embodiments, the cell population is identified as a suitable cell population (e.g., qualified for use as a therapeutic) for treatment of an eye disease when the percentage of Ki67+ cells in the cell population is at most about 0.5% (e.g., about 0.00%, about 0.05%, about 0.10%, about 0.15%, about 0.20%, about 0.25%, about 0.30%, about 0.35%, about 0.40%, about 0.45%, or about 0.50%. In some embodiments, the cell population is identified as a suitable cell population (e.g., qualified for use as a therapeutic) for treatment of an eye disease when the percentage of Ki67+ cells in the cell population is between about 0% and about 0.5% (e.g., about 0.0% to about 0.1%, about 0.0% to about 0.2%, about 0.0% to about 0.3%, about 0.0% to about 0.4%, about 0.0% to about 0.5%, about 0.1% to about 0.2%, about 0.1% to about 0.3%, about 0.1% to about 0.4%, about 0.1% to about 0.5%, about 0.2% to about 0.3%, about 0.2% to about 0.4%, about 0.2% to about 0.5%, about 0.3% to about 0.4%, about 0.3% to about 0.5%, or about 0.4% to about 0.5%). In some embodiments, the cell population is identified as a suitable cell population for treatment of an eye disease when the percentage of Ki67+ cells in the cell population is at most 0.5%. In some embodiments, the cell population is identified as a suitable cell population for treatment of an eye disease when expression of Ki67+ cells is not detectable.

In some embodiments, the cell population is identified as a suitable cell population (e.g., qualified for use as a therapeutic) for treatment of an eye disease when the composition consists of or consists essentially of: NR2E3+ cells and between at least about 20% to about 60% PPP4R4+ cells; wherein the PRPs comprise less than or equal to about 0.5 copies of VIM per copy of SDHA, and wherein the composition comprises at least about 90% PRP cells. In some embodiments the cell population is identified as a suitable cell population (e.g., qualified for use as a therapeutic) for treatment of an eye disease when the composition consists of or consists essentially of at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

In some embodiments, the cell population is identified as a suitable cell population (e.g., qualified for use as a therapeutic) for treatment of an eye disease when the cell population comprises substantially NR2E3+ cells, between at least about 20% and at most about 60% PPP4R4+ cells, at most 0.5 copies VIM per copy SDHA, at least about 90% AIPL1+ cells, at least about 90% RCVRN+ cells, at most about 7% PAX6+ cells, at most about 7% CHX10+ cells, at most about 0.5% TYRP1+ cells, and/or at most about 0.5% Ki67+ cells. In some embodiments, the cell population is identified as a suitable cell population for treatment of an eye disease when the cell population comprises between at least about 20% and at most about 60% PPP4R4+ cells and at most 0.5 copies VIM per copy SDHA. In some embodiments, the cell population is identified as a suitable cell population for treatment of an eye disease when the cell population comprises NR2E3+ cells and between at least about 20% and at most about 60% PPP4R4+ cells. In some embodiments, the cell population is identified as a suitable cell population for treatment of an eye disease when the cell population comprises NR2E3+ cells, between at least about 20% and at most about 60% PPP4R4+ cells, and at most 0.5 copies VIM per copy SDHA. In some embodiments, the cell population is identified as a suitable cell population for treatment of an eye disease when the cell population comprises at least about 90% AIPL1+ cells and at least about 90% RCVRN+ cells. In some embodiments, the cell population is identified as a suitable cell population for treatment of an eye disease when the cell population comprises NR2E3+ cells, between at least about 20% and at most about 60% PPP4R4+ cells, at most 0.5 copies VIM per copy SDHA, at least about 90% AIPL1+ cells, and at least about 90% RCVRN+ cells. In some embodiments, the cell population is identified as a suitable cell population for treatment of an eye disease when the cell population comprises at most about 7% PAX6+ cells, at most about 7% CHX10+ cells, at most about 0.5% TYRP1+ cells, and/or at most about 0.5% Ki67+ cells.

In some embodiments, the cell population is identified as a suitable cell population for treatment of an eye disease (e.g., a disease in which there has been or is a risk of loss of photoreceptors) when the cell population comprises, or consists of, or consists essentially of, one or more, two or more, three or more, four or more, five or more, six or more, seven or more, eight or more, nine or more, ten or more, eleven or more, or twelve of the characteristics provided in Table 3.

TABLE 3
Specifications for Drug Products Comprising a Composition
of Cells Comprising PRPs as Disclosed Herein

| Specification | Characteristic |
|---|---|
| AIPL1+ | At least 90% |
| RCVRN+ | At least 90% |
| PPP4R4+ | At least 20% and at most 60% |
| NR2E3+ | Detectable |
| PAX6+ | At most 7% |
| CHX10+ | At most 7% |
| TYRP1+ | At most 0.5% |
| Ki67+ | At most 0.5% |
| Copies of VIM per copy of SDHA | At most 0.5 copies/copy |
| Aggregate biomass | At least 70% |
| Percentage Viability | At least 50% |
| LNCPRESS2 and AC009446.1 | Not detectable |

In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, and wherein the composition is mostly in aggregate form, which is in suspension or pellet form.

In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, at least 20% and at most 60% PPP4R4+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% RCVRN+ cells, and wherein the composition is mostly in aggregate form, which is in suspension or pellet form.

In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, at most 7% PAX6+ cells, at most 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 90% AIPL1+ cells, at least 90% RCVRN+ cells, at least 20% and at most 60% PPP4R4+ cells, NR2E3+ cells, at most 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least 20% and at most 60% PPP4R4+ cells, and wherein the composition is mostly in aggregate form, which is in suspension or pellet form.

In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, at most 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, at most 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 7% PAX6+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, at most 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 7% CHX10+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most 0.5% TYRP1+ cells, at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% TYRP1+ cells, at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% TYRP1+ cells, at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% TYRP1+ cells, at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% TYRP1+ cells, at most 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% TYRP1+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most 0.5% Ki67+ cells, at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% Ki67+ cells, at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% Ki67+ cells, at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% Ki67+ cells, at most 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most 0.5% Ki67+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most 0.5 copies of VIM per copy of SDHA, at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most 0.5 copies of VIM per copy of SDHA, at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at most 0.5 copies of VIM per copy of SDHA, at least 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most 0.5 copies of VIM per copy of SDHA, and wherein the composition is mostly in aggregate form, which is in suspension or pellet form.

In some embodiments, the cell population as defined herein is identified as suitable for treatment of an eye disease when the population comprises, or consists of, or consists essentially of at least 70% aggregate biomass, a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least 70% aggregate biomass, a percentage viability of at least 50%, and wherein the composition is mostly in aggregate form, which is in suspension or pellet form. In some embodiments, the cell population as defined herein is identified as suitable for treatment of an eye disease when the population comprises, or consists of, or consists essentially of at least 70% aggregate biomass, and wherein the composition is mostly in aggregate form, which is in suspension or pellet form.

In some embodiments, the cell population as defined herein is identified as suitable for treatment of an eye disease when the population comprises, or consists of, or consists essentially of a percentage viability of at least 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of a percentage viability of at least 50%, and wherein the composition is mostly in aggregate form, which is in suspension or pellet form.

In some embodiments, the cell population as defined herein is identified as suitable for treatment of an eye disease when the population comprises, or consists of, or consists essentially of no detectable expression of LNCPRESS2 and/or AC009446.1, and wherein the composition is mostly in aggregate form, which is in suspension or pellet form.

In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, and wherein the composition is mostly in aggregate form, which is in suspension or pellet form.

In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, at least about 20% and at most about 60% PPP4R4+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% RCVRN+ cells, and wherein the composition is mostly in aggregate form, which is in suspension or pellet form.

In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, at most about 7% PAX6+ cells, at most about 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 90% AIPL1+ cells, at least about 90% RCVRN+ cells, at least about 20% and at most about 60% PPP4R4+ cells, NR2E3+ cells, at most about 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, NR2E3+ cells, and wherein the composition is mostly in aggregate form, which is in suspension or pellet form. In some embodiments, the cell population as defined herein is identified as suitable for treatment of an eye disease when the population comprises, or consists of, or consists essentially of at least about 20% and at most about 60% PPP4R4+ cells, and wherein the composition is mostly in aggregate form, which is in suspension or pellet form.

In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, at most about 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, at most about 7% PAX6+ cells, and wherein the composition is mostly in aggregate form, which is in suspension or pellet form. In some embodiments, the cell population as defined herein is identified as suitable for treatment of an eye disease when the population comprises, or consists of, or consists essentially of NR2E3+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, at most about 7% CHX10+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 7% PAX6+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, at most about 0.5% TYRP1+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 7% CHX10+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most about 0.5% TYRP1+ cells, at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% TYRP1+ cells, at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% TYRP1+ cells, at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% TYRP1+ cells, at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% TYRP1+ cells, at most about 0.5% Ki67+ cells, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% TYRP1+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most about 0.5% Ki67+ cells, at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% Ki67+ cells, at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% Ki67+ cells, at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% Ki67+ cells, at most about 0.5 copies of VIM per copy of SDHA, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5% Ki67+ cells, and wherein the composition is mostly in aggregate form, which is in suspension or pellet form.

In some embodiments, the cell population as defined herein is identified as suitable for treatment of an eye disease when the population comprises, or consists of, or consists essentially of at most about 0.5 copies of VIM per copy of SDHA, at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5 copies of VIM per copy of SDHA, at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5 copies of VIM per copy of SDHA, at least about 70% aggregate biomass, and wherein the composition is mostly in aggregate form, which is in suspension or pellet form. In some embodiments, the cell population as defined herein is identified as suitable for treatment of an eye disease when the population comprises, or consists of, or consists essentially of at most about 0.5 copies of VIM per copy of SDHA, and wherein the composition is mostly in aggregate form, which is in suspension or pellet form.

In some embodiments, the cell population as defined herein is identified as suitable for treatment of an eye disease when the population comprises, or consists of, or consists essentially of at least about 70% aggregate biomass, a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of at least about 70% aggregate biomass, a percentage viability of at least about 50%, and wherein the composition is mostly in aggregate form, which is in suspension or pellet form. In some embodiments, the cell population as defined herein is identified as suitable for treatment of an eye disease when the population comprises, or consists of, or consists essentially of at least about 70% aggregate biomass, and wherein the composition is mostly in aggregate form, which is in suspension or pellet form.

In some embodiments, the cell population as defined herein is identified as suitable for treatment of an eye disease when the population comprises, or consists of, or consists essentially of a percentage viability of at least about 50%, no detectable expression of LNCPRESS2 and/or AC009446.1, and wherein the composition is mostly in aggregate form, which is in suspension or pellet form. In some embodiments, the cell population as defined herein is identified as suitable for treatment of an eye disease when the population comprises, or consists of, or consists essentially of a percentage viability of at least about 50%, and wherein the composition is mostly in aggregate form, which is in suspension or pellet form.

In some embodiments, the cell population is identified as a suitable cell population for treatment of an eye disease when the cell population comprises, or consists of, or consists essentially of, one or more, two or more, or three or more of the characteristics provided in Table 4.

TABLE 4
Specifications for Doses for Administration Comprising a
Composition of Cells Comprising PRPs as Disclosed Herein

| Specification | Characteristic |
|---|---|
| 1 × 10⁶ dose - Live Cell Concentration | At least 2.72 × 10⁷ and at most 4.08 × 10⁷ |
| 2 × 10⁶ dose - Live Cell Concentration | At least 5.04 × 10⁷ and at most 7.56 × 10⁷ |
| 3 × 10⁶ dose - Live Cell Concentration | At least 7.12 × 10⁷ and at most 1.07 × 10⁸ |
| 4 × 10⁶ dose - Live Cell Concentration | At least 9.44 × 10⁷ and at most 1.42 × 10⁸ |
| Endotoxin contamination levels | At most 5.0 EU/mL |
| Percentage Viability | At least 40% |

In some embodiments, the cell population as defined herein is identified as suitable for treatment of an eye disease when the population comprises, or consists of, or consists essentially of at least 2.72×10⁷ cells/mL and at most 4.08×10⁷ cells/mL, an endotoxin level that is at most 5.0 EU/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 2.72×10⁷ cells/mL and at most 4.08×10⁷ cells/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 2.72×10⁷ cells/mL and at most 4.08×10⁷ cells/mL, and wherein the composition is mostly in aggregate form, which is in suspension or pellet form.

In some embodiments, the cell population as defined herein is identified as suitable for treatment of an eye disease when the population comprises, or consists of, or consists essentially of at least 5.04×10⁷ cells/mL and at most 7.56×10⁷ cells/mL, an endotoxin level that is at most 5.0 EU/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 5.04×10⁷ cells/mL and at most 7.56×10⁷ cells/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 5.04×10⁷ cells/mL and at most 7.56×10⁷ cells/mL, and wherein the composition is mostly in aggregate form, which is in suspension or pellet form.

In some embodiments, the cell population as defined herein is identified as suitable for treatment of an eye disease when the population comprises, or consists of, or consists essentially of at least 7.12×10⁷ cells/mL and at most 1.07×10⁸ cells/mL, an endotoxin level that is at most 5.0 EU/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 7.12×10⁷ cells/mL and at most 1.07×10⁸ cells/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 7.12×10⁷ cells/mL and at most 1.07×10⁸ cells/mL, an endotoxin level that is at most 5.0 EU/mL, wherein the composition is mostly in aggregate form, which is in suspension or pellet form.

In some embodiments, the cell population as defined herein is identified as suitable for treatment of an eye disease when the population comprises, or consists of, or consists essentially of at least 9.44×10⁷ cells/mL and at most 1.42×10⁸ cells/mL, an endotoxin level that is at most 5.0 EU/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 9.44×10⁷ cells/mL and at most 1.42×10⁸ cells/mL, a percentage viability of at least 40%, and wherein the composition is mostly in aggregate form, which is in suspension or pellet form. In some embodiments, the cell population as defined herein is identified as suitable for treatment of an eye disease when the population comprises, or consists of, or consists essentially of at least 9.44×10⁷ cells/mL and at most 1.42×10⁸ cells/mL, and wherein the composition is mostly in aggregate form, which is in suspension or pellet form.

In some embodiments, the cell population as defined herein is identified as suitable for treatment of an eye disease when the population comprises, or consists of, or consists essentially of at least about 2.72×10⁷ cells/mL and at most about 4.08×10⁷ cells/mL, an endotoxin level that is at most about 5.0 EU/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 2.72×10⁷ cells/mL and at most about 4.08×10⁷ cells/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 2.72×10⁷ cells/mL and at most about 4.08×10⁷ cells/mL, and wherein the composition is mostly in aggregate form, which is in suspension or pellet form.

In some embodiments, the cell population as defined herein is identified as suitable for treatment of an eye disease when the population comprises, or consists of, or consists essentially of at least about 5.04×10⁷ cells/mL and at most about 7.56×10⁷ cells/mL, an endotoxin level that is at most about 5.0 EU/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 5.04×10⁷ cells/mL and at most about 7.56×10⁷ cells/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 5.04×10⁷ cells/mL and at most about 7.56×10⁷ cells/mL, and wherein the composition is mostly in aggregate form, which is in suspension or pellet form.

In some embodiments, the cell population as defined herein is identified as suitable for treatment of an eye disease when the population comprises, or consists of, or consists essentially of at least about 7.12×10⁷ cells/mL and at most about 1.07×10⁸ cells/mL, an endotoxin level that is at most about 5.0 EU/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 7.12×10⁷ cells/mL and at most about 1.07×10⁸ cells/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 7.12×10⁷ cells/mL and at most about 1.07×10⁸ cells/mL, an endotoxin level that is at most about 5.0 EU/mL, wherein the composition is mostly in aggregate form, which is in suspension or pellet form.

In some embodiments, the cell population as defined herein is identified as suitable for treatment of an eye disease when the population comprises, or consists of, or consists essentially of at least about 9.44×10⁷ cells/mL and at most about 1.42×10⁸ cells/mL, an endotoxin level that is at most about 5.0 EU/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 9.44×10⁷ cells/mL and at most about 1.42×10⁸ cells/mL, a percentage viability of at least about 40%, and wherein the composition is mostly in aggregate form, which is in suspension or pellet form. In some embodiments, the cell population as defined herein is identified as suitable for treatment of an eye disease when the population comprises, or consists of, or consists essentially of at least about 9.44×10⁷ cells/mL and at most about 1.42×10⁸ cells/mL, and wherein the composition is mostly in aggregate form, which is in suspension or pellet form.

In certain aspects, the cell population is identified as a suitable cell population (e.g., qualified for use as a therapeutic) for treatment of an eye disease when the cell population wherein the cell population does not express or has substantially no expression of CRALBP, BEST1, IGFBP5, COL8A1, ELN, UPK3B, TPM2, PTGDS, SERPINE3, TIMP3, MITF, and/or PMEL17. In some embodiments, the cell population is identified as a suitable cell population for treatment of an eye disease when the cell population has low or substantially no expression of PAX6, CHX10, Ki67, TYRP1, and/or ONECUT1. In particular embodiments, the cell population is identified as a suitable cell population for treatment of an eye disease when the cell population comprises at most about 15% (e.g., about 0% to about 15%, about 0% to about 14%, about 0% to about 13%, about 0% to about 12%, about 0% to about 11%, about 0% to about 10%, about 0% to about 9%, about 0% to about 8%, about 0% to about 7%, about 0% to about 6%, about 0% to about 5%, about 0% to about 4%, about 0% to about 3%, about 0% to about 2%, about 0% to about 1%, about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 15%, about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, about 2% to about 4%, about 2% to about 3%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6%, about 3% to about 5%, about 3% to about 4%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 4% to about 5%, about 5% to about 15%, about 5% to about 14%, about 5% to about 13%, about 5% to about 12%, about 5% to about 11%, about 5% to about 10%, about 5% to about 9%, about 5% to about 8%, about 5% to about 7%, about 5% to about 6%, about 6% to about 15%, about 6% to about 14%, about 6% to about 13%, about 6% to about 12%, about 6% to about 11%, about 6% to about 10%, about 6% to about 9%, about 6% to about 8%, about 6% to about 7%, about 7% to about 15%, about 7% to about 14%, about 7% to about 13%, about 7% to about 12%, about 7% to about 11%, about 7% to about 10%, about 7% to about 9%, about 7% to about 8%, about 8% to about 15%, about 8% to about 14%, about 8% to about 13%, about 8% to about 12%, about 8% to about 11%, about 8% to about 10%, about 8% to about 9%, about 9% to about 15%, about 9% to about 14%, about 9% to about 13%, about 9% to about 12%, about 9% to about 11%, about 9% to about 10%, about 10% to about 15%, about 10% to about 14%, about 10% to about 13%, about 10% to about 12%, about 10% to about 11%, about 11% to about 15%, about 11% to about 14%, about 11% to about 13%, about 11% to about 12%, about 12% to about 15%, about 12% to about 14%, about 12% to about 13%, about 13% to about 15%, about 13% to about 14%, or about 14% to about 15%) of PAX6+ cells.

In some embodiments, the cell population is identified as a suitable cell population for treatment of an eye disease when the cell population comprises at most about 10% or about 5% PAX6-positive cells. In certain embodiments, the cell population is identified as a suitable cell population for treatment of an eye disease when the cell population comprises at most about 10% PAX6-positive cells, at most about 0.05% Ki67-positive cells, at most about 30%, about 31%, about 32%, about 33%, about 34%, about 35%, about 36%, about 37%, about 38%, about 39%, about 40%, about 45%, about 50%, about 55%, about 60%, about 70%, about 80%, about 90%, about 95%, or about 100% CHX10-positive cells, and/or at most about 2% ONECUT1-positive cells. In some aspects, the cell population is identified as a suitable cell population for treatment of an eye disease when the cell population comprises at most about 5% PAX6-positive cells, at most about 0.04% Ki67-positive cells, at most about 15% CHX10-positive cells, and/or at most about 1% ON ECUT1-positive cells. In certain aspects, the cell population is identified as a suitable cell population for treatment of an eye disease when the cell population comprises at most about 5% PAX6-positive cells, at most about 0.04% Ki67-positive cells, at most about 15% CHX10-positive cells, and at most about 1% ONECUT1-positive cells.

In some embodiments, the cell population is identified as a suitable cell population (e.g., qualified for use as a therapeutic) for treatment of an eye disease when the cell population comprises at least about 90% of TUBB3 (e.g., about 90%, about 91%, about 92%, about 93%, about 94%, about 95%, about 96%, about 97%, about 98%, about 99%, or about 100% of TUBB3). In some embodiments, the cell population is identified as a suitable cell population (e.g., qualified for use as a therapeutic) for treatment of an eye disease when the cell population comprises at least about 90% to about 100% (e.g., about 90% to about 100%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 100%, about 91% to about 99%, about 91% to about 98%, about 91% to about 97%, about 91% to about 96%, about 91% to about 95%, about 91% to about 94%, about 91% to about 93%, about 91% to about 92%, about 92% to about 100%, about 92% to about 99%, about 92% to about 98%, about 92% to about 97%, about 92% to about 96%, about 92% to about 95%, about 92% to about 94%, about 92% to about 93%, about 93% to about 100%, about 93% to about 99%, about 93% to about 98%, about 93% to about 97%, about 93% to about 96%, about 93% to about 95%, about 93% to about 94%, about 94% to about 100%, about 94% to about 99%, about 94% to about 98%, about 94% to about 97%, about 94% to about 96%, about 94% to about 95%, about 95% to about 100%, about 95% to about 99%, about 95% to about 98%, about 95% to about 97%, about 95% to about 96%, about 96% to about 100%, about 96% to about 99%, about 96% to about 98%, about 96% to about 97%, about 97% to about 100%, about 97% to about 99%, about 97% to about 98%, about 98% to about 100%, about 98% to about 99%, or about 99% to about 100%) of Class Ill b-tubulin (TUBB3). In some embodiments, the cell population is identified as a suitable cell population for treatment of an eye disease when the cell population expresses one or more markers selected from the group consisting of: AIPL1, NR2E3, PPP4R4, OTX2, CRX, BLIMP 1, NEUROD1, RCVRN, TUBB3 and CD171/L1CAM.

In particular embodiments, the cell population is identified as a suitable cell population (e.g., qualified for use as a therapeutic) for treatment of an eye disease when the cell population comprises at least about 70% cell aggregate biomass (e.g., about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% cell aggregate biomass). In some embodiments, the cell population is identified as a suitable cell population (e.g., qualified for use as a therapeutic) for treatment of an eye disease when the cell population comprises between about 70% and about 100% (e.g., about 70% to about 100%, about 70% to about 90%, about 70% to about 80%, about 80% to about 100%, about 80% to about 90%, or about 90% to about 100%) cell aggregate biomass. In some embodiments, the cell population is identified as a suitable cell population for treatment of an eye disease when the cell population comprises at least about 70% cell aggregate biomass.

In some embodiments, the cell population is identified as a suitable cell population (e.g., qualified for use as a therapeutic) for treatment of an eye disease wherein the median diameter of a cell aggregate is about 15 μm to about 120 μm (e.g., about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, or about 120 μm). In some embodiments, the cell population is identified as a suitable cell population (e.g., qualified for use as a therapeutic) for treatment of an eye disease wherein the median diameter (e.g., particle diameter) of a cell aggregate is between about 15 μm and about 120 μm (e.g., about 15 μm to about 120 μm, about 20 μm to about 115 μm, about 35 μm to about 110 μm, about 35 μm to about 105 μm, about 40 μm to about 100 μm, about 45 μm to about 95 μm, about 50 μm to about 90 μm, about 55 μm to about 85 μm, about 60 μm to about 80 μm, about 65 μm to about 75 μm, about 30 μm to about 60 μm, about 40 μm to about 50 μm, about 20 μm to about 60 μm, about 20 μm to about 50 μm, or about 30 μm to about 60 μm). In some embodiments, the cell population is identified as a suitable cell population for treatment of an eye disease wherein the median diameter of a cell aggregate is between about 30 μm and about 60 μm. In some embodiments, the cell population is identified as a suitable cell population for treatment of an eye disease wherein the median diameter of a cell aggregate is about 50 μm. In some embodiments, the cell population is identified as a suitable cell population for treatment of an eye disease wherein the median diameter of a cell aggregate is at least about 20 μm.

In an aspect, provided herein is a method of identifying a suitable cell population for treatment of an eye disease, the method comprising: a) obtaining a population of cells comprising PRPs; b) measuring a percentage of PPP4R4+ cells in a cell population; and c) measuring a copy number of VIM per copy SDHA; wherein the cell population is identified as a suitable cell population for treatment of an eye disease when the percentage of PPP4R4+ cells in the cell population is between about 20% and about 60%, and the copy number of VIM per copy of SDHA is less than or equal to about 0.5 copies. In some embodiments, the method further comprises: d) measuring a percentage of AIPL1+ RCVRN+, PAX6+, CHX10+, TYRP1+; and/or Ki67+ cells in the cell population, wherein the cell population is identified as a suitable cell population for treatment of an eye disease when the percentage of AIPL1+ cells is least about 90%, the percentage of RCVRN+ cells is least about 90%, the percentage of PAX6+ cells is no more than about 7%, the percentage of CHX10+ cells is no more than about 7%, the percentage of TYRP1+ cells is no more than about 0.5%, and the percentage of Ki67+ cells is no more than about 0.5%.

In some embodiments, the cell population is identified as a suitable cell population (e.g., qualified for use as a therapeutic) for treatment of an eye disease where the cell population does not express LNCPRESS2 and/or AC009446.1 pluripotency markers above the limit of detection (LOD). Methods for detecting, characterizing, and/or quantitating, nucleic acid sequences; and for detecting, characterizing, and/or quantitating, mRNA expression, are known to persons skilled in the art, and include, but are not limited to, for example, PCR procedures, RT-PCR, quantitative PCR or RT-PCR, Northern blot analysis, differential gene expression, RNA protection assay, microarray analysis, hybridization methods, serial analysis of gene expression (SAGE), hybridization based on digital barcode quantification assays, multiplex RT-PCR, ddPCR, qRT-PCR, qPCR, UV spectroscopy, DNA sequencing, RNA sequencing, next-generation sequencing, including RNA-seq, lysate-based hybridization assays utilizing branched DNA signal amplification, such as the QuantiGene 2.0 Single Plex, and branched DNA analysis methods. In some embodiments, the cell population is identified as a suitable cell population for treatment of an eye disease where the cell population does not express LNCPRESS2 and/or AC009446.1 pluripotency markers above the limit of detection as measured by droplet digital polymerase chain reaction (ddPCR).

### Methods of Treating an Inherited Retinal Disease

The disclosure also provides a method of treating an inherited retinal disease (e.g., a primary photoreceptor disease such as retinitis pigmentosa, Usher Syndrome, cone-rod disease, rod-cone disease, cone dystrophy, and cone-rod dystrophy) in an individual in need thereof. Such methods may comprise administering a composition (e.g., a pharmaceutical composition as described herein) comprising photoreceptor precursor cells to said individual. Also provided is a composition (e.g., a pharmaceutical composition) as described herein for use as a medicament, or for use in a method of treating a retinal disease (e.g., a primary photoreceptor disease such as retinitis pigmentosa, Usher Syndrome, cone-rod disease, rod-cone disease, cone dystrophy, and cone-rod dystrophy). The composition may be administered surgically or as a surgical injection to the eye, and/or subretinal space of the eye. Said composition may be administered to the eye, subretinal space, or intravenously. Certain aspects provide a method to produce a composition comprising PRP cells or a PRP-enriched cell population which can be used for a number of important research, development, and commercial purposes.

The composition comprising the human PRP cells described herein, or a pharmaceutical composition including these cells, can be used for the manufacture of a medicament to treat a condition in a patient in need thereof. The composition comprising the PRP cells can be previously cryopreserved. In certain aspects, the disclosed composition comprising the PRP cells are derived from iPSCs, and thus can be used to provide “personalized medicine” for patients with eye diseases. In some embodiments, somatic cells obtained from patients can be genetically engineered to correct the disease-causing mutation, differentiated into PRP, and engineered to form a PRP tissue. This PRP tissue can be used to replace the endogenous degenerated PRP of the same patient.

Various eye conditions may be treated or prevented by the introduction of the composition comprising the PRP cells obtained using the methods disclosed herein. The conditions include retinal diseases or disorders generally associated with retinal dysfunction or degradation, retinal injury, and/or loss of retinal pigment epithelium. Conditions that can be treated include, without limitation, retinitis pigmentosa, Usher Syndrome, cone-rod disease, rod-cone disease, cone dystrophy, and cone-rod dystrophy.

In certain embodiments, methods are provided for treating or preventing a condition characterized by retinal degeneration, comprising administering to a subject in need thereof an effective amount of a composition comprising PRP cells (e.g., as described herein). The composition comprising the PRP cells may be transplanted in various formats. For example, the composition comprising the PRP cells may be introduced into the target site in the form of cell suspension, or adhered onto a matrix, extracellular matrix, or substrate such as a biodegradable polymer, as a monolayer, or a combination. In some embodiments, the composition comprising the PRP cells are produced from iPSCs from the subject to be treated, and thus are autologous. In other embodiments, the composition comprising the PRP cells are produced from a donor.

In certain embodiments, methods are provided for treating a disease or disorder characterized by retinal degeneration, comprising administering to a subject in need thereof an effective amount of a composition comprising PRP cells. These methods may include selecting a subject with such a disease or disorder and administering a therapeutically effective amount of the composition comprising PRP cells sufficient to treat the condition and/or ameliorate symptoms of the condition. The photoreceptor precursor cells may comprise at least about 50%, at least about 75%, at least about 85%, at least about 95%, at least about 99% or about 100% of the cells in the culture.

In one aspect, the cells can treat or alleviate the symptoms of retinitis pigmentosa in a patient in need of the treatment. The cells can be autologous or allogeneic to the patient. In a further aspect, the cells of the present disclosure can be administered in combination with other treatments.

In some embodiments, the composition comprising the PRP cells can be used for autologous PRP grafts to those subjects suitable for receiving regenerative medicine. The composition comprising the PRP cells may be transplanted in combination with other retinal cells, such as with photoreceptors. Transplantation of the composition comprising the PRP cells produced by the disclosed methods can be performed by various techniques known in the art. In accordance with one embodiment, the transplantation is performed via pars plana vitrectomy surgery followed by delivery of the cells through a small retinal opening into the sub-retinal space or by direct injection. The composition comprising the PRP cells can be introduced into the target site in the form of cell suspension, cell aggregates, adhered onto a matrix, such as extracellular matrix, or provided on substrate such as a biodegradable polymer. Thus, a composition comprising PRP cells obtained by the methods disclosed herein is provided.

In some embodiments, a composition (e.g., a pharmaceutical composition or a drug product as described herein) is provided that comprises at least about 104, about 10⁵, about 10⁶, or about 10⁷ cells (or any range derivable therein) comprising at least about 50% (for example, at least about 50%, about 60%, about 70%, about 80%, about 85%, about 90%, about 95%, about 96%, about 97%, about 98%, about 99%, about 99.5%, or any range derivable therein) PRP cells.

In some embodiments, a composition (e.g., an administered dose) is provided that comprises at least about 1×10⁶, 2×10⁶, 3×10⁶, 4×10⁶, or about 5×10⁶ cells (or any range derivable therein) comprising at least about 50% (for example, at least about 50%, about 60%, about 70%, about 80%, about 85%, about 90%, about 95%, about 96%, about 97%, about 98%, about 99%, about 99.5%, or any range derivable therein) PRP cells.

The composition comprising the PRP cells or photoreceptor cells produced by any suitable method may be used in any methods and applications currently known in the art for PRP or photoreceptor cells. The composition comprising the PRP cells or compositions of cells derived from the PRP cells may be used for transplantation such as cell rescue therapy or whole tissue replacement therapy. The cells of the present embodiments may also be used to produce retinal disease models to study pathophysiology and for drug screening.

The therapeutically effective amount of the composition described herein comprising photoreceptor precursor cells may be administered to a subject at a dose of about 0.5×10⁶ to about 5×10⁶ cells (e.g., viable cells) optionally with an immunosuppressive agent and/or antibiotic. For example, the therapeutically effective amount of the composition comprising photoreceptor precursor cells may be administered to a subject at a dose of about 0.5×10⁶ cells, about 0.75×10⁶ cells, about 1.0×10⁶ cells, about 1.25×10⁶ cells, about 1.5×10⁶ cells, about 1.75×10⁶ cells, about 2.0×10⁶ cells, about 2.25×10⁶ cells, about 2.5×10⁶ cells, about 2.75×10⁶ cells, about 3.0×10⁶ cells, about 3.25×10⁶ cells, about 3.5×10⁶ cells, about 3.75×10⁶ cells, about 4.0×10⁶ cells, about 4.25×10⁶ cells, about 4.5×10⁶ cells, about 4.75×10⁶ cells, or about 5.0×10⁶ cells. In some embodiments, the therapeutically effective amount of the composition comprising photoreceptor precursor cells may be administered to a subject at a dose of about 0.5×10⁶ cells to about 1.0×10⁶ cells. In some embodiments, the therapeutically effective amount of the composition comprising photoreceptor precursor cells may be administered to a subject at a dose of about 1.0×10⁶ cells to about 1.5×10⁶ cells. In some embodiments, the therapeutically effective amount of the composition comprising photoreceptor precursor cells may be administered to a subject at a dose of about 1.5×10⁶ cells to about 2.0×10⁶ cells. In some embodiments, the therapeutically effective amount of the composition comprising photoreceptor precursor cells may be administered to a subject at a dose of about 2.0×10⁶ cells to about 2.5×10⁶ cells. In some embodiments, the therapeutically effective amount of the composition comprising photoreceptor precursor cells may be administered to a subject at a dose of about 2.5×10⁶ cells to about 3.0×10⁶ cells. In some embodiments, the therapeutically effective amount of the composition comprising photoreceptor precursor cells may be administered to a subject at a dose of about 3.0×10⁶ cells to about 3.5×10⁶ cells. In some embodiments, the therapeutically effective amount of the composition comprising photoreceptor precursor cells may be administered to a subject at a dose of about 3.5×10⁶ cells to about 4.0×10⁶ cells. In some embodiments, the therapeutically effective amount of the composition comprising photoreceptor precursor cells may be administered to a subject at a dose of about 4.0×10⁶ cells to about 4.5×10⁶ cells. In some embodiments, the therapeutically effective amount of the composition comprising photoreceptor precursor cells may be administered to a subject at a dose of about 4.5×10⁶ cells to about 5.0×10⁶ cells. In some embodiments, the therapeutically effective amount of the composition comprising photoreceptor precursor cells may be administered to a subject at a dose of about 0.5×10⁶ cells, about 0.75×10⁶ cells, about 1.0×10⁶ cells, about 1.25×10⁶ cells, about 1.5×10⁶ cells, about 1.75×10⁶ cells, about 2.0×10⁶ cells, about 2.25×10⁶ cells, about 2.5×10⁶ cells, about 2.75×10⁶ cells, about 3.0×10⁶ cells, about 3.25×10⁶ cells, about 3.5×10⁶ cells, about 3.75×10⁶ cells, about 4.0×10⁶ cells, about 4.25×10⁶ cells, about 4.5×10⁶ cells, about 4.75×10⁶ cells, or about 5.0×10⁶ cells. In some embodiments, the therapeutically effective amount of the composition comprising photoreceptor precursor cells may be administered to a subject at a dose of about 1×10⁶ to about 4×10⁶ cells (e.g., viable cells) optionally with an immunosuppressive agent and/or antibiotic.

In some embodiments, the therapeutically effective amount of the composition comprising photoreceptor precursor cells may be administered to a subject at a dose of at least about 0.5×10⁶ to at least about 5×10⁶ cells. For example, the therapeutically effective amount of the composition comprising photoreceptor precursor cells may be administered to a subject at a dose of at least about 0.5×10⁶ cells, about 0.75×10⁶ cells about 1.0×10⁶ cells, about 1.25×10⁶ cells, about 1.5×10⁶ cells, about 1.75×10⁶ cells, about 2.0×10⁶ cells, about 2.25×10⁶ cells, about 2.5×10⁶ cells, about 2.75×10⁶ cells, about 3×10⁶ cells, about 3.25×10⁶ cells, about 3.5×10⁶ cells, about 3.75×10⁶ cells, about 4.0×10⁶ cells, about 4.25×10⁶ cells, at least 4.5×10⁶ cells, about 4.75×10⁶ cells, or about 5.0×10⁶ cells. In some embodiments, the therapeutically effective amount of the composition comprising photoreceptor precursor cells may be administered to a subject at a dose of at least about 0.5×10⁶ cells to at least about 1.0×10⁶ cells. In some embodiments, the therapeutically effective amount of the composition comprising photoreceptor precursor cells may be administered to a subject at a dose of at least about 1.0×10⁶ cells to at least about 1.5×10⁶ cells. In some embodiments, the therapeutically effective amount of the composition comprising photoreceptor precursor cells may be administered to a subject at a dose of at least about 1.5×10⁶ cells to at least about 2.0×10⁶ cells. In some embodiments, the therapeutically effective amount of the composition comprising photoreceptor precursor cells may be administered to a subject at a dose of at least about 2.0×10⁶ cells to at least about 2.5×10⁶ cells. In some embodiments, the therapeutically effective amount of the composition comprising photoreceptor precursor cells may be administered to a subject at a dose of at least about 2.5×10⁶ cells to at least about 3.0×10⁶ cells. In some embodiments, the therapeutically effective amount of the composition comprising photoreceptor precursor cells may be administered to a subject at a dose of at least about 3.0×10⁶ cells to at least about 3.5×10⁶ cells. In some embodiments, the therapeutically effective amount of the composition comprising photoreceptor precursor cells may be administered to a subject at a dose of at least about 3.5×10⁶ cells to at least about 4.0×10⁶ cells. In some embodiments, the therapeutically effective amount of the composition comprising photoreceptor precursor cells may be administered to a subject at a dose of at least about 4.0×10⁶ cells to at least about 4.5×10⁶ cells. In some embodiments, the therapeutically effective amount of the composition comprising photoreceptor precursor cells may be administered to a subject at a dose of at least about 4.5×10⁶ cells to at least about 5.0×10⁶ cells. In some embodiments, the therapeutically effective amount of the composition comprising photoreceptor precursor cells may be administered to a subject at a dose of at least about 1.0×10⁶ to at least about 4.0×10⁶ cells.

In some embodiments, the therapeutically effective amount of the composition comprising photoreceptor precursor cells is administered at a single time to the subject. In some embodiments, the therapeutically effective amount of the composition comprising photoreceptor precursor cells is administered at multiple times (e.g., 2, 3, 4, 5, 6, 7, 8, 9, 10 or more times) to the subject.

In some embodiments, the therapeutically effective amount of the composition comprising photoreceptor precursor cells is surgically administered to a target site in the subject. In some embodiments, the therapeutically effective amount of the composition comprising photoreceptor precursor cells may be surgically administered as a single dose. In some embodiments, the therapeutically effective amount of the composition comprising photoreceptor precursor cells may be surgically administered in multiple doses. The composition comprising PRP cells may be introduced into the target site in the form of cell suspension, or adhered onto a matrix, extracellular matrix, or substrate such as a biodegradable polymer, as a monolayer, or a combination. The composition comprising PRP cells may be introduced into the target site in the form of a cell suspension comprising cell aggregates.

In some embodiments, the composition comprising PRP cells is formulated as a pharmaceutical composition or a drug product. In some embodiments, the pharmaceutical composition or the drug product comprises the composition of PRP cells and CryoSTOR® cell cryopreservation media (e.g., CS10).

In some embodiments, the composition comprising PRP cells is formulated as a pharmaceutical composition or a dose for administration. In some embodiments, the pharmaceutical composition or the dose for administration comprises the composition of PRP cells, a Balanced Salt Solution (BSS™ Sterile Irrigating Solution; Alcon Laboratories, Inc. Cat. No. 65079550), and/or Human Serum Albumin (HSA; Octapharma Cat. No. 68982-0643-02). In some embodiments, the BSS comprises magnesium chloride, calcium chloride, potassium chloride, sodium citrate, sodium acetate, and/or sodium chloride.

In some embodiments, the balanced salt solution comprises about 0.0 to 1.50 mg/mL of magnesium chloride, about 0.0 mg/mL to about 1.5 mg/mL of calcium chloride, about 0.0 mg/mL to about 1.5 mg/mL of potassium chloride, about 0.0 mg/mL to about 3.0 mg/mL of sodium citrate, about 0.0 mg/mL to about 6.0 mg/mL of sodium acetate, and/or about 0.0 mg/mL to about 10.0 mg/mL of sodium chloride. In some embodiments, the balanced salt solution comprises about 0.0 mg/mL, about 0.10 mg/mL, about 0.20 mg/mL, about 0.30 mg/mL, about 0.40 mg/mL, about 0.50 mg/mL, about 0.60 mg/mL, about 0.70 mg/mL, about 0.80 mg/mL, about 0.90 mg/mL, about 1.00 mg/mL, about 1.10 mg/mL, about 1.20 mg/mL, about 1.30 mg/mL, about 1.40 mg/mL, or about 1.50 mg/mL of magnesium chloride. In some embodiments, the balanced salt solution comprises about 0.0 mg/mL, about 0.10 mg/mL, about 0.20 mg/mL, about 0.30 mg/mL, about 0.40 mg/mL, about 0.50 mg/mL, about 0.60 mg/mL, about 0.70 mg/mL, about 0.80 mg/mL, about 0.90 mg/mL, about 1.00 mg/mL, about 1.10 mg/mL, about 1.20 mg/mL, about 1.30 mg/mL, about 1.40 mg/mL, or about 1.50 mg/mL of calcium chloride. In some embodiments, the balanced salt solution comprises about 0.0 mg/mL, about 0.10 mg/mL, about 0.20 mg/mL, about 0.30 mg/mL, about 0.40 mg/mL, about 0.50 mg/mL, about 0.60 mg/mL, about 0.70 mg/mL, about 0.80 mg/mL, about 0.90 mg/mL, about 1.00 mg/mL, about 1.10 mg/mL, about 1.20 mg/mL, about 1.30 mg/mL, about 1.40 mg/mL, or about 1.50 mg/mL of potassium chloride. In some embodiments, the balanced salt solution comprises about 0.0 mg/mL, about 0.10 mg/mL, about 0.20 mg/mL, about 0.30 mg/mL, about 0.40 mg/mL, about 0.50 mg/mL, about 0.60 mg/mL, about 0.70 mg/mL, about 0.80 mg/mL, about 0.90 mg/mL, about 1.00 mg/mL, about 1.10 mg/mL, about 1.20 mg/mL, about 1.30 mg/mL, about 1.40 mg/mL, 1.50 mg/mL, about 1.60 mg/mL, about 1.70 mg/mL, about 1.80 mg/mL, about 1.90 mg/mL, about 2.00 mg/mL, about 2.10 mg/mL, about 2.20 mg/mL, about 2.30 mg/mL, about 2.40 mg/mL, about 2.50 mg/mL, about 2.60 mg/mL, about 2.70 mg/mL, about 2.80 mg/mL, about 2.90 mg/mL, or about 3.00 mg/mL of sodium citrate. In some embodiments, the balanced salt solution comprises about 0.0 mg/mL, about 0.25 mg/mL, about 0.50 mg/mL, about 0.75 mg/mL, about 1.00 mg/mL, about 1.25 mg/mL, about 1.50 mg/mL, about 1.75 mg/mL, about 2.00 mg/mL, about 2.25 mg/mL, about 2.50 mg/mL, about 2.75 mg/mL, about 3.00 mg/mL, about 3.25 mg/mL, about 3.50 mg/mL, 3.75 mg/mL, about 4.00 mg/mL, about 4.25 mg/mL, about 4.50 mg/mL, about 4.75 mg/mL, about 5.00 mg/mL, about 5.25 mg/mL, about 5.50 mg/mL, about 5.75 mg/mL, or about 6.00 mg/mL of sodium acetate. In some embodiments, the balanced salt solution comprises about 0.0 mg/mL, about 0.50 mg/mL, about 1.00 mg/mL, about 1.50 mg/mL, about 2.50 mg/mL, about 3.00 mg/mL, about 3.50 mg/mL, about 4.00 mg/mL, about 4.50 mg/mL, about 5.00 mg/mL, about 5.50 mg/mL, about 6.00 mg/mL, about 6.50 mg/mL, about 7.00 mg/mL, 7.50 mg/mL, about 8.00 mg/mL, about 8.50 mg/mL, about 9.00 mg/mL, about 9.50 mg/mL, or about 10.00 mg/mL of sodium chloride.

In some embodiments, the human serum albumin is at a concentration of about 0.0% to about 1.0% (e.g., about 0.0% to about 1.0%, about 0.0% to about 0.9%, about 0.0% to about 0.8%, about 0.0% to about 0.7%, about 0.0% to about 0.6%, about 0.0% to about 0.5%, about 0.0% to about 0.4%, about 0.0% to about 0.3%, about 0.0% to about 0.2%, about 0.0% to about 0.1%, about 0.1% to about 1.0%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.0%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.0%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.0%, about 0.4% to about 0.9%, about 0.4% to about 0.8%, about 0.4% to about 0.7%, about 0.4% to about 0.6%, about 0.4% to about 0.5%, about 0.5% to about 1.0%, about 0.5% to about 0.9%, about 0.5% to about 0.8%, about 0.5% to about 0.7%, about 0.5% to about 0.6%, about 0.6% to about 1.0%, about 0.6% to about 0.9%, about 0.6% to about 0.8%, about 0.6% to about 0.7%, about 0.7% to about 1.0%, about 0.7% to about 0.9%, about 0.7% to about 0.8%, about 0.8% to about 1.0%, about 0.8% to about 0.9%, or about 0.9% to about 1.0%). In some embodiments, the human serum albumin is at a concentration of about 0.0%, about 0.1%, about 0.2%, about 0.3%, about 0.4%, about 0.5%, about 0.6%, about 0.7%, about 0.8%, about 0.9%, or about 1.0%. In some embodiments, the human serum albumin is at a concentration of about 0.2%.

In some embodiments, the balanced salt solution comprises about 0.3 mg/mL of magnesium chloride, about 0.48 mg/mL of calcium chloride, about 0.75 mg/mL of potassium chloride, about 1.7 mg/mL of sodium citrate, about 3.9 mg/mL of sodium acetate, and/or about 6.4 mg/mL of sodium chloride. In some embodiments, the human serum albumin is at a concentration of about 0.2%.

In one aspect, the composition comprising PRP cells may treat or alleviate the symptoms of retinitis pigmentosa, cone-rod disease, rod-cone disease, cone dystrophies, cone-rod and/or rod-cone dystrophies, or Usher Syndrome. In a further aspect, the composition comprising PRPs may be administered in combination with other treatments including standard of care therapies.

The disclosure also provides methods of treating vision loss in a subject in need thereof, the methods comprising administering a therapeutically effective amount of one or more immunosuppressive agents to the subject. Such methods may further comprise administering a therapeutically effective amount of the composition comprising photoreceptor precursor cells into a subretinal space of an eye of the subject.

Additionally, the disclosure also provides methods of increasing a thickness of an outer nuclear layer of a retina of a human subject. Such methods may further comprise surgically administering a composition comprising a therapeutically effective amount of photoreceptor precursor cells to a subretinal space of an eye of the human subject.

In some embodiments, the composition comprising PRPs may be administered via surgery using commercial, off-the-shelf ophthalmic and retinal surgical instruments. In some embodiments, the composition comprising PRP cells may be administered in a surgical suite under controlled aseptic conditions in a single surgical session while the subject is under general anesthesia.

In some embodiments, the composition comprising PRPs may be loaded into a delivery device, such as a CONSTELLATION® Vision System (Alcon Science #8065753048, Geneva, Switzerland). Basic procedure components for operating the Constellation system may be provided in the VFC Pak, including tubing (Alcon #8065750957). A syringe (e.g., a MicroDose Injection Kit (MedOne #3275)) and cannula (e.g., Sterilized PolyTip Funnel Cannula 25/33g (MedOne #5002)) may also be used. In some embodiments, methods for use of the syringe and/or cannula is described in WO 2024/073759 A1, which is incorporated herein by reference in its entirety.

In some embodiments, the Constellation system may be primed using a vehicle solution (BSS™ Sterile Irrigating Solution [balanced salt solution](Alcon Laboratories, Inc., Geneva, Switzerland) supplemented with about 0.02% to about 0.40% human serum albumin), followed by the withdrawal of vehicle solution (e.g., the majority of vehicle solution). In some embodiments, the vehicle solution of BSS™ Sterile Irrigating Solution may be supplemented with about 0.02%, about 0.04%, about 0.06%, about 0.08%, about 0.10%, about 0.12%, about 0.14%, about 0.16%, about 0.18%, about 0.20%, about 0.22%, about 0.24%, about 0.26%, about 0.28%, about 0.30%, about 0.32%, about 0.34%, about 0.36%, about 0.38%, or about 0.40% human serum albumin. In some embodiments, the vehicle solution of BSS™ Sterile Irrigating Solution may be supplemented with about 0.20% human serum albumin.

The pharmaceutical compositions can be optionally packaged in a suitable container with written instructions for a desired purpose, such as the reconstitution of PRP cell function to improve a disease or abnormality of the retinal tissue. In some embodiments, the composition comprising the PRP cells produced by the disclosed methods may be used to replace degenerated photoreceptor cells of a subject in need therein.

In some embodiments, a pharmaceutical composition comprising PRP cells may exhibit an endotoxin level of less than or equal to about 5.00 EU/mL as determined by Nexgen Endosafe Systems (Charles River Laboratories, Wilmington, MA). In some embodiments, the pharmaceutical composition comprising PRP cells may exhibit an endotoxin level of about 0.00 EU/mL, about 0.25 EU/mL, about 0.50 EU/mL, about 0.75 EU/mL, about 1.00 EU/mL, about 1.25 EU/mL, about 1.50 EU/mL, about 1.75 EU/mL, about 2.00 EU/mL, about 2.25 EU/mL, about 2.50 EU/mL, about 2.75 EU/mL, about 3.00 EU/mL, about 3.25 EU/mL, about 3.50 EU/mL, about 3.75 EU/mL, about 4.00 EU/mL, about 4.25 EU/mL, about 4.50 EU/mL, about 4.75 EU/mL, or about 5.00 EU/mL as determined by Nexgen Endosafe Systems. In some embodiments the pharmaceutical composition comprising PRP cells exhibit an endotoxin level of at most about 0.025 EU/mL.

In some embodiments, a pharmaceutical composition comprising PRP cells may appear as white or off white settled cells upon visual inspection. In some embodiments, the pharmaceutical composition comprising PRP cells may appear as a homogenous cell suspension upon visual inspection. In some embodiments, the pharmaceutical composition comprising PRP cells may appear as a translucent, colorless fluid. In other embodiments, the pharmaceutical composition comprising PRP cells may appear as an opaque, colorless fluid. In some embodiments, the pharmaceutical composition comprising the PRP cells may appear essentially free of visible particulates and foreign matter. In some embodiments, the pharmaceutical composition comprising PRP cells may appear as white or off-white settled cells or homologous cell suspension, in a translucent or opaque colorless fluid, essentially free of visible particulates and foreign matter.

In some embodiments, the live cell concentration of a pharmaceutical composition comprising PRP cells may be calculated using automated cell counting with acridine orange and/or propidium iodide using, for example, a Cellaca™ MX High Throughput Cell Counter (Revvity, Waltham, MA, Part No.: MX-AOPI). In some embodiments, the live cell concentration of the pharmaceutical composition comprising PRP cells for a dose comprising about 1×10⁶ PRP cells per 50 μL may have a concentration of about 2.72×10⁷ cells/mL to about 4.08×10⁷ cells/mL as determined by automated cell counting. In some embodiments, the live cell concentration of the pharmaceutical composition comprising PRP cells for a dose comprising about 2×10⁶ PRP cells per 50 μL may have a concentration of about 5.04×10⁷ cells/mL to about 7.56×10⁷ cells/mL as determined by automated cell counting. In some embodiments, the live cell concentration of the pharmaceutical composition comprising PRP cells for a dose comprising about 3×10⁶ PRP cells per 50 μL may have a concentration of about 7.12×10⁷ cells/mL to about 1.07×10⁸ cells/mL as determined by automated cell counting. In some embodiments, the live cell concentration of the pharmaceutical composition comprising PRP cells for a dose comprising about 4×10⁶ PRP cells per 50 μL may have a concentration of about 9.44×10⁷ cells/mL to about 1.42×10⁸ cells/mL as determined by automated cell counting.

In some embodiments, a percentage cell viability of a pharmaceutical composition comprising PRP cells may be calculated using automated cell counting with acridine orange and/or propidium iodide using, for example, a Cellaca™ MX. In some embodiments, the percentage viability of the pharmaceutical composition comprising PRP cells may be calculated to be between about 30% and about 110%. In some embodiments, the percentage viability of the pharmaceutical composition comprising PRP cells may be calculated to be about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, or about 110%. In some embodiments, the percentage viability of the pharmaceutical composition comprising PRP cells may be calculated to be at least about 40%.

In some embodiments, the sterility of the pharmaceutical composition comprising PRP cells may be assessed for contaminant growth. In some embodiments, the pharmaceutical composition comprising PRP cells may exhibit no contaminant growth.

Methods of Making Photoreceptor Precursor Cells from Induced Pluripotent Stem Cells (iPSCs)

Somatic cells can be reprogrammed to produce induced pluripotent stem cells (iPSCs) using methods known to one of skill in the art see, e.g., Published U.S. Patent Application No. 2009/0246875, Published U.S. Patent Application No. 2010/0210014; Published U.S. Patent Application No. 20120276636; U.S. Pat. Nos. 8,058,065; 8,129,187; 8,278,620; PCT Publication NO. WO 2007/069666 A1, and U.S. Pat. No. 8,268,620, which are incorporated herein by reference.

With the exception of germ cells, any somatic cell can be used as a starting point to make an iPSC. For example, cell types could be keratinocytes, fibroblasts, hematopoietic cells, mesenchymal cells, liver cells, or stomach cells. T cells may also be used as a source of somatic cells for reprogramming. There is no limitation on the degree of cell differentiation or the age of an animal from which cells are collected; even undifferentiated progenitor cells (including somatic stem cells) and finally differentiated mature cells can be used as sources of somatic cells in the methods disclosed herein. The somatic cell itself may be a PRP cell, such as a human PRP cell. The PRP cell can be an adult or a fetal PRP cell. iPSCs can be grown under conditions that are known to differentiate human embryonic stem cells into specific cell types, and express human embryonic stem cell markers including, for example, SSEA-1, SSEA-3, SSEA-4, TRA-1-60, and/or TRA-1-81.

Generally, nuclear reprogramming factors may be used to produce pluripotent stem cells from a somatic cell. For example, cells may be treated with a nuclear reprogramming substance, which may be generally one or more factor(s) capable of inducing an iPSC from a somatic cell or a nucleic acid that encodes these substances. The nuclear reprogramming substances generally include at least Oct3/4, Klf4 and Sox2 or nucleic acids that encode these molecules. A functional inhibitor of p53, L-myc or a nucleic acid that encodes L-myc, and Lin28, or Lin28b or a nucleic acid that encodes Lin28 or Lin28b, can be utilized as additional nuclear reprogramming substances. Nanog can also be utilized for nuclear reprogramming. In some embodiments, at least two, at least three, or at least four of Klf4, c-Myc, Oct3/4, Sox2, Nanog, and Lin28 are utilized. In other embodiments, Oct3/4, Sox2, c-Myc and Klf4 are utilized.

As disclosed in U.S. Pat. No. 8,900,871, exemplary reprogramming factors for the production of iPSCs include (1) Oct3/4, Klf4, Sox2, L-Myc (Sox2 can be replaced with SoxI, Sox3, Sox15, Sox17 or Sox18; Klf4 may be replaceable with KlfI, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)I6 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, BmiI; (7) Oct3/4, Klf4, Sox2, L-Myc, Lin28; (8) Oct3/4, Klf4, Sox2, L-Myc, Lin28, SV 40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb may be replaceable with Esrrg); (12) Oct3/4, Klf4, Sox2; (13) Oct3/4, Klf4, Sox2, TERT, SV 40LT; (14) Oct3/4, Klf4, Sox2, TERT, HP VI 6 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, BmiI; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb may be replaceable with Esrrg). Factors like Nanog, Lin28, Klf4, or c-Myc can increase reprogramming efficiency and can be expressed from several different expression vectors. For example, an integrating vector such as the EBV element-based system can be used (U.S. Pat. No. 8,546,140). Reprogramming proteins could be introduced directly into somatic cells by protein transduction. Reprogramming may further comprise contacting the cells with one or more signaling receptors including glycogen synthase kinase 3 (GSK-3) inhibitor, a mitogen-activated protein kinase kinase (MEK) inhibitor, a transforming growth factor beta (TGF-P) receptor inhibitor or signaling inhibitor, leukemia inhibitory factor (LIF), a p53 inhibitor, an NF-kappa B inhibitor, or a combination thereof. Those regulators may include small molecules, inhibitory nucleotides, expression cassettes, or protein factors.

Mouse and human cDNA sequences of these nuclear reprogramming substances are available with reference to the NCBI accession numbers mentioned in WO 2007/069666, which is incorporated herein by reference. Methods for introducing one or more reprogramming substances, or nucleic acids encoding these reprogramming substances, are known in the art, and disclosed for example, in U.S. Pat. Nos. 8,900,871 and 8,071,369, which both are incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Publication No. 2003/0211603. Other methods for the culture and maintenance of iPSCs, as would be known to one of skill in the art, may be used.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium (Ludwig et. al., 2006a; Ludwig et. al., 2006b) or E8™ medium (Chen et al., 2011).

In some embodiments, the iPSC can be modified to express exogenous nucleic acids, such as to include an enhancer operably linked to a promoter and a nucleic acid sequence encoding a first marker. Suitable promoters include, but are not limited to, any promoter expressed in photoreceptor cells, such as a rhodopsin kinase promoter. The construct can also include other elements, such as a ribosome binding site for translational initiation (internal ribosomal binding sequences), and/or a transcription/translation terminator. Generally, it is advantageous to transfect cells with the construct. Suitable vectors for stable transfection include, but are not limited to retroviral vectors, lentiviral vectors, and Sendai virus.

In some embodiments, plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer, and (4) a multicloning site for incorporation of various nucleic acid cassettes; and (5) a nucleic acid sequence encoding a marker operably linked to the tyrosinase promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, which are incorporated herein by reference.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV 40) based episomal vector, a bovine papilloma virus (BPV)-based vector, or a lentiviral vector.

Markers include, but are not limited to, fluorescence proteins (for example, green fluorescent protein or red fluorescent protein), enzymes (for example, horse radish peroxidase or alkaline phosphatase or firefly/renilla luciferase or nanoluc), or other proteins. A marker may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe, or nucleic acid amplification reaction that are specific for the marker of the cell type of interest. The markers can also be identified by a biochemical or enzyme assay or biological response that depends on the function of the gene product. Nucleic acid sequences encoding these markers can be operably linked to the tyrosinase enhancer. In addition, other genes can be included, such as genes that may influence stem cell to PRP differentiation, or photoreceptor function, or physiology, or pathology.

In some embodiments, neural retinal progenitor (NRP) cells may be used in the methods disclosed herein. In some embodiments, photoreceptor precursor (PRP) cells may be used in the methods disclosed herein. Such cells may be obtained from iPSCs. In some embodiments, the composition comprising the PRPs obtained from an iPSC may be termed “induced” PRPs (iPRPs). PRP cells may express one or more markers such as AIPL1, OTX2, CRX, NR2E3, PPP4R4, PRDM1 (BLIMP1), NEUROD1, RCVRN, TUBB3 and LICAM (CD171). PRP cells may express several proteins that can serve as markers for detection by the use of methodologies, such as immunocytochemistry, Western blot analysis, flow cytometry, or enzyme-linked immunoassay (ELISA). For example, one characteristic PRP-marker is RCVRN. PRP cells may not express (at any detectable level) the embryonic stem cells markers OCT-4, NANOG, or REX-1. Specifically, expression of these genes may be approximately 100 to 1,000-fold lower in PRP cells than in iPSC cells when assessed by quantitative RT-PCR.

PRP cell markers may be detected at the mRNA level, for example, by reverse transcriptase polymerase chain reaction (RT-PCR), Northern blot analysis, or dot-blot hybridization analysis using sequence-specific primers in standard amplification methods using publicly available sequence data (GENBANK®). Expression of tissue-specific markers as detected at the protein or mRNA level may be considered positive if the level is at least about 2-, at least about 3-, at least about 4-, at least about 5-, at least about 6-, at least about 7-, at least about 8-, or at least about 9-fold, and more particularly more than at least about 10, at least about 20-, at least about 30-, at least about 40-, at least about 50-fold or higher above that of a control cell, such as an undifferentiated pluripotent stem cell or other unrelated cell type.

In some embodiments, methods are provided for producing a composition comprising PRP cells from an essentially single cell suspension of PSCs such as human iPSCs. In some embodiments, the PSCs are cultured to pre-confluency to prevent any cell aggregates. In certain aspects, the PSCs are dissociated by incubation with a cell dissociation solution or enzyme, such as by Versene (ThermoFisher Scientific, Waltham, MA; Cat. No.: 15040066), trypsin, ACCUTASE™ (STEMCELL Technologies, Vancouver, Canada Cat. No.: 07920) or TrypLE™ (ThermoFisher, Waltham, MA; Cat. No.: 12604013). PSCs can also be dissociated into an essentially single cell suspension by pipetting.

In addition, blebbistatin (e.g., about 2.5 μM) can be added to the medium to increase PSC survival after dissociation into single cells while the cells are not adhered to a culture vessel. A rho kinase (ROCK) inhibitor instead of blebbistatin may alternatively be used to increase PSC survival after dissociation into single cells.

To efficiently differentiate PRP cells from the single cell PSCs, an accurate count of the input density can increase PRP differentiation efficiency. Thus, the single cell suspension of PSCs is generally counted before seeding. For example, the single cell suspension of PSCs is counted by a hemocytometer or an automated cell counter, such as a VI-CELL®. The cells may be diluted to a cell density of about 10,000 to about 500,000 cells/mL, about 50,000 to about 200,000 cells/mL, or about 75,000 to about 150,000 cells/mL. In a non-limiting example, the single cell suspension of PSCs is diluted to a density of about 100,000 cells/mL in a fully defined cultured medium such as ESSENTIAL 8™ (E8™) medium.

Once a single cell suspension of PSCs is obtained at a known cell density, the cells are generally seeded in an appropriate culture vessel, such as a tissue culture plate, such as a flask, 6-well, 24-well, or 96-well plate. A culture vessel used for culturing the cell(s) can include, but is particularly not limited to: flask, flask for tissue culture, dish, Petri dish, dish for tissue culture, multi dish, micro plate, micro-well plate, multi plate, multi-well plate, micro slide, chamber slide, tube, tray, CELLSTACK® Chambers (e.g., Corning®, Corning, NY; Cat., No: 3268), culture bag, and roller bottle, as long as it is capable of culturing the stem cells therein. The cells may be cultured in a volume of at least about 0.2 mL, at least about 0.5 mL, at least about 1 mL, at least about 2 mL, at least about 5 mL, at least about 10 mL, at least about 20 mL, at least about 30 mL, at least about 40 mL, at least about 50 mL, at least about 100 mL, at least about 150 mL, at least about 200 mL, at least about 250 mL, at least about 300 mL, at least about 350 mL, at least about 400 at least about mL, at least about 450 mL, at least about 500 mL, at least about 550 mL, at least about 600 mL, at least about 800 mL, at least about 1000 mL, at least about 1500 mL, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least about 2 L, at least about 4 L, at least about 5 L, at least about 6 L, at least about 8 L, at least about 10 L, at least about 15 L, at least about 20 L, at least about 25 L, at least about 50 L, at least about 75 L, at least about 100 L, at least about 150 L, at least about 200 L, at least about 500 liters, or at least about 1, at least about 2, at least about 4, at least about 6, at least about 8, at least about 10, at least about 15 cubic meters, or any range derivable therein.

In certain aspects, the PSCs, such as iPSCs, are plated at a cell density appropriate for efficient differentiation. Generally, the cells are plated at a cell density of about 1,000 to about 75,000 cells/cm², such as of about 5,000 to about 40,000 cells/cm². In a 6 well plate, the cells may be seeded at a cell density of about 50,000 to about 400,000 cells per well. In exemplary methods, the cells are seeded at a cell density of about 100,000, about 150,000, about 200,000, about 250,000, about 300,000 or about 350,000 cells per well, such as about 200,000 cells per well. In some embodiments, the cells may be provided as a suspension, a solution, or a pellet. In some embodiments, the composition comprising the PRPs are in a single cell form or an aggregate form (e.g., centrifuged) or a suspended (e.g., dispersed in solution) form.

The PSCs, such as iPSCs, are generally cultured on culture plates coated by one or more cellular adhesion proteins to promote cellular adhesion while maintaining cell viability. For example, preferred cellular adhesion proteins include extracellular matrix proteins such as vitronectin, laminin, collagen, and/or fibronectin, which may be used to coat a culturing surface as a means of providing a solid support for pluripotent cell growth. The term “extracellular matrix” or “ECM” is recognized in the art. Its components can include, but are not limited to, one or more of the following proteins: fibronectin, laminin, vitronectin, tenascin, entactin, thrombospondin, elastin, gelatin, collagen, fibrillin, merosm, anchorin, chondronectin, link protein, bone sialoprotein, osteocalcin, osteopontin, epinectin, hyaluronectin, undulin, epiligrin, and kalinin. Other ECM components may include synthetic peptides for adhesion (e.g., RGD or IKV AV motifs), synthetic hydrogels (e.g., PEG, PLGA, etc.) or natural hydrogels, such as alginate. In exemplary methods, the PSCs are grown on culture plates coated with vitronectin. In some embodiments, the cellular adhesion proteins are human proteins.

The extracellular matrix proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition is xeno-free. For example, in the xeno-free matrix to culture human cells, matrix components of human origin may be used, wherein any non-human animal components may be excluded.

In some aspects, the total protein concentration m the matrix composition may be about 1 ng/mL to about 1 mg/mL. In some preferred embodiments, the total protein concentration in the matrix composition is about 1 μg/mL to about 300 μg/mL. In more preferred embodiments, the total protein concentration in the matrix composition is about 5 μg/mL to about 200 μg/mL.

In some embodiments, the differentiation from iPSCs into photoreceptor precursor cells is performed as described in WO 2019/204817 A1, which is herein incorporated by reference in its entirety.

In some embodiments, the iPSCs are obtained from a working cell bank (WCB). In some embodiments, the WCB cells are thawed, expanded, and differentiated into induced photoreceptor precursor cells (iPRPs). In some embodiments, the iPRPs are formulated into cryopreservation medium (CryoSTOR® CS10 Cell Freezing Medium) and stored in the vapor phase of liquid nitrogen (about ≤−150° C.) as cryopreserved Drug Product (DP). In some embodiments, the cryopreserved DP undergoes a thaw, wash, and final formulation as a dose for administration (DfA) with Balanced Salt Solution (BSS®) Sterile Irrigating Solution supplemented with 0.2% Human Serum Albumin (HSA).

In some embodiments, the cryopreserved WCB iPSCs are thawed and seeded onto vitronectin-coated plates and cultured overnight. In some embodiments, the iPSCs are further cultured for about 1, about 2, about 3, about 4, about 5, or about 6 days with daily medium exchange. In some embodiments, the iPSCs are cultured for about three to about four days with daily medium exchange. In some embodiments, lactate concentration is assessed to monitor cell growth.

In some embodiments, the cells are passaged with ethylenediaminetetraacetic acid (EDTA), harvested, and seeded into vitronectin-coated flasks. In some embodiments, the iPSCs are cultured for an additional three days with daily medium exchange. In some embodiments, the cells are then passaged using TrypLE™ for aggregate dissociation. In some embodiments, the cells are then pooled, and the single cell suspension is analyzed for viable cell concentration. In some embodiments, the cells are then pooled and seeded into vitronectin-coated vessels and cultured for about two days with daily medium exchange.

In some embodiments, the cells are then cultured in vitronectin-coated vessels for about 6, about 7, about 8, about 9, about 10, about 11, about 12, about 13, about 14, about 15, about 16, or about 17 days with daily medium exchange. In some embodiments, the cells are cultured in vitronectin-coated vessels for about 11 days with daily medium exchange. In some embodiments, the cells are cultured in vitronectin-coated vessels for about 15 days with daily medium exchange.

In some embodiments, cells are then harvested with TrypLE™, centrifuged, resuspended, pooled, and strained to remove large clusters and isolate single cells. In some embodiments, the resulting single cell suspension is assessed for cell viability. In some embodiments, the cells are then seeded into suspension bioreactors for further expansion. In some embodiments, the cells are further cultured in suspension as aggregates for about 28 to about 68 days with daily medium exchange. In some embodiments, the cells are cultured in suspension as aggregates for about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, or about 68 days with daily medium exchange. In some embodiments, the cells are further cultured in suspension as aggregates for about 48 days with daily medium exchange. In some embodiments, DAPT is added. In some embodiments, lactate concentration, pH, dissolved oxygen, cell patterning, and/or karyotypes are monitored.

In some embodiments, cells are then harvested and triturated into a single cell suspection and strained. In some embodiments, the cells are assessed for viable cell concentration. In some embodiments, the cells are seeded onto laminin 521-coated vessels. In some embodiments, isethionate is added.

In some embodiments, the cells are then harvested, centrifuged, resuspended, pooled, and strained. In some embodiments, the cells are enriched for CD133+ cells, pooled, and assessed for viable cell concentration. In some embodiments, the culture is further maintained for about 1, about 2, about 3, or about 4 days to generate cell aggregates. In some embodiments, the cells are then pooled and strained, and a drug substance is obtained.

In some embodiments, the drug substance is formulated into a drug product by resuspension in a DMSO-containing cryopreservation medium and aseptic transfer into a vial. In some embodiments, the drug product is then frozen using liquid nitrogen.

In some embodiments, the drug product is formulated into a dose for administration by thawing, washing, and diluting with a vehicle solution. In some embodiments, the vehicle solution is Balanced Salt Solution (BSS®) Sterile Irrigating Solution supplemented with 0.2% Human Serum Albumin (HSA). In some embodiments, aggregates are enriched with slow-rate centrifugation. In some embodiments, a live cell concentration and/or percentage viability is assessed for the dose for administration. In some embodiments, the concentration of cells may be adjusted by dilution or centrifugation with resuspension. In some embodiments, the dose for administration is further assessed for sterility, endotoxin levels, and/or visual appearance.

In some embodiments, to prepare a dose for administration (DfA), the drug product (DP) may be formulated by thawing, washing, and diluting with a vehicle solution. The vehicle solution may comprise Balanced Salt Solution (BSS®) Sterile Irrigating Solution supplemented with 0.2% Human Serum Albumin (HSA). In some embodiments, the number of DP vials to thaw may be calculated based on the target dose concentration and live cell concentration of the DP. In some embodiments, after the cells are pooled and the cryopreservation buffer is diluted, the aggregates may be enriched via slow-rate centrifugation, and the supernatant may be removed. In some embodiments, a fixed volume vehicle solution may be added to the tube to resuspend the cells. In some embodiments, the enriched aggregate suspension may be centrifuged again, and the supernatant may be removed. In some embodiments, the cell pellet may be resuspended again in a fixed volume of vehicle solution, the tube may be centrifuged again, and the supernatant may be removed to a targeted volume. In some embodiments, the pelleted cells may be resuspended within the targeted volume to create the bulk dose. In some embodiments, the master dose may be prepared at the target live cell concentration for further DfA container closure fill. In some embodiments, about one adjustment of the live cell concentration to within the allowable range may be performed. In some embodiments, a calculated volume of the bulk dose may be transferred to a new tube and re-suspended in a designated volume of vehicle solution based on the bulk dose live cell concentration result. In some embodiments, this step may formulate the target master dose based on the intended clinical dose level. In some embodiments, the master dose may be then sampled for total and live cell concentration to enable calculation of percent viability. In some embodiments, if the enumeration does not meet the assay validity criteria, about one re-sampling may be performed. In some embodiments, if the repeated master dose enumeration fails, the run may be terminated. In some embodiments, when a valid result is obtained, if the live cell concentration is not within the allowable range for the intended clinical dose, the master dose concentration may be adjusted. In some embodiments, if the viability does not meet the specification, the run may be terminated. In some embodiments, if both criteria are met, the adjusted master dose may be filled into the DfA container closure.

In some embodiments, the master dose may be adjusted to within the allowable live cell concentration range. In some embodiments, if the master dose needs to be diluted to a lower live cell concentration, vehicle solution may be added to the master dose and mixed. If the master dose needs to be concentrated, the cells are pelleted via centrifugation and a calculated volume of supernatant is removed from the dose. In some embodiments, the adjusted master dose may be then mixed to resuspend the cells and sampled for total and live cell concentration to enable calculation of percent viability. In some embodiments, if the enumeration does not meet the assay validity criteria, about one re-sampling may be performed. In some embodiments, if the repeated adjusted master dose enumeration fails, the run may be terminated. In some embodiments, when a valid result is obtained, if the live cell concentration is not within the allowable range for the intended clinical dose or if the viability does not meet the specification, the run may be terminated. In some embodiments, if both criteria are met, the adjusted master dose may be filled into the DfA container closure.

In some embodiments, the time at which the final master dose was prepared may be recorded, and the expiry time and date may be calculated from this value and recorded. In some embodiments, the final master dose may be used to fill two doses of 50 μL each (1 clinical dose and 1 back-up dose) into pre-inspected tubes after uniformly resuspending the master dose by gentle pipetting. In some embodiments, six 1.5 mL aliquots of vehicle solution may be filled into pre-inspected tubes. In some embodiments, the final live cell concentration and percent viability determined from enumeration of the master dose may be recorded for release of the DfA. In some embodiments, additional samples of the master dose and the vehicle solution may be taken for sterility and endotoxin testing. In some embodiments, appearance may be evaluated by visual inspection of the final DfA and vehicle solution in their respective container closure systems. In some embodiments, interim release of the DfA for patient administration is based upon appearance, viability, live cell concentration, and endotoxin testing whereases interim release of vehicle solution for patient administration is based upon appearance and endotoxin testing. In some embodiments, primary container labels may be applied across the top of each respective DfA and vehicle solution tube such that the ends of the labels adhere to the rack the tube is contained in, and each rack containing tubes is packaged in a sterile sampling bag and maintained at about 1-8° C. In some embodiments, the packaged racks, DfA and vehicle solution tubes are transferred to overnight storage until interim QC testing and interim release of the DfA and vehicle solution have been completed. In some embodiments, after overnight storage, DfA and vehicle solution packages may be transferred to a temperature-controlled package which may be monitored during transportation to the surgical administration site.

In some embodiments, a percentage viability is assessed for a dose for administration to quantify the percentage of live cells to support product quality. In some embodiments, assessments of percentage viability involve two independent cell counting assays: one for determining the live cell concentration and another for the total cell concentration. From these measurements, the percentage of viable cells (e.g., percentage cell viability) may be calculated as the live cell concentration divided by the total cell concentration, multiplied by 100.

In some embodiments, to determine live cell concentration, cell aggregates are dissociated both enzymatically and manually. In some embodiments, to determine live cell concentration, cell aggregates are dissociated enzymatically. For example, enzymatic dissociation may be achieved using the reagent 10× TrypLE™ Select. To determine live cell concentration, cell aggregates may be dissociated manually including, for example, using trituration. The enzymatic reaction may then be quenched, and live cells quantified using a fluorescence dye such as acridine orange/propidium iodide (AO/PI). To measure total cell concentration, cells may be lysed with Solution 10™ lysis buffer. Total nuclei may then be quantified using a fluorescence dye such as acridine orange/propidium iodide (AO/PI).

In some embodiments, a percentage cell viability of a dose for administration comprising PRP cells in a suspended form may be calculated using automated cell counting including, for example, with acridine orange and/or propidium iodide using, for example, a Cellaca™ MX (Revvity, Waltham, MA, Part No.: MX-AOPI) according to the manufacturer's instructions. In some embodiments, a percentage cell viability of a dose for administration comprising PRP cells in a single cell form or an aggregate form may be calculated using automated cell counting including, for example, with an IncuCyte® Live-Cell Analysis System (Sartorius; Gottingen, Germany; Cat. No. SX5) according to the manufacturer's instructions. In some embodiments, a percentage cell viability of a dose for administration comprising PRP cells in a single cell form or an aggregate form may be calculated using automated cell counting including, for example, with an imaging based technique.

In some embodiments, cells may be counted after contact with a dissociation reagent, a cell lysis reagent, a cell staining reagent, and/or a nuclei staining reagent. In some embodiments, cells are counted after contact with a cell lysis reagent and a nuclei staining reagent. In some embodiments, cells are counted after contact with a dissociation reagent and a cell staining reagent. In some embodiments, cells are counted after contact with a nuclei staining reagent. In some embodiments, cells are counted after contact with a cell staining reagent.

The percentage viability of cells within a composition (e.g., a composition comprising cell aggregates) may be calculated by determining a ratio between the number of live cells and the number of total cells within the composition including, for example an aliquot obtained from the composition. For example, the percentage viability may be calculated by dividing the number of live cells by the number of total cells. In further embodiments, the number of total cells in a composition may be calculated by dividing the number of live cells by the percentage viability.

In some embodiments, the dose for administration and/or a cell product comprising cell aggregates displays a percentage viability of at least about 40%. In some embodiments, the dose for administration displays a percentage viability of at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%). In some embodiments, the dose for administration displays a percentage viability of at least about 40% and about 100% (e.g., about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%).

In some embodiments, an aggregate analysis test may be used to confirm the presence and quantity of cell aggregates. The aggregate analysis assay may characterize the size of aggregates in a cell product comprising cell aggregates, or to characterize the fraction of cells present as aggregates. For example, a vial of cryopreserved cell product comprising cell aggregates in cryopreservant (e.g., CryoSTOR® CS10 solution) may be suspended in vehicle solution (e.g., BSS+HSA). The cell product comprising cell aggregates may be thawed and mixed by gently inverting. A syringe containing vehicle solution may then be attached to the vial and the vehicle solution injected into the vial. The suspension of cell product comprising cell aggregates may then be drawn into the syringe using and subsequently transferred to a conical tube. An additional volume of vehicle solution may then be added to the aggregate suspension in the conical tube. The vial may be washed with vehicle solution to ensure all cells were collected, and the wash added to the conical tube.

The aggregate suspension may then be centrifuged at room temperature and most of the supernatant removed. The aggregate suspension may be triturated and aliquoted into tubes for particle size analysis, percentage aggregate analysis, and/or imaging analysis. In some embodiments, a particle size analyzer is used to characterize the size of aggregates and/or the fraction of cells present as aggregates. In some embodiments, measurements are acquired for particle diameter size (measured in μm) and/or aggregate percentage (measured by aggregate volume divided by total cell volume, which includes single cells). In some embodiments, a DMi1 inverted microscope is used to image the aggregate suspension. In some embodiments, the aggregate suspension is mixed and subsequently imaged with 20× magnification.

In some embodiments, the ratio of aggregates to total cells is then calculated as percentage biomass. In some embodiments, the cell product comprising cell aggregates displays a percentage biomass of at least about 70%. In some embodiments, the cell product comprising cell aggregates display a percentage biomass of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%). In some embodiments, the cell product comprising cell aggregates displays a percentage biomass between about 40% and about 100% (e.g., about 70% to about 100%, about 70% to about 90%, about 70% to about 80%, about 80% to about 100%, about 80% to about 90%, or about 90% to about 100%).

In some embodiments, the stability of the DfA is assessed by one or more of: viable cell concentration, percentage viability, and/or percentage aggregate biomass. In some embodiments, the stability of the DfA is assessed by viable cell concentration. In some embodiments, the stability of the DfA is assessed by percentage viability. In some embodiments, the stability of the DfA is assessed by percentage aggregate biomass.

In some embodiments, the DfA is stable at about 1-8° C. for about 0 hours to about 50 hours. In some embodiments, the DfA is stable at about 1-8° C. for about 0 hours, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, or about 50 hours. In some embodiments, the DfA is stable after a hold at about 1-8° C. for about 0-50 hours, about 10-40 hours, about 20-30 hours, about 4-31 hours, about 4-7 hours, about 24-27 hours, or about 28-31 hours. In some embodiments, the DfA is stable at about 1-8° C. for about 4-31 hours. In some embodiments, the DfA is stable at about 1-8° C. for about 4-7 hours. In some embodiments, the DfA is stable at about 1-8° C. for about 24-27 hours. In some embodiments, the DfA is stable at about 1-8° C. for about 28-31 hours.

In some embodiments, there is no change (e.g., no substantial change) in one or more of viable cell concentration, percentage viability, and/or percentage aggregate biomass of the DfA at about 1-8° C. for about 4-31 hours. In some embodiments, there is no change (e.g., no substantial change) in the viable cell concentration of the DfA at about 1-8° C. for about 4-31 hours. In some embodiments, there is no change (e.g., no substantial change) in the percentage viability of the DfA at about 1-8° C. for about 4-31 hours. In some embodiments, there is no change (e.g., no substantial change) in the percentage aggregate biomass of the DfA at about 1-8° C. for about 4-31 hours. In some embodiments, there is no change (e.g., no substantial change) in one or more of viable cell concentration, percentage viability, and/or percentage aggregate biomass of the DfA at about 1-8° C. for about 4-7 hours. In some embodiments, there is no change (e.g., no substantial change) in the viable cell concentration of the DfA at about 1-8° C. for about 4-7 hours. In some embodiments, there is no change (e.g., no substantial change) in the percentage viability of the DfA at about 1-8° C. for about 4-7 hours. In some embodiments, there is no change (e.g., no substantial change) in the percentage aggregate biomass of the DfA at about 1-8° C. for about 4-7 hours. In some embodiments, there is no change (e.g., no substantial change) in one or more of viable cell concentration, percentage viability, and/or percentage aggregate biomass of the DfA at about 1-8° C. for about 24-27 hours. In some embodiments, there is no change (e.g., no substantial change) in the viable cell concentration of the DfA at about 1-8° C. for about 24-27 hours. In some embodiments, there is no change (e.g., no substantial change) in the percentage viability of the DfA at about 1-8° C. for about 24-27 hours. In some embodiments, there is no change (e.g., no substantial change) in the percentage aggregate biomass of the DfA at about 1-8° C. for about 24-27 hours. In some embodiments, there is no change (e.g., no substantial change) in one or more of viable cell concentration, percentage viability, and/or percentage aggregate biomass of the DfA at about 1-8° C. for about 28-31 hours. In some embodiments, there is no change (e.g., no substantial change) in the viable cell concentration of the DfA at about 1-8° C. for about 28-31 hours. In some embodiments, there is no change (e.g., no substantial change) in the percentage viability of the DfA at about 1-8° C. for about 28-31 hours. In some embodiments, there is no change (e.g., no substantial change) in the percentage aggregate biomass of the DfA at about 1-8° C. for about 28-31 hours.

In some embodiments, the cell product comprising cell aggregates (e.g., drug product, master dose, or dose for administration) may be assessed including, for example, for dose assurance. The dose assurance may be assessed under simulated use conditions. Simulated use conditions may include, but are not limited to, the impact of passage through a cannula (e.g., PolyTip Funnel Cannula 25G/33G; MedOne, #3510) and/or syringe (e.g., MicroDose Injection Kit; MedOne #3275), the impact of passage through a delivery device (e.g., Constellation Vision System and/or tubing from VFC Pak; Alcon #8065750957) and/or general loss of cell product during loading and delivery. Potential sources of cell loss may include cell adhesion or loss during pipetting of the DfA, cell adhesion and loss within the DfA tube during loading of the DfA, loss to device dead volume, or decrease in viability from injection with the delivery system.

In some embodiments, the assessment of the impact of such simulated use conditions includes measurements of one or more of viable cell concentration, total cell concentration, and/or percentage viability. In some embodiments, the assessment of the impact of such simulated use conditions includes measurements of viable cell concentration. In some embodiments, the assessment of the impact of such simulated use conditions includes measurements of total cell concentration. In some embodiments, the assessment of the impact of such simulated use conditions includes measurements of percentage viability.

In some embodiments, the impact of simulated use conditions is determined by comparing a sample of cells that has not been passed through the delivery device (e.g., cannula, syringe, system, and/or tubing) to a sample of cells that has been passaged through the delivery device. In some embodiments, under simulated use conditions as described herein, the cell product comprising aggregates does not show a substantial change as compared to cell product comprising aggregates that has not been treated under simulated use conditions in one or more of viable cell concentration, total cell concentration, and/or percentage viability. In some embodiments, if the cell product comprising cell aggregates does not show a substantial change in one or more of viable cell concentration, total cell concentration, and/or percentage viability, then the cell product comprising cell aggregates is considered suitable for dose assurance purposes.

In some embodiments, the cell product comprising cell aggregates (e.g., drug product, master dose, or dose for administration) is assessed including, for example, for dose assurance. The dose assurance may be assessed under simulated worst-case scenario conditions. Simulated worst-case scenario conditions may include, but are not limited to, extended storage at about 1-8° C. (e.g., more than 24 hours), extended transport conditions (e.g., prolonged agitation), extended in-device hold time, and/or injection with the injection pressure set to the maximum setting (e.g., 10 psi). In some embodiments, the assessment of the impact of such conditions includes measurements of one or more of percentage cell viability, viable cell count, percentage aggregate biomass, and/or extended culture viable cell counts. In some embodiments, the assessment of the impact of such conditions includes measurement of percentage cell viability. In some embodiments, the assessment of the impact of such conditions includes measurement of viable cell count. In some embodiments, the assessment of the impact of such conditions includes measurement of percentage aggregate biomass. In some embodiments, the assessment of the impact of such conditions includes measurement of extended culture viable cell counts.

In some embodiments, the impact of simulated worst-case scenario conditions is determined by comparing a sample of cells that has not been exposed to such conditions (e.g., extended storage at about 1-8° C., extended transport conditions, extended in-device hold time, or passage through the delivery device with injection at 10 psi) to a sample of cells that has been exposed to simulated worst-case scenario conditions. In some embodiments, under simulated worst-case scenario conditions as described herein, the cell product comprising aggregates does not show a substantial change as compared to cell product comprising aggregates that has not been treated under simulated worst-case scenario conditions in one or more of percentage cell viability, viable cell count, percentage aggregate biomass, and/or extended culture viable cell counts. In some embodiments, if the cell product comprising cell aggregates does not show a substantial change in one or more of percentage cell viability, viable cell count, percentage aggregate biomass, and/or extended culture viable cell counts, then the cell product comprising cell aggregates is considered suitable for worst-case scenario dose assurance purposes.

In some embodiments, the cell product comprising cell aggregates prepared as a dose for administration is assessed for dose assurance under worst-case scenario conditions. In some embodiments, the worst-case scenario conditions include exposure to about 1-8° C. for about 0 hours to about 48 hours. In some embodiments, the worst-case scenario conditions include exposure to about 1-8° C. for about 0 hours, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours. In some embodiments, the worst-case scenario conditions include exposure to about 1-8° C. for about 0-10 hours, about 5-15 hours, about 10-20 hours, about 15-25 hours, about 18-24 hours, about 20-30 hours, about 25-35 hours, about 30-40 hours, about 35-45 hours, or about 40-48 hours. In some embodiments, the worst-case scenario conditions include exposure to about 1-8° C. for about 18 to about 24 hours. In some embodiments, the worst-case scenario conditions include exposure to about 1-8° C. for about 28 hours.

In some embodiments, the worst-case scenario conditions include agitation meant to mimic transport prior to loading the cell composition comprising cell aggregates (e.g., the dose for administration) into a delivery device and/or prior to administration. In some embodiments, the worst-case scenario conditions include agitation for about 0 to about 100 minutes. In some embodiments, the worst-case scenario conditions include agitation for about 0 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes about 79 minutes, about 80 minutes, about 85 minutes, about 90 minutes, about 95 minutes, or about 100 minutes. In some embodiments, the worst-case scenario conditions include agitation for about 0-15 minutes, about 5-20 minutes, about 10-25 minutes, about 15-30 minutes, about 20-35 minutes, about 25-40 minutes, about 30-45 minutes, about 35-50 minutes, about 40-55 minutes, about 45-60 minutes, about 50-65 minutes, about 55-70 minutes, about 60-75 minutes, about 65-80 minutes, about 70-85 minutes, about 75-90 minutes, about 80-95 minutes, or about 85-100 minutes. In some embodiments, the worst-case scenario conditions include agitation for about 60 minutes to about 75 minutes.

In some embodiments, the worst-case scenario conditions include aspiration of a composition comprising cell aggregates (e.g., the dose for administration) into a syringe (MicroDose Injection Kit; MedOne #3275) and cannula (PolyTip Funnel Cannula 25G/33G; MedOne, #3510), e.g., the delivery device, prior to administration in the vertical orientation. In some embodiments, the aspiration of the composition comprising cell aggregates is aspirated into the delivery device via tubing (VFC Pak; Alcon #8065750957) with a maximum aspiration pressure setting of about 350 mmHg. In some embodiments, the worst-case scenario conditions include an in-device hold time in a vertical orientation of about 0 minutes to about 25 minutes. In some embodiments, the worst-case scenario conditions include an in-device hold time in a vertical orientation of about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, or about 25 minutes. In some embodiments, the worst-case scenario conditions include an in-device hold time in a vertical orientation of about 0-2 minutes, about 1-3 minutes, about 2-4 minutes, about 3-5 minutes, about 4-6 minutes, about 5-7 minutes, about 6-8 minutes, about 7-9 minutes, about 8-10 minutes, about 9-11 minutes, about 10-12 minutes, about 11-13 minutes, about 12-14 minutes, about 13-15 minutes, about 14-16 minutes, about 15-17 minutes, about 16-18 minutes, about 17-19 minutes, about 18-20 minutes, about 19-21 minutes, about 20-22 minutes, about 21-23 minutes, about 22-24 minutes, or about 23-25 minutes. In some embodiments, the worst-case scenario conditions include an in-device hold time in a vertical orientation of about 15-17 minutes.

In some embodiments, the worst-case scenario conditions include injection of the composition comprising cell aggregates (e.g., the dose for administration) from a syringe and cannula (e.g., the delivery device) into a tube or cell culture plate at an injection pressure of about 0.5 psi to about 12.5 psi. In some embodiments, the worst-case scenario conditions include an injection pressure of about 0.5 psi, about 1.0 psi, about 1.5 psi, about 2.0 psi, about 2.5 psi, about 3.0 psi, about 3.5 psi, about 4.0 psi, about 4.5 psi, about 5.0 psi, about 5.5 psi, about 6.0 psi, about 6.5 psi, about 7.0 psi, about 7.5 psi, about 8.0 psi, about 8.5 psi, about 9.0 psi, about 9.5 psi, about 10.0 psi, about 10.5 psi, about 11.0 psi, about 11.5 psi, about 12.0 psi, or about 12.5 psi. In some embodiments, the worst-case scenario conditions include an injection pressure of about 0.5-1.5 psi, about 1.0-2.0 psi, about 1.5-2.5 psi, about 2.0-3.0 psi, about 2.5-3.5 psi, about 3.0-4.0 psi, about 3.5-4.5 psi, about 4.0-5.0 psi, about 4.5-5.5 psi, about 5.0-6.0 psi, about 5.5-6.5 psi, about 6.0-7.0 psi, about 6.5-7.5 psi, about 7.0-8.0 psi, about 7.5-8.5 psi, about 8.0-9.0 psi, about 8.5-9.5 psi, about 9.0-10.0 psi, about 9.5-10.5 psi, about 10.0-11.0 psi, about 10.5-11.5 psi, about 11.0-12.0 psi, or about 11.5-12.5 psi. In some embodiments, the worst-case scenario conditions include an injection pressure of about 10.0 psi.

In some embodiments, the composition comprising cell aggregates (e.g., the dose for administration) from a syringe and cannula (e.g., the delivery device) is injected at a pressure of 10 psi into a tube or cell culture plate which contains about 5 uL to about 30 uL of vehicle solution to mimic injection into the fluid environment of the eye. In some embodiments, the vehicle solution comprises BSS supplemented with HSA as described herein. In some embodiments, the tube or culture plate contains about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, or about 30 μL of vehicle solution. In some embodiments, the tube or culture plate contains about 5-10 μL, about 10-15 μL, about 15-20 μL, about 20-25 μL, or about 35-30 μL of vehicle solution. In some embodiments, the tube or cell culture plate contains about 10 μL of vehicle solution.

In an aspect, provided herein is a method for assessing whether a dose for administration comprising PRPs is compatible with a system for administration, the method comprising: a) exposing the PRPs to about 1-8° C.; b) agitating the PRPs; c) aspirating the PRPs into a cannula and syringe; d) holding the PRPs in the syringe in a vertical orientation; e) injecting the PRPs from the cannula and syringe into a tube and/or cell culture plate; and f) measuring percentage aggregate biomass, percentage cell viability, and/or viable cell concentration of the PRPs in the tube and/or cell culture plate, wherein the dose for administration comprising PRPs is compatible with a system for administration if there is no change (e.g., no substantial change) in percentage aggregate biomass, percentage cell viability, and/or viable cell concentration of the dose for administration comprising PRPs compared to a composition comprising control PRPs. In some embodiments, the composition comprising control PRPs refers to a population of PRPs that has not been subjected to steps a) to e).

In some embodiments, the composition comprising control PRPs refers to a population of PRPs that has not been exposed to about 1-8° C., has not been agitated, has not been aspirated into a cannula and syringe, has not been held in the syringe in a vertical orientation, and/or has not been injected from the cannula and syringe into a tube and/or culture plate. In some embodiments, the composition comprising control PRPs refers to a population of PRPs that has not been exposed to about 1-8° C. In some embodiments, the composition comprising control PRPs refers to a population of PRPs that has not been agitated. In some embodiments, the composition comprising control PRPs refers to a population of PRPs that has not been aspirated into a cannula and syringe. In some embodiments, the composition comprising control PRPs refers to a population of PRPs that has not been held in the syringe in a vertical orientation. In some embodiments, the composition comprising control PRPs refers to a population of PRPs that has not been injected from the cannula and syringe into a tube and/or culture plate.

In some embodiments, the amount of the PRPs in the composition is verified (e.g., within acceptable limits) if the percentage aggregate biomass is at least about 70%. In some embodiments, dose assurance is confirmed if the percentage cell viability is at least about 40%. In some embodiments, dose assurance is confirmed if the viable cell concentration does not change (e.g., does not substantially change) from its initial clinical target dose. In some embodiments, dose assurance is confirmed wherein one or more of: the percentage aggregate biomass is at least about 70%, the percentage cell viability is at least about 40%, and the viable cell concentration does not change (e.g., does not substantially change) from its initial clinical target dose.

In some embodiments, a dose for administration comprising PRPs is compatible with a system for administration if the change in percentage aggregate biomass, percentage cell viability, and/or viable cell concentration of the PRPs in the tube and/or cell culture plate is less than about 50% compared to a composition comprising control PRPs. In some embodiments, a dose for administration comprising PRPs is compatible with a system for administration if the change in percentage aggregate biomass, percentage cell viability, and/or viable cell concentration of the PRPs in the tube and/or cell culture plate is less than about 50%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% compared to a composition comprising control PRPs.

In some embodiments, a dose for administration comprising PRPs is compatible with a system for administration if the change in percentage aggregate biomass, percentage cell viability, and/or viable cell concentration of the PRPs in the tube and/or cell culture plate is less than about 40% compared to a composition comprising control PRPs. In some embodiments, a dose for administration comprising PRPs is compatible with a system for administration if the change in percentage aggregate biomass, percentage cell viability, and/or viable cell concentration of the PRPs in the tube and/or cell culture plate is less than about 30% compared to a composition comprising control PRPs. In some embodiments, a dose for administration comprising PRPs is compatible with a system for administration if the change in percentage aggregate biomass, percentage cell viability, and/or viable cell concentration of the PRPs in the tube and/or cell culture plate is less than about 20% compared to a composition comprising control PRPs. In some embodiments, a dose for administration comprising PRPs is compatible with a system for administration if the change in percentage aggregate biomass, percentage cell viability, and/or viable cell concentration of the PRPs in the tube and/or cell culture plate is less than about 10% compared to a composition comprising control PRPs.

In some embodiments, the resulting cell population comprises AIPL1+ and RCVRN+ iPRP cells subdivided by NR2E3+ rod-committed PRP cells and PPP4R4+ cone-committed iPRP cells with minimally detected CHX10+, PAX6+, TYRP1+, and Ki67+ off-target cells, minimally detected VIM gene expression, and no detectable LNCPRESS2 and AC009446.1 gene expression.

Cells, such as PRP cells or PSCs, can be cultured with the nutrients necessary to support the growth of each specific population of cells. Generally, the cells are cultured in growth media including a carbon source, a nitrogen source, and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium Additionally, the minimal essential media may be supplemented with additives such as horse, calf, or fetal bovine serum Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully defined and feeder-free media.

Accordingly, the single cell PSCs are generally cultured in a fully defined culture medium after plating. In certain aspects, about 18 to 24 hours after seeding, the medium is aspirated and fresh medium, such as E8™ medium, is added to the culture. In certain aspects, the single cell PSCs are cultured in the fully defined culture medium for about 1, about 2 or about 3 days after plating. Preferably, the single cells PSCs are cultured in the fully defined culture medium for about 2 days before proceeding with the differentiation process.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically defined Lipid concentrated (Gibco), and GLUTAMAX™ (Gibco).

Other culturing conditions can be appropriately defined. For example, the culturing temperature can be about 30 to about 40° C., for example, at least about 31, at least about 32, at least about 33, at least about 34, at least about 35, at least about 36, at least about 37, at least about 38, or at least about 39° C. but particularly not limited to them. In one embodiment, the cells are cultured at 37° C. The CO₂ concentration can be about 1 to about 10%, for example, about 2 to 5%, or any range derivable therein. The oxygen tension can be at least, up to, or at least about 1, at least about 2, at least about 3, at least about 4, at least about 5, at least about 6, at least about 7, at least about 8, at least about 9, at least about 10, at least about 20%, or any range derivable therein.

In some embodiments, the photoreceptor precursor cells produced by the methods disclosed herein can be cryopreserved, see for example, PCT Publication No. WO 2012/149484 A2, which is incorporated by reference herein. The cells can be cryopreserved with or without a substrate. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored about 48 hours to about one week. In yet other embodiments, the cells are stored for about 1, about 2, about 3, about 4, about 5, about 6, about 7, or about 8 weeks. In further embodiments, the cells are stored for about 1, about 2, about 3, about 4, about 5, about 6, about 7, about 8, about 9, about 10, about 11, or about 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80 or serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8%- to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C./minute during cryopreservation. In some embodiments, the cryopreservation temperature is about −80° C. to about −180° C., or about −125° C. to about −140° C. In some embodiments, the cells are cooled to about 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

Alternatively, the cells may be cryopreserved as aggregates without dissociation into a single cell suspension. For example, single cells may be allowed to re-aggregate in tissue culture flasks for two days in minimal medium. Aggregates may be pooled and a sample aliquot obtained for cell counts. Following a series of washes, aggregates can be resuspended in CryoSTOR® CS10 Freeze Medium and the aggregate suspensions may be transferred to liquid nitrogen storage vials, such as at 25×10⁶ aggregated cell products/vial.

In some embodiments, inhibitors or other additives may be added to culture media, including WNT pathway inhibitors, BMP pathway inhibitors, TGFβ pathway inhibitors, MEK inhibitors, gamma secretase inhibitors, and/or cyclin dependent kinase inhibitors.

### Kits

In some embodiments, a kit that can include, for example, one or more media and components for the production of the composition comprising the PRP cells is provided. Such formulations may comprise a variety of retinal differentiation and/or trophic factors, in a form suitable for combining with photoreceptor precursor or photoreceptor cells. The reagent system may be packaged either in aqueous media or in lyophilized form, where appropriate. The container means of the kits will generally include at least one vial, test tube, flask, bottle, syringe, or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### Illustration of Subject Technology as Clauses

Various examples of aspects are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: A composition of cells comprising photoreceptor precursor cells (PRPs), wherein the composition comprises: NR2E3+ cells and between at least about 20% to about 60% PPP4R4+ cells; wherein the PRPs optionally comprise less than or equal to about 0.5 copies of VIM per copy of SDHA.

Clause 2: The composition according to clause 1, wherein at least about 20% to about 60% of the cells are PPP4R4+, wherein the PRPs are substantially NR2E3+, and wherein the cells comprise less than or equal to about 0.5 copies of VIM per copy of SDHA.

Clause 3: The composition according to clause 1 or 2, wherein the cells comprise: at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

Clause 4: The composition according to clause 3, wherein percentage NR2E3+, PPP4R4+, AIPL1+, RCVRN+, PAX6+, CHX10+, TYRP1+, and/or Ki67+ cells is determined by flow cytometry.

Clause 5: The composition according to any one of clauses 1-4, wherein copies of VIM per copy of SDHA is determined by droplet digital polymerase chain reaction (ddPCR).

Clause 6: The composition according to any one of clauses 1-5, wherein the cells have a percentage viability of at least about 50%.

Clause 7: The composition according to clause 6, wherein the percentage viability is determined by automated cell counting using acridine orange and propidium iodide.

Clause 8: The composition according to any one of clauses 1-7, wherein the cells are of human origin.

Clause 9: The composition according to any one of clauses 1-8, wherein the cells are derived from pluripotent stem cells (PSCs) or PSC-derived cells, optionally induced PSCs (iPSCs).

Clause 10: A composition of cells consisting of photoreceptor precursor cells (PRPs) and, optionally, a pharmaceutically acceptable excipient thereof, wherein the cells consist of: NR2E3+ cells and between at least about 20% to about 60% PPP4R4+ cells; and wherein the PRPs comprise less than or equal to about 0.5 copies of VIM per copy of SDHA.

Clause 11: The composition according to clause 10, wherein the cells consist of: at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and less than or equal to about 0.5% Ki67+ cells.

Clause 12: The composition according to clause 11, wherein percentage NR2E3+, PPP4R4+, AIPL1+, RCVRN+, PAX6+, CHX10+, TYRP1+, and/or Ki67+ cells is determined by flow cytometry.

Clause 13: The composition according to any one of clauses 10-12, wherein copies of VIM per copy of SDHA is determined by droplet digital polymerase chain reaction (ddPCR).

Clause 14: The composition according to any one of clauses 10-13, wherein the cells have a percentage viability of at least about 40%.

Clause 15: The composition according to clause 14, wherein the percentage viability is determined by automated cell counting using acridine orange and propidium iodide.

Clause 16: The composition according to any one of clauses 10-16, wherein the cells are of human origin.

Clause 17: The composition according to any one of clauses 10-16, wherein the cells are derived from pluripotent stem cells (PSCs) or PSC-derived cells, optionally induced PSCs (iPSCs).

Clause 18: A composition of cells comprising PRPs, wherein the composition comprises cell aggregates, wherein the cell aggregates have a median diameter of about 30 μm to about 60 μm.

Clause 19: The composition of cells comprising PRPs according to clause 18, wherein the median diameter of the cell aggregates is determined by a particle size analyzer.

Clause 20: The composition of cells comprising PRPs according to clause 18 or 19, wherein each cell is NR2E3+ or PPP4R4+.

Clause 21: The composition of cells comprising PRPs according to clause 18, wherein the cells comprise between at least about 20% to about 60% PPP4R4+ cells and wherein the cells comprise less than or equal to about 0.5 copies of VIM per copy of SDHA.

Clause 22: The composition of cells comprising PRPs according to clause 20 or 21, wherein a percentage of NR2E3+ and/or PPP4R4+ cells is determined by flow cytometry.

Clause 23: The composition of cells comprising PRPs according to clause 21, wherein copies of VIM per copy of SDHA is determined by ddPCR.

Clause 24: The composition of cells comprising PRPs according to any one of clauses 1-23, wherein the cells are in a single cell form, an aggregate form, or a suspended form.

Clause 25: The composition of cells comprising PRPs according to any one of clauses 1-23, wherein the cells are in a single cell form or an aggregate form.

Clause 26: The composition of cells comprising PRPs according to any one of clauses 1-23, wherein the cells are in a suspended form.

Clause 27: A composition of cells comprising PRPs according to any one of clauses 1-26 and a pharmaceutically acceptable carrier.

Clause 28: The composition according to any one of clauses 1-27, wherein the cells are suitable for grafting to a subretinal space of a subject's eye.

Clause 29: A method of identifying a cell population for treatment of an eye disease or disorder, the method comprising: assaying for PPP4R4+ cells in a cell population, wherein the cell population comprises between at least about 20% to about 60% PPP4R4+ cells, and wherein and the cells comprise less than or equal to 0.5 copies of VIM per copy of SDHA.

Clause 30: The method according to clause 29, wherein the method further comprises: assaying for AIPL1+ and/or RCVRN+ cells in the cell population, wherein the cells are substantially NR2E3+, at least about 90% of the cells in the cell population are AIPL1+; at least about 90% of the cells in the cell population are RCVRN+; no more than about 7% of the cells are PAX6+; no more than about 7% of the cells are CHX10+; no more than about 0.5% of the cells are TYRP1+; or no more than about 0.5% of the cells are Ki67+.

Clause 31: The method according to clause 30, wherein a percentage of NR2E3+, PPP4R4+, AIPL1+, RCVRN+, PAX6+, CHX10+, TYRP1+, and/or Ki67+ cells is determined by flow cytometry.

Clause 32: The method according to any one of clauses 29-31, wherein copies of VIM per copy of SDHA is determined by ddPCR.

Clause 33: The method according to of any one of clauses 29-32, wherein the cell population has a percentage viability of at least about 40%.

Clause 34: The method according to clause 33, wherein the percentage viability is determined by an automated cell counting using acridine orange and propidium iodide.

Clause 35: The method according to any one of clauses 29-34, wherein the cell population is of human origin.

Clause 36: The method according to any one of clauses 29-35, wherein the cell population is derived from PSCs or PSC-derived cells, optionally iPSCs.

Clause 37: The method according to any one of clauses 29-36, wherein the cell population is in a single cell form, an aggregate form, or a suspended form.

Clause 38: The method according to any one of clauses 29-36, wherein the cell population is in a single cell form or an aggregate form.

Clause 39: The method according to any one of clauses 29-36, wherein the cell population is in a suspended form.

Clause 40: The method according to any one of clauses 29-39, wherein the cell population is suitable for grafting to a subretinal space of a subject's eye.

Clause 41: The method of clause 29-40, wherein the eye disease or disorder is a retinal disorder.

Clause 42: The method of clause 41, wherein the retinal disorder is characterized by a loss of photoreceptors and/or a loss of photoreceptor cell function.

Clause 43: The method of clause 41 or 42, wherein the eye disease is a primary photoreceptor disease, including retinitis pigmentosa, cone-rod disease, rod-cone disease, cone dystrophy, and/or cone-rod dystrophy.

Clause 44: The method of any of clauses 41-43, wherein the eye disease of disorder is an inherited retinal disease.

Clause 45: The method of any of clauses 41-44, wherein the eye disease or disorder is Usher Syndrome.

Clause 46: A method of identifying a suitable cell population for treatment of an eye disease, the method comprising: a) obtaining a population of cells comprising PRPs; b) measuring a percentage of PPP4R4+ cells in a cell population; and c) measuring a copy number of VIM per copy SDHA; wherein the cell population is identified as a suitable cell population for treatment of an eye disease when the percentage of PPP4R4+ cells in the cell population is between about 20% and about 60%, and the copy number of VIM per copy of SDHA is less than or equal to about 0.5 copies.

Clause 47: The method according to clause 46, wherein the method further comprises: d) measuring a percentage of AIPL1+ RCVRN+, PAX6+, CHX10+, TYRP1+; and/or Ki67+ cells in the cell population, wherein the cell population is identified as a suitable cell population for treatment of an eye disease when the percentage of AIPL1+ cells is least about 90%, the percentage of RCVRN+ cells is least about 90%, the percentage of PAX6+ cells is no more than about 7%, the percentage of CHX10+ cells is no more than about 7%, the percentage of TYRP1+ cells is no more than about 0.5%, and the percentage of Ki67+ cells is no more than about 0.5%.

Clause 48: The method according to clause 47, wherein a percentage of PPP4R4+, AIPL1+, RCVRN+, PAX6+, CHX10+, TYRP1+, or Ki67+ cells is determined by flow cytometry.

Clause 49: The method according to any one of clauses 46-48, wherein copies of VIM per copy of SDHA is determined by ddPCR.

Clause 50: The method according to any one of clauses 46-49, wherein the cell population is of human origin.

Clause 51: The method according to any one of clauses 46-50, wherein the cell population is derived from PSCs or PSC-derived cells, optionally iPSCs.

Clause 52: The method according to any one of clauses 46-51, wherein the cell population is in a single cell form, an aggregate form, or a suspended form.

Clause 53: The method according to any one of clauses 46-51, wherein the cell population is in a suspended form.

Clause 54: The method according to any one of clauses 46-53, wherein the cell population is suitable for grafting to a subretinal space of a subject's eye.

Clause 55: A composition comprising human PRPs for treatment of an eye disease or disorder, wherein the composition is in suspension form, and at least a portion of the composition is aggregated.

Clause 56: The composition comprising human PRPs of clause 55, wherein the composition comprises an administered dose of about 1×10⁶, about 2×10⁶, about 3×10⁶, or about 4×10⁶ human PRPs.

Clause 57: The composition comprising human PRPs according to clause 56, wherein a dose for administration comprises: a) about 2.72×10⁷ total cells/mL to about 4.08×10⁷ total cells/mL are present in the composition; b) about 5.04×10⁷ total cells/mL to about 7.56×10⁷ total cells/mL are present in the composition; c) about 7.12×10⁷ total cells/mL to about 1.07×10⁸ total cells/mL are present in the composition; or d) about 9.44×10⁷ total cells/mL to about 1.42×10⁸ total cells/mL are present in the composition.

Clause 58: The composition according to clause 57, wherein the dose for administration further comprises balanced salt solution buffer supplemented with 0.2% human serum albumin (HSA).

Clause 59: The composition comprising human PRPs according to clause 58, wherein cells/mL is determined by automated cell counting using acridine orange and/or propidium iodide.

Clause 60: A drug product comprising human PRPs, wherein the drug product comprising the human PRPs comprises between at least about 20% to about 60% PPP4R4+ cells, wherein the drug product comprising the human PRPs are NR2E3+; and wherein the drug product comprising the human PRPs comprise less than or equal to about 0.5 copies of VIM per copy of SDHA.

Clause 61: The drug product comprising human PRPs according to clause 60, wherein the drug product further comprises human PRPs comprising: at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and/or less than or equal to about 0.5% Ki67+ cells.

Clause 62: The drug product comprising the human PRPs according to clause 61, wherein a percentage of NR2E3+, PPP4R4+, AIPL1+, RCVRN+, PAX6+, CHX10+, TYRP1+, and/or Ki67+ cells is determined by flow cytometry.

Clause 63: The drug product comprising the human PRPs according to clause 61, wherein copies of VIM per copy of SDHA is determined by ddPCR.

Clause 64: The drug product comprising the human PRPs according to any one of clauses 59-63, wherein the drug product comprising the human PRPs have a percentage viability of at least about 50%.

Clause 65: The drug product comprising the human PRPs according to clause 64, wherein the percentage viability is determined by automated cell counting using acridine orange and propidium iodide.

Clause 66: The drug product comprising the human PRPs according to any one of clauses 59-65, wherein the drug product comprising the human PRPs comprise cell aggregates wherein a median diameter of a cell aggregate is about 30 μm to about 60 μm.

Clause 67: The drug product comprising the human PRPs according to clause 66, wherein the median diameter of a cell aggregate is determined by a particle size analyzer.

Clause 68: The drug product comprising the human PRPs according to any of clauses 60-67, wherein the drug product is resuspended in media.

Clause 69: The drug product comprising the human PRPs according to any of clauses 60-67, wherein the drug product is resuspended in CryoSTOR® CS10 solution.

Clause 70: The drug product comprising the human PRPs according to any of claims 60-67, wherein the drug product is reformulated after thaw.

Clause 71: The drug product comprising the human PRPs according to any of clauses 60-70, wherein the drug product comprises about 25×10⁶ cells.

Clause 72: A composition comprising human PRPs for treatment of an eye disease or disorder, comprising a therapeutically effective amount of the composition, wherein the composition is in an aggregated form.

Clause 73: The composition comprising human PRPs according to clause 72, wherein the composition comprises an administered dose for administration of about 1×10⁶, about 2×10⁶, about 3×10⁶, or about 4×10⁶ human PRPs.

Clause 74: The composition comprising human PRPs according to clause 73, wherein a dose for administration comprises: a) about 2.72×10⁷ total cells/mL to about 4.08×10⁷ total cells/mL are present in the composition; b) about 5.04×10⁷ total cells/mL to about 7.56×10⁷ total cells/mL are present in the composition; c) about 7.12×10⁷ total cells/mL to about 1.07×10⁸ total cells/mL are present in the composition; or d) about 9.44×10⁷ total cells/mL to about 1.42×10⁸ total cells/mL are present in the composition.

Clause 75: The composition according to clause 74, wherein the dose for administration is administered within 28 hours after reformulation from a drug product.

Clause 76: The composition comprising human PRPs according to clause 75, wherein cells/mL is determined by automated cell counting using acridine orange and/or propidium iodide.

Clause 77: The composition comprising human PRPs according to any one of clauses 72-76, wherein a drug product comprising the human PRPs comprises between at least about 20% to about 60% PPP4R4+ cells, wherein the PRPs are NR2E3+; and wherein the PRPs comprise less than or equal to about 0.5 copies of VIM per copy of SDHA.

Clause 78: The composition comprising the drug product comprising the human PRPs according to clause 77, wherein the composition comprises: at least about 90% AIPL1+ cells; at least about 90% RCVRN+ cells; less than or equal to about 7% PAX6+ cells; less than or equal to about 7% CHX10+ cells; less than or equal to about 0.5% TYRP1+ cells; and/or less than or equal to about 0.5% Ki67+ cells.

Clause 79: The composition comprising the drug product comprising the human PRPs according to clause 77 or 78, wherein a percentage of NR2E3+, PPP4R4+, AIPL1+, RCVRN+, PAX6+, CHX10+, TYRP1+, and/or Ki67+ cells is determined by flow cytometry.

Clause 80: The composition comprising the drug product comprising the human PRPs according to clause 77, wherein copies of VIM per copy of SDHA is determined by ddPCR.

Clause 81: The composition comprising the drug product comprising the human PRPs of any one of clauses 72-80, wherein the composition has a percentage viability of at least about 50%.

Clause 82: The composition comprising the drug product comprising the human PRPs according to clause 81, wherein the percentage viability is determined by automated cell counting using acridine orange and propidium iodide.

Clause 83: The composition comprising the drug product comprising the human PRPs according to any one of clauses 72-82, wherein the composition further comprises cell aggregates wherein a median diameter of a cell aggregate is about 30 μm to about 60 μm.

Clause 84: The composition comprising the drug product comprising the human PRPs according to clause 83, wherein the median diameter of a cell aggregate is determined by a particle size analyzer.

Clause 85: A composition comprising human PRPs and balanced salt solution buffer supplemented with 0.2% human serum albumin (HSA).

Clause 86: The composition of clause 85, wherein the human PRPs are stable at 1-8° C. for 4-31 hours.

Clause 87: The composition of clause 85, wherein the human PRPs are stable at 1-8° C. for 4-7 hours.

Clause 88: The composition of clause 85, wherein the human PRPs are stable at 1-8° C. for 24-27 hours.

Clause 89: The composition of clause 85, wherein the human PRPs are stable at 1-8° C. for 28-31 hours.

Clause 90: The composition of any one of clauses 85 to 88, wherein stability is assessed by one or more of percentage viability, viable cell concentration, and/or percentage aggregate biomass.

Clause 91: The composition of any one of clauses 85 to 90, wherein the balanced salt solution buffer comprises 0.3 mg/mL of magnesium chloride, 0.48 mg/mL of calcium chloride, 0.75 mg/mL of potassium chloride, 1.7 mg/mL of sodium citrate, 3.9 mg/mL of sodium acetate, and/or 6.4 mg/mL of sodium chloride.

Clause 92: A method for assessing whether a dose for administration comprising PRPs is compatible with a system for administration, the method comprising: a) exposing the PRPs to 1-8° C.; b) agitating the PRPs; c) aspirating the PRPs into a cannula and syringe; d) holding the PRPs in the syringe in a vertical orientation; e) injecting the PRPs from the cannula and syringe into a tube and/or cell culture plate; and f) measuring percentage aggregate biomass, percentage cell viability, and/or viable cell concentration of the PRPs in the tube and/or cell culture plate, wherein the dose for administration comprising PRPs is compatible with a system for administration if there is no substantial change in percentage aggregate biomass, percentage cell viability, and/or viable cell concentration of the dose for administration comprising PRPs compared to a composition comprising control PRPs.

The present disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples, therefore, specifically point out the preferred embodiments of the present invention and are not to be construed as limiting in any way the remainder of the disclosure.

### EXAMPLES

### Example 1: Preparation of Cell Product

A method was developed for the differentiation of iPSCs into different stages of photoreceptor precursors (PRPs). Briefly, a population of retinal progenitor cells (RPCs) is derived from iPSCs which are then further differentiated to neural retinal progenitors (NRPs) and then PRP cells.

Briefly, iPSCs were obtained from a working cell bank (WCB). The cryopreserved WCB iPSCs were thawed and seeded onto vitronectin-coated plates and cultured overnight. Then, the iPSCs were cultured for about three to about four days with daily medium exchange. The lactate concentration was assessed to monitor cell growth.

The cells were then passaged with ethylenediaminetetraacetic acid (EDTA), harvested, and seeded into vitronectin-coated flasks. The iPSCs were cultured for an additional three days with daily medium exchange. Then, the cells were passaged using TrypLE™ for aggregate dissociation. The cells were then pooled, and the single cell suspension was analyzed for viable cell concentration. The cells were then pooled and seeded into vitronectin-coated vessels and cultured for about two days with daily medium exchange.

The cells were then cultured in vitronectin-coated vessels for about 11 to 15 days with daily medium exchange. The cells were then harvested with TrypLE™, centrifuged, resuspended, pooled, and strained to remove large clusters and isolate single cells. The resulting single cell suspension was assessed for cell viability. The cells were then seeded into suspension bioreactors for further expansion. The cells were further cultured in suspension as aggregates for about 48 days with daily medium exchange in the presence of DAPT. Throughout this process, lactate concentration, pH, dissolved oxygen, cell patterning, and/or karyotypes were monitored.

The cells were then harvested and triturated into a single cell suspection and strained. The cells were further assessed for viable cell concentration. The cells were then seeded onto laminin 521-coated vessels in the presence of isethionate. The cells were then harvested, centrifuged, resuspended, pooled, and strained. The cells were enriched for CD133+ cells, pooled, and assessed for viable cell concentration. The culture was further maintained for about two days to generate cell aggregates. The cells were then pooled and strained, producing a drug substance of cells.

The drug substance was formulated into a drug product by resuspension in a DMSO-containing cryopreservation medium and aseptic transfer into a vial. The drug product was then frozen using liquid nitrogen. The drug product comprises a cell product comprising cell aggregates.

The resulting cell population comprised AIPL1+ and RCVRN+ iPRP cells subdivided by NR2E3+ rod-committed PRP cells and PPP4R4+ cone-committed iPRP cells with minimally detected CHX10+, PAX6+, TYRP1+, and Ki67+ off-target cells, minimally detected VIM gene expression, and no detectable LNCPRESS2 and AC009446.1 gene expression. To prepare a dose for administration (DfA), the drug product (DP) was formulated by thawing, washing, and diluting with a vehicle solution. The vehicle solution comprised Balanced Salt Solution (BSS®) Sterile Irrigating Solution supplemented with 0.2% Human Serum Albumin (HSA). The number of DP vials to thaw was calculated based on the target dose concentration and live cell concentration of the DP. After the cells were pooled and the cryopreservation buffer was diluted, the aggregates were enriched via slow-rate centrifugation, and the supernatant was removed. A fixed volume vehicle solution was added to the tube to resuspend the cells. The enriched aggregate suspension was centrifuged again, and the supernatant was removed. The cell pellet was resuspended again in a fixed volume of vehicle solution, the tube was centrifuged again, and the supernatant was removed to a targeted volume. The pelleted cells were resuspended within the targeted volume to create the bulk dose. The master dose was prepared at the target live cell concentration for further DfA container closure fill. One adjustment of the live cell concentration to within the allowable range may be performed. A calculated volume of the bulk dose was transferred to a new tube and re-suspended in a designated volume of vehicle solution based on the bulk dose live cell concentration result. This step formulated the target master dose based on the intended clinical dose level. The master dose was then sampled for total and live cell concentration to enable calculation of percent viability. If the enumeration did not meet the assay validity criteria, one re-sampling was performed. If the repeated master dose enumeration failed, the run was terminated. When a valid result was obtained, if the live cell concentration was not within the allowable range for the intended clinical dose, the master dose concentration was adjusted. If the viability did not meet the specification, the run was terminated. If both criteria were met, the adjusted master dose was filled into the DfA container closure.

The master dose was adjusted to within the allowable live cell concentration range. If the master dose needed to be diluted to a lower live cell concentration, vehicle solution was added to the master dose and mixed. If the master dose needed to be concentrated, the cells were pelleted via centrifugation and a calculated volume of supernatant was removed from the dose. The adjusted master dose was then mixed to resuspend the cells and sampled for total and live cell concentration to enable calculation of percent viability. If the enumeration did not meet the assay validity criteria, one re-sampling was performed. If the repeated adjusted master dose enumeration failed, the run was terminated. When a valid result was obtained, if the live cell concentration was not within the allowable range for the intended clinical dose or if the viability did not meet the specification, the run was terminated. If both criteria were met, the adjusted master dose was filled into the DfA container closure.

The time at which the final master dose was prepared was recorded, and the expiry time and date was calculated from this value and recorded. The final master dose was used to fill two doses of 50 μL each (1 clinical dose and 1 back-up dose) into pre-inspected tubes after uniformly resuspending the master dose by gentle pipetting. Additionally six 1.5 mL aliquots of vehicle solution were filled into pre-inspected tubes. The final live cell concentration and percent viability determined from enumeration of the master dose were recorded for release of the DfA. Additional samples of the master dose and the vehicle solution were taken for sterility and endotoxin testing. Appearance was evaluated by visual inspection of the final DfA and vehicle solution in their respective container closure systems. Interim release of the DfA for patient administration was based upon appearance, viability, live cell concentration, and endotoxin testing whereas interim release of vehicle solution for patient administration was based upon appearance and endotoxin testing. Primary container labels were applied across the top of each respective DfA and vehicle solution tube such that the ends of the labels adhere to the rack the tube is contained in, and each rack containing tubes was packaged in a sterile sampling bag and maintained at 1-8° C. The packaged racks, DfA and vehicle solution tubes were transferred to overnight storage until interim QC testing and interim release of the DfA and vehicle solution were completed. After overnight storage, DfA and vehicle solution packages were transferred to a temperature-controlled package which was monitored during transportation to the surgical administration site.

### Example 2: Aggregate Analysis Assay of Cell Product Comprising Cell Aggregates

Aggregate presence and quantity are important for downstream clinical use. The drug product (DP) was cryopreserved as multicellular aggregates (clusters) on the final day of the manufacturing process. The aggregate analysis test was used to confirm the presence and quantity of cell aggregates in the DP post-thaw.

The aggregate analysis assay was performed to characterize the size of aggregates in a cell product comprising cell aggregates as well as the fraction of cells present as aggregates. Briefly, a vial of cryopreserved cell product comprising cell aggregates at a concentration of 25×10⁶ cells per vial in 1 mL CryoSTOR® CS10 solution was thawed and mixed by gently inverting 2 to 3 times. A syringe containing 1 mL Balanced Salt Solution supplemented with 0.2% human serum albumin, hereafter BSS+HSA, was attached to the vial using an AT-Adapt™ Vented Vial Access Device. The BSS+HSA solution was injected into the vial at a rate of approximately 3 to 5 drops per second. The suspension of cell product comprising cell aggregates was then drawn into the syringe using and subsequently transferred to a 50 mL conical tube at a rate of approximately 3 to 5 drops per second. An additional 6 mL of BSS+HSA was added to the aggregate suspension in the 50 mL conical tube at a rate of 3-5 drops per second. The vial was washed with 2 mL of BSS+HSA to ensure all cells were collected, and the wash was added to the 50 mL conical tube.

The aggregate suspension was then centrifuged at 50×g for 5 minutes at room temperature. All but approximately 1 mL of supernatant was removed without disturbing the pellet. Next, 5 mL of BSS+HSA was added to dislodge and partially resuspend the pellet. The pellet and supernatant were collected and transferred to a 15 mL conical tube using a 10 mL serological pipet. The 50 mL conical tube was then washed with 5 mL BSS+HSA to ensure all cells were collected, and the wash was transferred to the 15 mL conical tube containing the aggregate suspension. The aggregate suspension was centrifuged at 50×g for 5 minutes at room temperature. All but approximately 500 μL was removed without disturbing the pellet. Next, 10 mL of BSS+HSA was added with care taken not to dislodge the pellet. The cells were centrifuged again at 50×g for 5 minutes at room temperature. All but approximately 100 μL was removed without disturbing the pellet. The pellet was then loosened by tapping and resuspended in 75 μL BSS+HSA by triturating 3-5 times. Next, 10 μL of aggregate suspension was aliquoted into 1.5 mL Eppendorf tubes in triplicate for particle size and percentage aggregate analysis. Additionally, 5 μL of aggregate suspension was aliquoted into a 1.5 mL Eppendorf tube for imaging analysis.

A particle size analyzer with a 280 μm aperture tube was used to characterize the size of aggregates and the fraction of cells present as aggregates. The current was set to 1600 μA, and the gain was set to 2. Measurements were acquired for particle diameter size (measured in μm) and aggregate percentage (measured by aggregate volume divided by total cell volume, which includes single cells) for each 10 μL sample (FIG. 1).

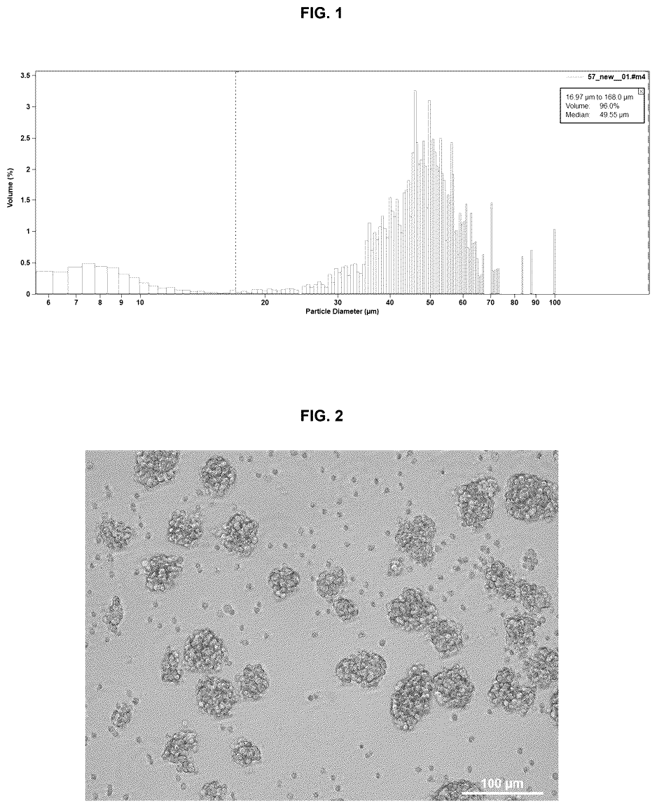

A DMi1 inverted microscope was used to image the remaining 5 μL sample of aggregate suspension. Briefly, the 5 μL sample and 150 μL of BSS+HSA were mixed and subsequently imaged with 20× magnification. At least 3 images in different fields of view were taken. A representative image is shown in FIG. 2.

Next, a particle size analyzer was used to measure the volume of cell clusters distributed across a specific diameter range and the volume of single-cell distribution for each lot across multiple samples. The method detected aggregate diameters between about 16.97 μm to about 168 μm as cellular aggregates. The ratio of aggregates to total cells was then calculated and reported as percentage biomass. The specification of ≥70% biomass was initially set by using historical lots (data not shown). Given that lower limit (mean minus two standard deviations; “mean−2SD” hereafter) was 0.8 (80%) and the minimum value of the data set was 0.7 (70%), ≥70% (0.7) was determined to be the percentage biomass specification.

This specification was confirmed using ten lots of DP (Table 5) and supported by results from lots used in pre-clinical studies, all of which met the specification of ≥70%. In addition to percentage biomass, median aggregate diameter (D₅₀) values were measured per batch.

TABLE 5
Summary of Statistical Analysis for Aggregate Analysis

| Parameter | Statistical Value |
|---|---|
| Number of lots | 10 |
| Mean (weighted) | 90% |
| SD (weighted) | 3 |
| Mean − 2SD (weighted) | 84% |

### Example 3: Vimentin Gene Expression by ddPCR of cDNA from Cell Product Comprising Cell Aggregates

Vimentin (VIM) is an intermediate filament protein responsible for maintaining cell shape and cytoplasm integrity as well as stabilizing cytoskeletal interactions. Within the retina, VIM is primarily expressed in retinal glia (Muller glia and retinal astrocytes) and retinal progenitor cells. VIM serves as an off-target marker for glial cells and for retinal progenitor cells that have the potential to become glial cells. VIM expression level is important for measuring the integrity of the DP for downstream clinical uses.

VIM gene expression was assessed by droplet digital PCR (ddPCR) for cDNA derived from a cell product comprising cell aggregates. Briefly, sample cDNA at a concentration of 25 ng/μL was diluted to 5 ng/μL using PCR certified water. A positive control of cDNA isolated from hepatocytes was diluted to 1 ng/μL using PCR certified water. The sample and positive control dilutions were vortexed to mix and spun down. Each sample was processed in triplicate along with a negative control group containing PCR Certified Water only.

A PCR master mix was prepared as shown in Table 6. The master mix was vortexed to mix and spun down. Then, the master mix was transferred to a 96-well plate with 20 μL per well.

TABLE 6
Master Mix per 20 μL Reaction.

| Component | Volume per Reaction |
|---|---|
| 2x ddPCR probes (no dUTP) Supermix | 12.5 μL |
| PCR Certified Water | 5.0 μL |
| 20x VIM-FAM Assay | 1.25 μL |
| 20x SDHA-VIC Assay | 1.25 μL |

For each well, 5 μL of sample cDNA, positive control cDNA (from hepatocytes), or negative control (PCR Certified Water) was transferred to each well containing 20 μL master mix. The plate was sealed at 180° C. for 5 seconds. The plate was then vortexed at each corner for at least 15 seconds each. Then plate was spun down for 30 seconds to minimize air bubbles.

Droplets were generated according to the manufacturer's instructions. The plate was sealed again at 180° C. for 5 seconds. PCR was then performed to produce amplified droplets using the parameters in Table 7 with a volume setting of 40 μL and a lid temperature setting of 105° C.

TABLE 7
Thermocycling Conditions for ddPCR.

| | | | |
|---|---|---|---|
| Temperature | Time | Cycles | Ramp Rate |

| | | | | |
|---|---|---|---|---|
| 95° C. | 10 | minutes | 1 | 2° C./second |
| 94° C. | 30 | seconds | 40 | 2° C./second |
| 57° C. | 90 | seconds | | 2° C./second |
| 98° C. | 10 | minutes | 1 | 2° C./second |
| 4° C. | 30 | minutes | 1 | — |

| | | | |
|---|---|---|---|
| 4° C. | ∞ | ∞ | — |

The amplified droplets for each sample were assessed according to manufacturer's instructions to assess VIM in channel 1 and SDHA (reference gene) in channel 2 with FAM/VIC probes. Copies per μL of VIM divided by copies per μL of SDHA were reported as a ratio (copies/copy). For a cell product to be suitable for downstream applications, acceptable samples must contain at least 10,000 droplets and have a percentage coefficient of variation of less than or equal to 25%. The negative control (PCR Certified Water) must have at least two replicates with less than or equal to 0 copies/copy. Further, the positive control cDNA (from hepatocytes) must have between 100-500 SDHA copies per μL. The cDNA derived from a cell product comprising cell aggregates must contain between 800-4,000 SDHA copies per μL. A cell product suitable for downstream applications will contain no more 0.50 copies/copy (copies per μL of VIM divided by copies per μL of SDHA). Outliers were assessed and removed, if appropriate, with a Dixon Q Test.

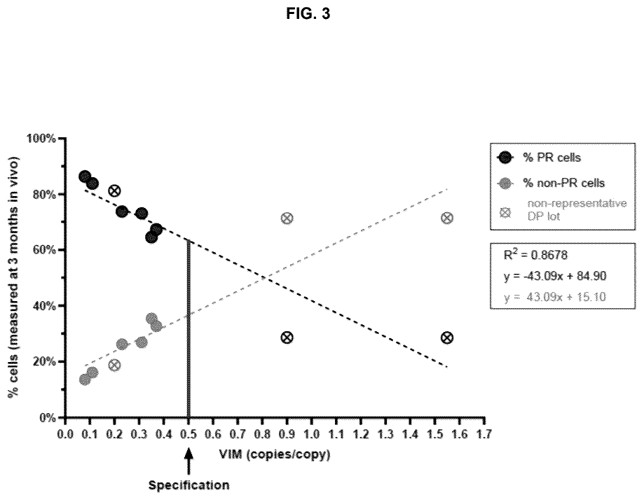

Pre-clinical studies indicated that higher VIM expression levels in DP was associated with an increased presence of non-photoreceptor cells (non-PRs). The inverse relationship between VIM expression levels and photoreceptor (PR) presence highlighted the need for careful control of VIM expression (FIG. 3). FIG. 3 shows non-clinical data generated using three non-representative DP batches which have high (e.g., out of proposed specification for clinical lots) VIM expression.

These data demonstrate negative outcomes with high VIM expression, including (1) the majority of engrafted human cells were non-PR cells in vivo at 3 months and (2) qualitatively, non-PR cells were identified as glial cells in vivo.

Given the pre-clinical findings with these non-representative, high V/M-expressing DP batches, potential clinical implications were anticipated with high VIM expression levels in DP. The excess of non-PR cells associated with elevated VIM expression could potentially compromise the therapeutic outcome or pose a safety concern. Since glial cells have proliferation potential, high VIM expression in DP may disrupt the target cellular composition. The potential clinical outcomes derived from the buildup of non-PRs emphasized the importance of controlling VIM expression levels to maintain the therapeutic integrity of the DP.

The VIM expression specification was established using 13 lots of DP with 80 data points in total (Table 8). The statistical analysis (mean+2SD in mixed model) performed showed that the upper limit was 0.5 copies/copy, therefore the specification was set as s 0.5 copies/copy. The proposed specification was supported by the current DP manufacturability and by the fact the lots with atypical composition/performance in pre-clinical studies would fall outside of the specification.

Taken together, setting a VIM specification of 50.5 copies V/M/copy SDHA safeguarded against an overabundance of off-target cells such as retinal glia and retinal progenitor cells supporting the primary therapeutic function of the photoreceptor cells in the DP and enhanced patient safety.

TABLE 8
Summary of Statistical Analysis for VIM gene expression (ddPCR)

| Parameter | Statistical Value |
|---|---|
| Number of DP lots | 13 |
| Number of Observations | 80 |
| Mean | 0.3 VIM/copy SDHA |
| SD | 0.1 VIM/copy SDHA |
| Mixed Model Intercept | 0.3 VIM/copy SDHA |
| Estimate | |
| Mixed Model Total Standard | 0.1 VIM/copy SDHA |
| Deviation | |
| Mean + 2SD | 0.5 VIM/copy SDHA |

### Example 4: Percentage Viability of a Cell Product Comprising Cell Aggregates

A viability assessment was performed for a cell product comprising cell aggregates. Briefly, a vial of cryopreserved cell product comprising cell aggregates at a concentration of 25×10⁶ cells per vial in 1 mL CryoSTOR® CS10 solution was thawed and mixed by gently inverting 2 to 3 times. A syringe containing 1 mL BSS+HSA solution was attached to the vial using an AT-Adapt™ Vented Vial Access Device. The BSS+HSA solution was injected into the vial at a rate of approximately 3 to 5 drops per second. The suspension of cell product comprising cell aggregates was then drawn into the syringe using and subsequently transferred to a 15 mL conical tube at a rate of approximately 3 to 5 drops per second. Next, an additional 6 mL of BSS+HSA was added to the aggregate suspension in the 15 mL conical tube at a rate of 3-5 drops per second. The vial was washed with 2 mL of BSS+HSA to ensure all cells were collected, and the wash was added to the 15 mL conical tube.

The aggregate suspension was then centrifuged at 50×g for 5 minutes at room temperature. All but approximately 500 μL of supernatant was removed without disturbing the pellet. Next, 5 mL of BSS+HSA was added to dislodge and partially resuspend the pellet. The pellet and supernatant were collected and transferred to a new 15 mL conical tube containing 400 μL of BSS+HSA using a 5 mL serological pipet. The old 15 mL conical tube was then washed with 5 mL BSS+HSA to ensure all cells were collected, and the wash was transferred to the new 15 mL conical tube containing the aggregate suspension. The aggregate suspension was centrifuged at 50×g for 5 minutes at room temperature. All but approximately 400 μL was removed without disturbing the pellet.

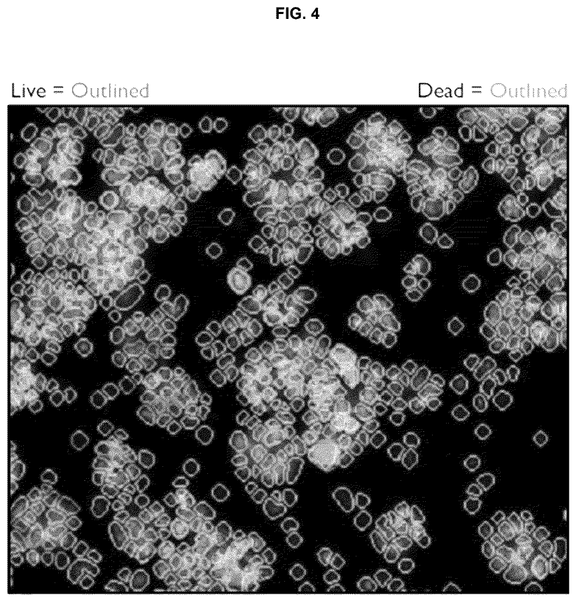

A two-fold dilution series of the aggregate suspension was prepared at 1:10, 1:20, 1:40, 1:80, and 1:160 with BSS+HSA. Cells were mixed by trituration approximately 3 to 5 times prior to addition to next dilution tube. Then, 1.5 μL of acridine orange/propidium iodine (AOPI) staining solution was added to each dilution tube. Samples were triturated three times and subsequently plated in a 96-well plate. Cells were imaged for AO and PI stain at 10× magnification within 10 minutes of plating. Representative images are shown in FIG. 4. Percentage viability of the aggregate suspension may be calculated as (1−(number of PI-positive cells/(number of AO-positive cells−number of PI-positive cells)))×100. Percentage viability of the aggregate suspension may also be calculated as (number of AO-positive cells/(number of AO-positive cells+number of PI-positive cells)×100.

### Example 5: Measurement of Percentage Viability of Cell Product Comprising Cell Aggregates Using a Hybrid Counting Method

A second viability assessment was performed for a cell product comprising cell aggregates. Briefly, a vial of cryopreserved cell product comprising cell aggregates at a concentration of 25×10⁶ cells per vial in 1 mL CryoSTOR® CS10 solution was thawed and mixed by gently inverting 2 to 3 times. A syringe containing 1 mL BSS+HSA solution was attached to the vial using an AT-Adapt™ Vented Vial Access Device. The BSS+HSA solution was injected into the vial at a rate of approximately 3 to 5 drops per second. The suspension of cell product comprising cell aggregates was then drawn into the syringe using and subsequently transferred to a 50 mL conical tube at a rate of approximately 3 to 5 drops per second. Next, an additional 6 mL of BSS+HSA was added to the aggregate suspension in the 50 mL conical tube at a rate of 3-5 drops per second. The vial was washed with 2 mL of BSS+HSA to ensure all cells were collected, and the wash was added to the 50 mL conical tube.

The aggregate suspension was then centrifuged at 50×g for 5 minutes at room temperature. All but approximately 500 μL to 2000 μL of supernatant was removed without disturbing the pellet. Next, 5 mL of BSS+HSA was added to dislodge and partially resuspend the pellet. The pellet and supernatant were collected and transferred to a 15 mL conical tube containing 400 μL of BSS+HSA using a 5 mL serological pipet. The 50 mL conical tube was then washed with 5 mL BSS+HSA to ensure all cells were collected, and the wash was transferred to the 15 mL conical tube containing the aggregate suspension. The aggregate suspension was centrifuged at 50×g for 5 minutes at room temperature. All but approximately 500 μL was removed without disturbing the pellet. The aggregate suspension was again centrifuged at 50×g for 5 minutes at room temperature. All but approximately 400 μL was removed, and the cells were resuspended by gently triturating approximately 5 times.

Total cell counts were determined by combining 30 μL of aggregated cell suspension and 470 μL of Solution 10™. The resulting mixture was triturated approximately 2 to 3 times with a pipette, and samples were then incubated for up to 5 minutes at room temperature. Subsequently, samples were triturated up to 30±2 times using a P1000 pipette set to 400 μL with force to isolate the nuclei. Samples were then transferred to a plate (3 replicates, 50 μL samples), and 50 μL of AOPI was mixed with the cells. Total cell counts (PI positive cells) were measured within two hours. Brightfield and fluorescence collection modes were used to count total cells across two sub-channels, with cell diameter for Channel 1 set between 3.0 to 25.0 μm and cell diameter for Channel 2 set between 3.0 and 20.0 μm.

Live cell counts were also determined from the same starting cell composition. Briefly, 30 μL of aggregated cell suspension was combined with 400 μL 10× TrypLE™. Next, the cells were incubated for 35±5 minutes at 37° C. Subsequently, 770 μL of pre-warmed (to 37° C.) quench reagent (DMEM/F12+B27+DNase) was added, and each sample was triturated up to 20±2 times with a P1000 pipette set to 770 μL. Samples were then transferred to a plate (3 replicates, 50 μL samples), and 50 μL of AOPI was mixed with the cells. Live cell counts (AO positive cells) were measured within two hours. Brightfield and fluorescence collection modes were used to count live cells across two sub-channels, with cell diameter for Channel 1 set between 3.0 to 25.0 μm and cell diameter for Channel 2 set between 3.0 and 20.0 μm.

### Drug Product (DP)

A statistical analysis was performed on 40 data points obtained from 7 DP lots for viability using this method (Table 9). Despite the statistical outcome from mean−2SD (36.83%), the specification was set at ≥50%, considering regulatory expectation of viability at 70% and inherent method limitation for the current viability method. While the industry standard typically leans towards a specification of ≥70%, the specification of ≥50% was chosen as a starting point. Notably, the latest batches have shown the highest observed viability rates, ranging from 58% to 62%, reflecting ongoing process improvements.

Under the initial feasibility studies of the method in the previous Example, images were taken from diluted cell aggregates that were stained with a live/dead dye (AO/PI), without the need for dissociation. This method minimized cell manipulation and preserved the integrity of the aggregates, potentially providing a more representative assessment of cell viability in the minimally manipulated sample. In a side-by-side comparison of the two methods across 9 research lots, the other method consistently showed higher viability, approximately 15 to 20% higher than the results obtained from the current method.

TABLE 9
Summary of Statistical Analysis for Viability

| Parameter | Statistical Value |
|---|---|
| Number of DP lots | 7 |
| Number of Observations | 40 |
| Mean | 55% |
| SD | 9.6% |
| Mixed Model Intercept Estimate | 56% |
| Mixed Model Total Standard Deviation | 9.5% |
| Mean − 2SD | 36.83% |

### Dose for Administration (DfA)

Dose for Administration (DfA) cell viability was also measured to quantify the percentage of live cells to support product quality. This process involved two independent cell counting assays: one for determining the live cell concentration and another for the total cell concentration. From these measurements, the percentage of viable cells (also referred to as viability) was calculated. To determine live cell concentration, cell aggregates were dissociated both enzymatically and manually using the reagent 10× TrypLE™ Select and trituration. The enzymatic reaction was then quenched, and live cells were quantified using the fluorescence dye acridine orange/propidium iodide (AO/PI). For total cell concentration, cells were lysed with Solution 10™ lysis buffer, and total nuclei were quantified using the same fluorescent dye, AO/PI. Both live and total cell counts were performed. The method was qualified and confirmed to be fit for purpose.

A statistical analysis using tolerance interval of DfA viability was performed on representative DfA lots for 1×10⁶ dose (n=15), 2×10⁶ dose (n=8), 3×10⁶ dose (n=8), and 4×10⁶ dose (n=16). A summary of results is presented in Table 10. A specification of ≥40% viability was proposed based on four factors described below: 1) the DP entering the DfA process was at the viability specification of ≥50%. Considering additional cell manipulation steps during the DfA process, such as thawing and aggregate enrichment, a potential decrease in viability was accounted for; 2) the current DfA manufacturability and statistical analysis were considered. The statistical analysis conducted reinforced the proposed specification. Tolerance interval lower limits for clinical doses 1×10⁶ dose (n=15), 2×10⁶ dose (n=8), and 3×10⁶ dose (n=8) fell within the 40% viability range. For 4×10⁶ dose, the tolerance interval lower limit was 34%. However, all n=16 lots of 4×10⁶ dose passed the specification of 40%. Processing the DP to DfA demonstrated a data set that statistically fit within a tolerance interval of >40%, confirming manufacturability. The specification was further supported by the DP lot used for the preclinical study lot, which was used to generate DfA with a viability of 43% and 42%. Although the DfA were not evaluated preclinically, this viability served as a benchmark for viability output of the representative DfA manufacturing process; 3) the potential drop in viability post-device was evaluated, considering patient safety and efficacy. The average viability decrease from pre-device to post-device administration was −2.9% for the 1×10⁶ dose, −6.6% for the 2×10⁶ dose, −5.7% for the 3×10⁶ dose, and −5.9% for the 4×10⁶ dose. An in-use stability study supported the specification. Preliminary in-use stability data indicated viability levels >40% from T=0 hours to T=28 hours, ensuring viability remained within acceptable limits from DfA manufacture to patient administration; and 4) correlation with the orthogonal imaging assay was also considered.

In conclusion, the viability specification of ≥40% was justified based on manufacturability, process impacts, and statistical analysis. Additionally, patient safety, in-use stability studies, and correlation with orthogonal imaging assays have been considered. This specification ensures a balance between maintaining cell viability and feasibility for clinical applications.

TABLE 10
Summary of Statistical Analysis for Viability
Parameter Statistical Value

| Clinical Dose | 1 × 10⁶ | 2 × 10⁶ | 3 × 10⁶ | 4 × 10⁶ |
|---|---|---|---|---|
| Number of Lots | 15 | 8 | 8 | 16 |
| Mean | 56 | 58 | 55 | 54 |
| Standard Deviation | 6.0 | 5.9 | 4.3 | 7.5 |
| Tolerance Interval | 40 | 42 | 43 | 34 |
| 95/92.4 Lower Limit | | | | |

| | |
|---|---|
| Specification | ≥40% |

### Example 6: Flow Cytometric Analysis of Cell Product Comprising Cell Aggregates

Using the methods of Example 1, PRP markers were used to evaluate the purity of the cells. AIPL1, RCVRN, PAX6, CHX10, TYRP1, Ki67, PPP4R4, and NR2E3+ are all suitable markers for confirming purity of a composition comprising PRPs. AIPL1, RCVRN, PPP4R4, and NR2E3 are on-target markers. Expression of these markers indicates rod and cone photoreceptor development. PAX6, CHX10, TYRP1, and Ki67 are off-target markers. Low expression of these markers indicates a higher PRP cell purity. PRPs expressing on-target cell purity markers and lacking off-target cell impurities are deemed critical for DP quality and any downstream applications. Therefore, each marker was measured using flow cytometry and is further described in the following subsections.

Briefly, to characterize the cell product comprising cell aggregates, cells were dissociated, fixed, and stained using the following primary antibodies: anti-AIPL1 (1:1,000), anti-RCVRN (1:10,000), anti-PAX6 (1:2,000), anti-TYRP1 (1:1,000), anti-CHX10 Alexa Flour 647 1:1,000), anti-Ki67 AF647 (1:50), anti-NR2E3 (1:800), and/or anti-PPP4R4 (1:3,500). For unconjugated primary antibodies, the following secondary antibodies were used for detection: donkey anti-rabbit IgG Alexa Fluor 488 (1:1,000), goat anti-mouse IgG2a AF488 (1:2,000), and/or donkey anti-rabbit IgG AF647 (1:4,000).

Cells were washed twice with Perm Buffer and stained with primary antibodies overnight at 4° C., protected from light. Stained cells were then washed twice with Perm Buffer and stained with secondary antibodies for between 60 minutes and 120 minutes at room temperature, protected from light. Cells were washed twice with Stain Buffer and then analyzed using flow cytometer, acquiring at least 10,000 events per sample.

### AIPL1 Marker for Flow Cytometry

Aryl hydrocarbon receptor interacting protein like 1 (AIPL1) is an on-target pan PRP specific marker which ensures commitment to the PRP phenotype. Its expression is detected early in developing rod and cone photoreceptors (Kolandaivelu et. al., 2014) and is highly specific (van der Spuy et. al., 2003).

The specification of ≥90% for AIPL1+ cells was initially set using historical lots. While the lower limit was 94%, according to the statistical method of mean−2SD, and the minimum value of the data set used to calculate this limit was 93%, the specification for AIPL1 was maintained at ≥90% because at this phase, it was deemed appropriate.

This specification was confirmed using 9 lots of DP. Table 11 provides a summary of the statistical analysis for AIPL1, as well as results from lots used in pre-clinical studies, all of which met this ≥90% specification.

TABLE 11
Summary of Statistical Analysis for AIPL1 (Flow Cytometry)

| Parameter | Statistical Value |
|---|---|
| Number of lots | 9 |
| Mean (weighted) | 96% |
| SD (weighted) | 0.9 |
| Mean − 2SD (weighted) | 94% |

### RCVRN Marker for Flow Cytometry

Recoverin (RCVRN) is an on-target pan PRP marker. RCVRN is a common marker used to identify rod and cone photoreceptors (Zang et. al., 2018) and thereby provides confirmation of product identity. RCVRN is upregulated during photoreceptor development and its expression persists in outer segments of mature photoreceptors and a subset of bipolar cells (Gunhan et. al., 2003).

The specification of ≥90% for RCVRN+ was initially set using historical lots (data not shown). While the lower limit was 92%, according to statistical method of mean−2SD, the specification for RCVRN was set at ≥90%, since the minimum value of the data set used to calculate this limit was 90%.

The specification was confirmed using 9 lots of DP. Table 12 provides a summary of the statistical analysis for RCVRN, as well as results from lots used in pre-clinical studies, all of which met this ≥90% specification.

TABLE 12
Summary of Statistical Analysis for RCVRN (Flow Cytometry)

| Parameter | Statistical Value |
|---|---|
| Number of lots | 9 |
| Mean (weighted) | 97% |
| SD (weighted) | 2.1 |
| Mean − 2SD (weighted) | 92% |

### PAX6 Marker for Flow Cytometry

Paired box protein 6 (PAX6) is an off-target marker that detects neural retinal progenitor cells, ganglion cells, amacrine, and Muller glial cells, which are potential impurity populations. Therefore, PAX6 should be controlled to maintain product quality. PAX6 transcription factor determines cell commitment to the eye field, specifically driving pluripotent cells towards multipotent retinal progenitor cell or retinal pigment epithelium populations (Heavner et. al., 2012; Marquardt et. al., 2001). PAX6 expression is maintained throughout retinal development in retinal progenitor cells and persists in terminally differentiated amacrine cells and retinal ganglion cells, as well as a subset of Muller glia (Hitchcock et. al., 1996; Karl et. al., 2008; Lalitha et. al., 2020; Roesch et. al., 2008).

The specification of 57% for PAX6+ cells was initially set by historical lots (data not shown). The upper limit was 2% according to the statistical method of mean+2SD, and the maximum value of the data set was 4%. The specification was maintained at ≤7%.

The specification was confirmed using 9 lots of DP. Table 13 provides a summary of the statistical analysis for PAX6, as well as results from lots used in pre-clinical studies, all of which met this ≤7% specification.

TABLE 13
Summary of Statistical Analysis for PAX6 (Flow Cytometry)

| Parameter | Statistical Value |
|---|---|
| Number of lots | 9 |
| Mean (weighted) | 2% |
| SD (weighted) | 0.4 |
| Mean + 2SD (weighted) | 2% |

### CHX10 Marker for Flow Cytometry

CHX10 homeodomain-containing homolog (CHX10), also known as VSX2 (visual system homeobox 2), is an off-target marker that detects post-mitotic bipolar cells, neural retinal progenitor cells, and Muller glia cells, which are potential impurity populations. Therefore, CHX10 should be controlled to maintain product quality. CHX10 is the earliest known transcription factor expressed by and required for developing retinal neurons, confirmed through studies using induced pluripotent stem cells (iPSCs) from mice and humans (Zou et. al., 2012). CHX10 is also a key determinant of retinal bipolar cell differentiation (Livne-Bar et. al., 2006) and is expressed by a subset of Muller glia (Rowan et. al., 2004). CHX10 staining identified the proportion of neural retinal progenitor cells and bipolar cells within the DP.

The specification of ≤7% CHX10+ cells was initially set by using historical lots (data not shown). While the upper limit was 2% according to the statistical method of mean+2SD, and the maximum value of the data set was 3%, the specification was maintained at 57%, which was deemed appropriate at this phase.

The specification was confirmed using 9 lots of DP. Table 14 provides a summary of the statistical analysis for CHX10, as well as results from lots used in pre-clinical studies, all of which met this ≤7% specification.

TABLE 14
Summary of Statistical Analysis for CHX10 (Flow Cytometry)

| Parameter | Statistical Value |
|---|---|
| Number of lots | 9 |
| Mean (weighted) | 1% |
| SD (weighted) | 0.4 |
| Mean + 2SD (weighted) | 2% |

### TYRP1 Marker for Flow Cytometry

Tyrosinase related protein 1 (TYRP1) is an off-target marker that detects developing and mature retinal pigment epithelium (Qiu et. al., 2016), which is a potential impurity population. Therefore, TYRP1 should be controlled to maintain product quality.

The specification of 50.5% TYRP1+ cells was initially set by using historical lots (data not shown). While the upper limit (mean+2SD) was 0.031% and the maximum value of the data set was 0.05%, the specification was maintained at 50.5%, as it is deemed phase appropriate, and the data set may be near the quantitation limit of the assay.

The specification was confirmed using 9 lots of DP. Table 15 provides a summary of the statistical analysis for TYRP1, as well as results from lots used in pre-clinical studies, all of which met this 50.5% specification.

TABLE 15
Summary of Statistical Analysis for TYRP1 (Flow Cytometry)

| Parameter | Statistical Value |
|---|---|
| Number of lots | 9 |
| Mean (weighted) | 0.01% |
| SD (weighted) | 0.010 |
| Mean + 2SD (weighted) | 0.031% |

### Ki67 Marker for Flow Cytometry

Ki67 is an off-target marker that detects proliferating cells in all active phases of the cell cycle (Qiu et. al., 2016), and therefore, needs to be controlled to maintain low impurity profile for overall product quality.

The specification of 50.5% Ki67+ cells was initially set by using historical lots (data not shown). While the upper limit (mean+2SD) is 0.2% and the maximum value of the data set of 0.3%, the specification was maintained at ≤0.5%, as it was appropriate at this phase.

The specification was confirmed using 9 lots of DP. Table 16 provides a summary of the statistical analysis for Ki67, as well as results from lots used in pre-clinical studies, all of which met this ≤0.5% specification.

TABLE 16
Summary of Statistical Analysis for Ki67 (Flow Cytometry)

| Parameter | Statistical Value |
|---|---|
| Number of lots | 9 |
| Mean (weighted) | 0.1% |
| SD (weighted) | 0.07 |
| Mean + 2SD (weighted) | 0.2% |

### PPP4R4 Marker for Flow Cytometry

Protein phosphatase 4 regulatory subunit 4 (PPP4R4) is an on-target marker that identifies cone-specific PRPs and is considered relevant for product efficacy. It is a cytoplasmic regulatory protein expressed in cone photoreceptors in the retina. Cones are the “active” population for visual acuity including central and color vision, and their desired frequency is calculated based on the lowest dose and predicted survival rate (1×10⁶ total viable cells).

The specification of ≥20% and ≤60% PPP4R4+ cells was set using 14 lots of DP with 49 data points in total. The statistical analysis (mean±2SD) performed showed that the lower limit was 22% and the upper limit was 54%, thereby the specification was set at ≥20% and ≤60%.

The specification was confirmed using 14 lots of DP. Table 17 provides a summary of the statistical analysis for PPP4R4, as well as results from lots used in pre-clinical studies, all of which met this ≥20% and ≤60% specification.

TABLE 17
Summary of Statistical Analysis for PPP4R4 (Flow Cytometry)

| Parameter | Statistical Value |
|---|---|
| Number of DP lots | 14 |
| Number of Observations | 49 |
| Mean | 38% |
| SD | 8.5% |
| Mixed Model Intercept Estimate | 3.529% |
| Mixed Model Total Standard Deviation | 0.229% |
| Mean + 2SD | 22-54% |

### NR2E3 Marker for Flow Cytometry

Nuclear receptor subfamily 2 group E member 3 (NR2E3) is an on-target marker that identifies rod-specific photoreceptor precursor cells. It belongs to the large family of nuclear receptor transcription factors that promotes and maintains the function of rod photoreceptors, which are important for cell health and for low light vision. The specifications for PRPs (AIPL1≥90%, RCVRN≥90%) and cones (PPP4R4≥20% and ≤60%) indirectly ensured that there was an appropriate population of rods present in DP.

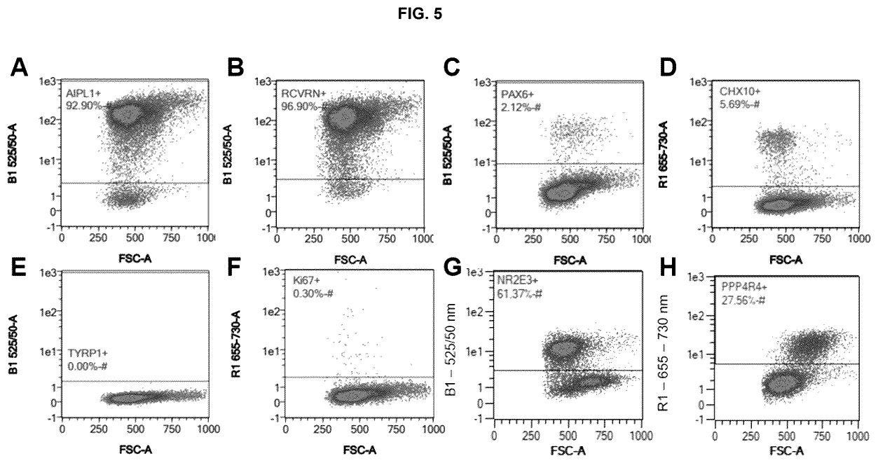

Cells which are suitable for downstream applications displayed at least 90% AIPL1+ cells (FIG. 5A), at least 90% RCVRN+ cells (FIG. 5B), less than or equal to 7% PAX6+ cells (FIG. 5C), less than or equal to 7% CHX10+ cells (FIG. 5D), less than or equal to 0.5% TYRP1+ cells (FIG. 5E), less than or equal to 0.5% Ki67+ cells (FIG. 5F), NR2E3+ cells (FIG. 5G), and between 20% and 60% PPP4R4+ cells (FIG. 5H).

### Example 7: Stability of Dose for Administration

The stability of a cell product comprising cell aggregates formulated as a Dose for Administration (DfA) was assessed by measuring viable cell concentration, percent cell viability, and percent aggregate biomass after a 1-8° C. hold period. Briefly, a master dose was created at Time 0 (To) for two clinical dose targets, 1×10⁶ and 4×10⁶ viable cells, for four replicates. Samples of the master dose were taken to assess viable cell concentration (60 μL), total cell concentration (15 μL) and percentage aggregate biomass (50 μL) at T₀. Next, fifteen DfAs were aliquoted in 50 μL volumes from the master dose and held at 1-8° C. for 4-7 hours, 24-27 hours, or 28-31 hours. At each timepoint, two doses were assessed for percentage aggregate biomass and three doses were assessed for percentage viability and viable cell concentration. The results are shown in Table 18 (viable cell concentration), Table 19 (percentage viability), and Table 20 (percentage aggregate biomass).

TABLE 18
Viable Cell Concentration After 1-8° C. Hold

| Clinical | | | Viable Cell | | Total Cell | |
|---|---|---|---|---|---|---|
| Dose | | | Concentration | | Concentration | |
| Target | | | | Standard | | Standard |
| (viable | Sampling | | Mean | Deviation | Mean | Deviation |
| cells) | Point | | (cells/mL) | (cells/mL) | (cells/mL) | (cells/mL) |
| 1 × 10⁶ | T₀ | | 3.22 × 10⁷ | 3.0 × 10⁶ | 5.96 × 10⁷ | 8.8 × 10⁶ |
| | 4-7 | hours | 2.93 × 10⁷ | 6.7 × 10⁶ | 5.91 × 10⁷ | 9.3 × 10⁶ |
| | 24-27 | hours | 2.87 × 10⁷ | 5.4 × 10⁶ | 5.81 × 10⁷ | 7.2 × 10⁶ |
| | 28-31 | hours | 2.86 × 10⁷ | 4.5 × 10⁶ | 6.07 × 10⁷ | 8.2 × 10⁶ |
| 4 × 10⁶ | T₀ | | 1.21 × 10⁸ | 9.0 × 10⁶ | 2.14 × 10⁸ | 2.1 × 10⁷ |
| | 4-7 | hours | 1.36 × 10⁸ | 2.1 × 10⁷ | 2.19 × 10⁸ | 1.8 × 10⁷ |
| | 24-27 | hours | 1.25 × 10⁸ | 3.1 × 10⁷ | 2.33 × 10⁸ | 2.4 × 10⁷ |
| | 28-31 | hours | 1.24 × 10⁸ | 2.6 × 10⁷ | 2.30 × 10⁸ | 2.8 × 10⁷ |

TABLE 19
Percentage Viability After 1-8° C. Hold

| | |
|---|---|
| Clinical | Percentage Viability |

| Dose Target | Sampling | | Standard |
|---|---|---|---|
| (viable cells) | Point | Mean | Deviation |
| 1 × 10⁶ | T₀ | 54.4% | 4.4% |

| | | | |
|---|---|---|---|
| 4-7 | hours | 49.7% | 7.8% |
| 24-27 | hours | 49.6% | 8.1% |
| 28-31 | hours | 47.2% | 5.3% |

| | | | |
|---|---|---|---|
| 4 × 10⁶ | T₀ | 57.1% | 7.6% |

| | | | |
|---|---|---|---|
| 4-7 | hours | 61.8% | 6.8% |
| 24-27 | hours | 53.0% | 8.3% |
| 28-31 | hours | 53.8% | 6.4% |

TABLE 20
Percentage Aggregate Biomass After 1-8° C. Hold

| | |
|---|---|
| Clinical | Aggregate Biomass |

| Dose Target | Sampling | | Standard |
|---|---|---|---|
| (viable cells) | Point | Mean | Deviation |
| 1 × 10⁶ | T₀ | 84.6% | 7.4% |

| | | | |
|---|---|---|---|
| 4-7 | hours | 80.7% | 8.0% |
| 24-27 | hours | 79.7% | 8.3% |
| 28-31 | hours | 79.6% | 8.5% |

| | | | |
|---|---|---|---|
| 4 × 10⁶ | T₀ | 91.0% | 2.8% |

| | | | |
|---|---|---|---|
| 4-7 | hours | 89.9% | 2.7% |
| 24-27 | hours | 88.9% | 2.4% |
| 28-31 | hours | 88.8% | 2.6% |

The results in Tables 18-20 demonstrate that the viable cell concentration, percentage viability, and percentage aggregate biomass of a DfA of a cell composition comprising cell aggregates remains stable after holding at 1-8° C. for 4-7 hours, 24-27 hours, and 28-31 hours after preparation of the master dose. In conclusion, the cell product comprising cell aggregates formulated as a DfA demonstrates an in-use stability of at least 28 hours from the master dose preparation.

### Example 8: Dose Assurance and Device Compatibility Assessment

The cell product comprising cell aggregates was assessed for the impact of passage through an exemplary delivery device (Constellation Vision System; Alcon #8065753048) on the viability of the cells. In addition, dose assurance under simulated use conditions was assessed to measure any cell loss that occurs during loading and delivery of the cell product comprising cell aggregates. Potential sources of cell loss include cell adhesion or loss during pipetting of the Dose for Administration (DfA), cell adhesion and loss within the DfA tube during loading of the DfA, loss to device dead volume, or decrease in viability from injection with the delivery system.

Briefly, DfAs were prepared from a master dose at four clinical dose targets (1×10⁶, 2×10⁶, 3×10⁶, and 4×10⁶ viable cells) and held at 0.5-8° C. for 21±3 hours prior to passage through the delivery device. A syringe (MicroDose Injection Kit; MedOne #3275) and cannula (PolyTip Funnel Cannula 25G/33G; MedOne, #3510) were primed with BSS+HSA vehicle solution, and then the DfAs were aspirated into the device via tubing (VFC Pak; Alcon #8065750957) with a maximum aspiration pressure setting of 350 mmHg. The DfAs were held within the delivery system in the vertical position for 2-8 minutes and then injected using the Constellation Vision System with a maximum pressure of 10 psi into a 10 μL volume of vehicle solution to mimic injection into the fluid environment of the eye.

After injection, the viable cell concentration and total cell concentration of the administered doses were measured to determine dose assurance and delivery system impact on cell viability for at least fourteen technical replicates. The resulting cell viability in the administered dose was compared to the corresponding master dose that was not passed through the delivery device. The viable cell concentration data are shown in Table 21 and the percentage viability relative to the master dose is shown in Table 22.

TABLE 21
Number of Viable Cells in Administered Dose

| | Administered Dose Viable Cell Number (cells) | | | |
|---|---|---|---|---|
| Clinical | | 95% CI of the Mean | | |
| Dose Target | | Lower | Upper | Standard |
| (viable cells) | Mean | Bound | Bound | Deviation |
| 1 × 10⁶ | 1.00 × 10⁶ | 9.44 × 10⁵ | 1.06 × 10⁶ | 1.0 × 10⁵ |
| 2 × 10⁶ | 1.81 × 10⁶ | 1.63 × 10⁶ | 1.99 × 10⁶ | 3.4 × 10⁵ |
| 3 × 10⁶ | 3.01 × 10⁶ | 2.86 × 10⁶ | 3.17 × 10⁶ | 2.9 × 10⁵ |
| 4 × 10⁶ | 3.94 × 10⁶ | 3.67 × 10⁶ | 4.21 × 10⁶ | 4.9 × 10⁵ |

TABLE 22
Viability Data of Administered Dose Relative to Master Dose

| Clinical | | | Viability Change from Master Dose to | | | |
|---|---|---|---|---|---|---|
| Dose | Administered Dose | | Administered Dose | | | |
| Target | Viability | | | 95% CI of the Mean | | |
| (viable | | Standard | | Lower | Upper | Standard |
| cells) | Mean | Deviation | Mean | Bound | Bound | Deviation |
| 1 × 10⁶ | 52.4% | 7.2% | −2.9% | −8.7% | 2.9% | 10% |
| 2 × 10⁶ | 51.9% | 7.9% | −6.6% | −10.0% | −3.1% | 7% |
| 3 × 10⁶ | 49.7% | 6.0% | −5.7% | −8.6% | −2.8% | 5% |
| 4 × 10⁶ | 47.6% | 5.1% | −5.9% | −8.3% | −3.5% | 4% |

The DfA preparation process and injection with the delivery system effectively administered doses with means of 1.00×10⁶, 1.81×10⁶, 3.01×10⁶, and 3.94×10⁶ viable cells for their respective clinical dose target, indicating minimal cell loss and thus accurate dosing. Furthermore, the average viabilities of the administered doses were 52.4%, 51.9%, 49.7% and 47.6% for each clinical dose targets, where the average change in viability between master dose and administered dose of −2.9%, −6.6%, −5.7%, and −5.9%, respectively. The results of this assessment demonstrate that the delivery system was capable of delivering the four clinical target doses with minimal impact to cell viability.

### Example 9: Impact of Administration with the Delivery System Under Simulated Worst-Case Scenario Conditions

To determine the compatibility of the cell product comprising cell aggregates with an exemplary delivery system under worst-case clinical conditions, several key variations were tested to account for potential challenges during cell product administration. The DfA hold time post-aliquot from master dose, agitation, in-device hold time, and injection pressure were varied, and key aspects of the dose were the evaluated, including cell viability, viable cell count, aggregate biomass, and extended culture viable cell counts.

More specifically, an extended hold time of greater than 28 hours between 1-8° C. was tested, which exceeds the maximum allowable hold time for the DfA. An extended agitation of 60-75 minutes was also evaluated to account for extended transportation time of the DfA. Additionally, an extended in-device hold time was tested for the DfA held in the device in a vertical orientation for between 15-17 minutes. Finally, the injections were executed at the defined maximum injection pressure setting of 10 psi.

Briefly, a master dose was created at Time 0 (T₀) for two clinical dose targets, 1×10⁶ and 4×10⁶ viable cells, for four replicates. The master doses were assessed for viable cells, total cells, percentage viability, and percentage aggregate biomass at T₀. The master doses were then split across twenty-two samples at 50 μL each, all of which were held at 1-8° C. for 20-21 hours post T₀. Eleven of the samples were then aspirated into the syringe and cannula, held for 15-17 minutes in the vertical orientation, and injected into tubes or cell culture plates for analysis. The remaining eleven samples were pipetted into tubes or cell culture plates for analysis (no aspiration into syringe/cannula). The samples in tubes were evaluated at 28-30 hours after aliquoting for percentage biomass, cell viability, and number of viable cells. The samples in cell culture plates were assessed either the same day as plating or two days after plating. The two-day extended culture viable cell counts were compared to the zero-day controls as a measure of potential downstream impact of the simulated worst-case scenario conditions on cell health.

### Percentage Aggregate Biomass

For percentage aggregate biomass, the master dose and the administered dose were compared. The results are shown in Table 23.

TABLE 23
Percentage Aggregate Biomass of Master
Doses and Administered Doses

| Clinical | | | | | | |
|---|---|---|---|---|---|---|
| Dose | | | Administered Dose | | Administered Dose | |
| Target | Master Dose | | (no aspiration) | | (with aspiration) | |
| (viable | | Standard | | Standard | | Standard |
| cells) | Mean | Deviation | Mean | Deviation | Mean | Deviation |
| 1 × 10⁶ | 87.4% | 3.7% | 87.0% | 3.5% | 84.1% | 2.3% |
| 4 × 10⁶ | 88.5% | 3.5% | 87.4% | 2.8% | 83.9% | 3.3% |

The mean percentage aggregate biomass for the administered doses (both aspirated and non-aspirated) were similar to that of the master doses. This result demonstrates that the cells are suitably aggregated after extended hold, agitation, and passage through the device under simulated worst-case clinical conditions.

### Percentage Viability

For percentage viabilities, the master dose and the administered dose were compared. The results are shown in Table 24.

TABLE 24
Viability and Change in Viability from
Master Dose to Administered Dose

| Clinical | | | | | | |
|---|---|---|---|---|---|---|
| Dose | | | Administered Dose | | Administered Dose | |
| Target | Master Dose | | (no aspiration) | | (with aspiration) | |
| (viable | | Standard | | Standard | | Standard |
| cells) | Mean | Deviation | Mean | Deviation | Mean | Deviation |
| 1 × 10⁶ | 57.6% | 6.6% | 51.0% | 4.9% | 55.2% | 4.4% |
| 4 × 10⁶ | 57.5% | 7.3% | 52.4% | 5.4% | 48.8% | 4.4% |

When compared to the master dose, there was little-to-no change in the mean viability of the administered dose under worst-case conditions for the clinical dose target of 1×10⁶ viable cells, with the aspirated dose demonstrating a 2.4% relative decrease in viability and the non-aspirated dose displaying a 6.6% relative decrease in viability. A small reduction in viability for the clinical dose target of 4×10⁶ viable cells was observed for both the aspirated dose as well as the non-aspirated dose (−8.7% and −5.1% respectively, relative to the master dose viability).

### Extended Culture Viable Cell Counts

The samples in cell culture plates were assessed either the same day as plating or two days after plating for each set of administered samples (aspirated and non-aspirated) to test for longer-term impacts on cell health. The results are shown in Table 25.

TABLE 25
Viable Cell Number Recovered from Culture Plates

| Clinical | | 0 day culture | | 2 day culture | |
|---|---|---|---|---|---|
| Dose | | | Standard | | Standard |
| Target | | Mean | Deviation | Mean | Deviation |
| (viable | | (viable | (viable | (viable | (viable |
| cells) | Treatment | cells) | cells) | cells) | cells) |
| 1 × 10⁶ | no | 1.16 × 10⁶ | 1.2 × 10⁵ | 1.04 × 10⁶ | 8.3 × 10⁵ |
| 4 × 10⁶ | aspiration | 3.82 × 10⁶ | 3.8 × 10⁵ | 3.95 × 10⁶ | 7.2 × 10⁵ |
| 1 × 10⁶ | with | 9.59 × 10⁵ | 1.59 × 10⁵ | 9.65 × 10⁵ | 1.10 × 10⁵ |
| 4 × 10⁶ | aspiration | 3.06 × 10⁶ | 1.22 × 10⁶ | 3.46 × 10⁶ | 7.3 × 10⁵ |

For aspirated or non-aspirated administered doses, when compared to the number of viable cells recovered from 0 day culture controls, the number of viable cells recovered from the plates after 2 days of culture were similar. The results indicate that the integrity of the cell product comprising cell aggregates was maintained in both the aspirated and the non-aspirated conditions. This demonstrates continued cell survival after two days of culture after administration with the delivery system under simulated worst-case conditions for both the low and high clinical dose targets.

### Non-Culture Viable Cell Counts

For those samples in tubes that had been passed through the delivery system (e.g., aspirated), the number of viable cells was assessed 28-30 hours after aliquoting. The results are shown in Table 26.

TABLE 26
Viable Cell Counts

| | |
|---|---|
| | Administered Dose |
| Clinical | (with aspiration) |

| Dose Target | Mean | Standard Deviation |
|---|---|---|
| (viable cells) | (viable cells) | (viable cells) |
| 1 × 10⁶ | 1.06 × 10⁶ | 1.9 × 10⁵ |
| 4 × 10⁶ | 3.38 × 10⁶ | 1.23 × 10⁶ |

The results indicated that injection with the delivery system under simulated worst case clinical conditions resulted in suitable mean counts of 1.06×10⁶ viable cells and 3.38×10⁶ viable cells for the 1×10⁶ and 4×10⁶ clinical dose targets, respectively.

Overall, the combination of results demonstrate compatibility between the cell product comprising cell aggregates and an exemplary delivery system under simulated worst-case conditions. The simulated worst-case conditions resulted in viable cell numbers of 1.06×10⁶±1.9×10⁵ and 3.38×10⁶±1.23×10⁶ for the respective dose targets of 1×10⁶ and 4×10⁶ viable cells. Additionally, the cells delivered for both target clinical doses remained aggregated, exhibited at most a small mean drop in viability from the master dose, and maintained suitable viable cell counts after two days of culture. In summary, the results of this study demonstrate that the delivery system is capable of delivering low and high clinical target doses with minimal impact to cell viability and aggregate integrity under simulated worst-case scenario conditions.

Specific embodiments provided herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

In cases where numerical values are indicated herein, the skilled person will understand that the technical effect of the feature in question is ensured within an interval of accuracy, which typically encompasses a deviation of the numerical value given of ±10% or of ±5%. At the very least, and not as an attempt to limit the application of the doctrine of equivalents to the scope of the claims, each numerical parameter should at least be construed considering the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained.

The terms “a,” “an,” “the” and similar referents used in the context of the description herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the specification and does not pose a limitation on the scope of the claims. No language in the specification should be construed as indicating any non-claimed element essential to the practice of the description.

Unless specifically stated or obvious from context, as used herein, the term “or” is understood to be inclusive and covers both “or” and “and.”

Groupings of alternative elements or embodiments provided herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known for carrying out methods provided herein. Of course, variations on these described embodiments will become apparent upon reading the foregoing description. One can be expected to employ such variations as appropriate and can be practiced other than as specifically described herein. Accordingly, this description includes all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

It is to be understood that the embodiments provided herein are illustrative of the principles of the description herein. Other modifications that can be employed are within the scope of the description. Thus, by way of example, but not of limitation, alternative configurations can be utilized in accordance with the teachings herein. Accordingly, the presented information is not limited to that precisely as shown and described.

While the present description has been described and illustrated herein by references to various specific materials, procedures, and examples, it is understood that the description is not restricted to the particular combinations of materials and procedures selected for that purpose. Numerous variations of such details can be implied as will be appreciated. It is intended that the specification and examples be considered as exemplary only, with the true scope and spirit of the specification being indicated by the following claims. All references, patents, and patent applications referred to in this application are herein incorporated by reference in their entirety.

Unless defined otherwise, all technical and scientific terms used herein have the same meaning as commonly understood. Although other probes, compositions, methods, and kits similar, or equivalent, to those described herein can be used in the practice described herein, the materials and methods are described herein. It is to be understood that the terminology used herein is for the purpose of describing embodiments only and is not intended to be limiting.

Unless specifically stated or obvious from context, as used herein, the term “about” is understood as within a range of normal tolerance, for example within two standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

Any aspect or embodiment described herein can be combined with any other aspect or embodiment as described herein.

1.-92. (canceled)

93. A composition comprising human photoreceptor precursor cells (PRPs), wherein the composition comprises: NR2E3+ cells and between at least 20% to 60% PPP4R4+ cells, and less than or equal to 0.5 copies of VIM per copy of SDHA, and wherein the PRPs are derived from induced pluripotent stem cells (iPSCs).

94. The composition according to claim 93, wherein the human PRPs comprise:
at least 90% AIPL1+ cells;
at least 90% RCVRN+ cells;
less than or equal to 7% PAX6+ cells;
less than or equal to 7% CHX10+ cells;
less than or equal to 0.5% TYRP1+ cells; and
less than or equal to 0.5% Ki67+ cells.

95. The composition according to claim 94, wherein the human PRPs have a percentage viability of at least 50%.

96. (canceled)

97. The composition according to claim 93, wherein the composition comprises cell aggregates having a median diameter between 30 μm to 65 μm.

98. The composition according to claim 93, wherein the human PRPs are stable at 1-8° C. for about 28 hours.

99. The composition according to claim 98, wherein stability is assessed by one or more of percentage viability, viable cell concentration, and/or percentage aggregate biomass.

100. A composition comprising PRPs, wherein the composition comprises cell aggregates, wherein the cell aggregates have a median diameter of about 30 μm to about 65 μm.

101. The composition according to claim 100, wherein the PRPs are in a single cell form, an aggregate cell form, or both.

102. The composition according to claim 101, wherein the PRPs comprise: NR2E3+ cells and between at least 20% to 60% PPP4R4+ cells, and less than or equal to about 0.5 copies of VIM per copy of SDHA.

103. The composition according to claim 102, wherein the PRPs further comprise:
at least about 90% AIPL1+ cells;
at least about 90% RCVRN+ cells;
less than or equal to about 7% PAX6+ cells;
less than or equal to about 7% CHX10+ cells;
less than or equal to about 0.5% TYRP1+ cells; and
less than or equal to about 0.5% Ki67+ cells.

104. The composition according to claim 103, wherein the PRPs have a percentage viability of at least 50%,

105. The composition according to claim 104, wherein the PRPs are derived from human pluripotent stem cells (PSCs) or human induced pluripotent stem cells (iPSCs).

106. The composition according to claim 105, wherein the PRPs are stable at 1-8° C. for about 28 hours.

107. The composition according to claim 106, wherein the PRPs are suitable for grafting to a subretinal space of a subject's eye.

108. A composition comprising human PRPs and balanced salt solution buffer supplemented with 0.2% human serum albumin (HSA).

109. The composition according to claim 108, wherein the balanced salt solution buffer comprises about 0.10 to 1.50 mg/ml of magnesium chloride, about 0.10 mg/ml to about 1.5 mg/ml of calcium chloride, about 0.10 mg/ml to about 1.5 mg/ml of potassium chloride, about 0.10 mg/mL to about 3.0 mg/ml of sodium citrate, about 0.10 mg/mL to about 6.0 mg/ml of sodium acetate, and about 0.10 mg/mL to about 10.0 mg/mL of sodium chloride.

110. The composition according to claim 109, wherein the balanced salt solution buffer comprises 0.3 mg/ml of magnesium chloride, 0.48 mg/ml of calcium chloride, 0.75 mg/ml of potassium chloride, 1.7 mg/ml of sodium citrate, 3.9 mg/ml of sodium acetate, and 6.4 mg/ml of sodium chloride.

111. The composition according to claim 110, wherein the human PRPs are stable at 1-8° C. for about 28 hours.

112. The composition according to claim 111, wherein stability is assessed by one or more of percentage viability, viable cell concentration, and/or percentage aggregate biomass.

113. The composition according to claim 110, wherein the human PRPs comprise NR2E3+ cells and between at least 20% to 60% PPP4R4+ cells, and less than or equal to about 0.5 copies of VIM per copy of SDHA.

114. The composition according to claim 113, wherein the human PRPs further comprise:
at least about 90% AIPL1+ cells;
at least about 90% RCVRN+ cells;
less than or equal to about 7% PAX6+ cells;
less than or equal to about 7% CHX10+ cells;
less than or equal to about 0.5% TYRP1+ cells; and
less than or equal to about 0.5% Ki67+ cells.

115. The composition according to claim 114, wherein the composition comprises about 1×106, about 2×10″, about 3×106, or about 4×106 human PRPs.

116. The composition according to claim 114, wherein the composition comprises:
a) about 2.72×107 total cells/mL to about 4.08×107 total cells/mL;
b) about 5.04×107 total cells/mL to about 7.56×107 total cells/ml;
c) about 7.12×107 total cells/mL to about 1.07×10⁸ total cells/mL; or
d) about 9.44×107 total cells/mL to about 1.42×108 total cells/mL.

117. The composition according to claim 116, wherein the composition has a percentage viability of at least about 50%.

118. The composition according to claim 117, wherein the human PRPs are derived from human pluripotent stem cells (PSCs) or human induced pluripotent stem cells (iPSCs).

119. The composition according to claim 118, wherein the composition further comprises cell aggregates, wherein a median diameter of a cell aggregate is about 30 μm to about 65 um.

120. A composition comprising human PRPs and a cryopreservant solution.

121. The composition of claim 120, wherein the cryopreservant solution comprises adenosine, dextran-40, lactobionic acid, HEPES, sodium hydroxide, potassium chloride, potassium bicarbonate, potassium phosphate, dextrose, sucrose, mannitol, calcium chloride, magnesium chloride, potassium hydroxide, L-glutathione, DMSO, and water.

122. The composition according to claim 121, wherein the human PRPs comprise NR2E3+ cells and between at least 20% to 60% PPP4R4+ cells, and less than or equal to about 0.5 copies of VIM per copy of SDHA, wherein the human PRPs further comprise:
at least about 90% AIPL1+ cells;
at least about 90% RCVRN+ cells;
less than or equal to about 7% PAX6+ cells;
less than or equal to about 7% CHX10+ cells;
less than or equal to about 0.5% TYRP1+ cells; and
less than or equal to about 0.5% Ki67+ cells, and
wherein the composition has a percentage viability of at least about 50%.
